(19) United States
(12) Patent Application Publication
(10) Pub. No.: US 2010/0267049 A1
(43) Pub. Date: Oct. 21, 2010

(54) DIAGNOSTIC DEVICES AND RELATED METHODS

(76) Inventors: William J. RUTTER, San Francisco, CA (US); George Harold Sierra, Shekou (CN); Hongjian Liu, Cupertino, CA (US); Jimmy Z. Zhang, San Francisco, CA (US); Zhihai Ye, San Ramon, CA (US); Alexandre Izmailov, Toronto (CA); Brian David Warner, Martinez, CA (US)

Correspondence Address: MORRISON & FOERSTER LLP, 755 PAGE MILL RD, PALO ALTO, CA, 94304-1018, US

(21) Appl. No.: 12/760,518

(22) Filed: Apr. 14, 2010

Related U.S. Application Data
(60) Provisional application No. 61/169,700, filed on Apr. 15, 2009.
(60) Provisional application No. 61/169,660, filed on Apr. 15, 2009.

(51) Int. Cl. G01N 33/53; C12M 1/34

(52) U.S. Cl. 435/7.1; 435/287.2

(57) ABSTRACT

Devices, systems, and methods for detecting the presence of one or more analytes in a sample are described. In some variations, a test strip may be used to detect and/or analyze one or more analytes in a sample. In certain variations, a test strip configured to receive a sample for detection of an analyte therein may comprise a substrate and a coating on a portion of the substrate, the coating comprising a combination of a first analyte capture agent configured to bind to a first analyte and a second analyte capture agent configured to bind to a second analyte that is different from the first analyte.

### CROSS-REFERENCE TO RELATED APPLICATIONS

This application claims the benefit of U.S. Provisional Application No. 61/169,700, filed on Apr. 15, 2009, and of U.S. Provisional Application No. 61/169,660, filed on Apr. 15, 2009, the disclosures of both of which are incorporated herein by reference in their entirety. Additionally this application is related to U.S. patent application Ser. No. 12/760,320, filed on Apr. 15, 2010, the disclosure of which is incorporated herein by reference in its entirety.

### FIELD

The devices, systems, and methods described herein relate generally to testing for the presence of one or more analytes in a sample. More specifically, the devices, systems, and methods described herein use a combination of at least two different analyte capture agents (at least one of which may be a control analyte capture agent) in the same location on a substrate to test for the presence of one or more analytes in a fluid sample.

### BACKGROUND

Quantitative analysis of cells and analytes in fluid samples, particularly bodily fluid samples, often provides critical diagnostic and treatment information for physicians and patients. One approach to measuring analytes involves assays that take advantage of the high specificity of antigen-antibody reactions. More specifically, an antigen or antibody may be detected in a sample (and, in some cases, may be quantitatively measured) based on binding between the antigen and an antibody on the assay, or vice versa. For example, in a solid-phase immunoassay, a target analyte binding agent (either an antigen or an antibody, depending on the target analyte) may be applied to a substrate. Thereafter, a fluid sample may be applied to the substrate, and the target analyte binding agent may bind to some or all of any target analyte that may be present in the fluid sample. When the target analyte is an antigen, the target analyte binding agent may, for example, be the corresponding antibody, and when the target analyte is an antibody, the target analyte binding agent may, for example, be the corresponding antigen. The extent of binding between the target analyte and the target analyte binding agent may be evaluated to provide a quantitative value for the amount of the target analyte present in the fluid sample. While such assays may be used to evaluate human subjects, they may also find use in various other applications, such as veterinary, food testing, or agricultural applications.

Some assays involve the use of test strips, in which a fluid sample is applied to one location of the test strip, and then travels across a portion of the test strip (e.g., via capillary action) to interact with one or more reagents on the test strip.

For example, a test strip may include a first band comprising a control analyte and a target analyte binding agent, a second separate detection band comprising a target analyte capture agent that binds to the target analyte, and a third separate detection band comprising a control analyte capture agent that binds to the control analyte. During use, a fluid sample may be applied to the test strip, and may travel across at least a portion of the test strip (e.g., via capillary action). When the fluid sample contacts the first band, target analyte in the fluid sample may bind to the target analyte binding agent to form a target analyte complex. When the fluid sample contacts the second band, the target analyte may bind to the target analyte capture agent such that the target analyte complex is immobilized in the second band. Similarly, when the fluid sample contacts the third band, the control analyte may bind to the control analyte capture agent such that the control analyte is immobilized in the third band. The captured target analyte complex and control analyte may then be detected and evaluated to determine the concentration of the target analyte. In some variations, the target analyte binding agent may be conjugated to a first detectable marker and the control analyte may be conjugated to a second detectable marker. The markers may be detected after the target analyte has bound to the target analyte capture agent and the control analyte has bound to the control analyte capture agent, and both analytes have thereby been immobilized in their respective detection bands. The detection may be used to provide a quantitative value for the concentration of target analyte in the fluid sample (normalized by the control).

While such methods and test strips may provide for the detection of analytes in a fluid sample, in some cases, the measured concentration of these analytes may not be highly accurate. For example, the detection bands may be formed of coatings exhibiting variability relative to each other (e.g., as a result of being coated at different times and/or in different locations on the test strip). Such variability may in turn affect the resulting measurement of the concentration of the target analyte or analytes in the fluid sample. In view of the ongoing need to accurately test for certain analytes in, for example, a blood sample, it would be desirable to provide additional assays and related devices and methods for accomplishing such testing with high accuracy.

A variety of diagnostic assays and related devices have been developed for point-of-care (POC) testing. Such diagnostic assays and related devices are generally intended for use in the vicinity of the site of patient care (e.g., at a patient's bedside) or in a de-centralized location other than a reference laboratory. Point-of-care diagnostic assays are intended to provide quick results to the patient in a convenient manner and/or to provide proximity testing when laboratory testing (e.g., at a centralized facility) is not feasible, suitable, or otherwise desirable. Generally, POC devices may be portable or otherwise transportable. In some cases, they may even be handheld. In view of the convenience of POC diagnostic assays and related devices, as well as the timeliness of their results, it would be desirable to provide additional POC assays and diagnostic devices. It would also be desirable to provide POC systems that exhibit high sensitivity, precision, accuracy, and reliability of measurement. Moreover, it would be desirable to provide POC systems that are configured for connectivity with local and/or remote systems.

### SUMMARY

Described here are devices, systems, and methods for evaluating the presence of one or more analytes in a fluid sample, such as a blood sample. Generally, the devices, systems, and methods may test for the presence of at least one analyte in a sample (e.g., a fluid sample) using at least two analyte capture agents (e.g., a target analyte capture agent and a control analyte capture agent) that are combined (e.g., mixed) and/or applied to the same location of a testing medium, such as a test strip. In some variations, devices, systems, and methods described here may be used in POC testing. The devices and systems may be portable and even handheld, and in some cases may be battery-operated. In certain variations, the devices, systems, and/or methods described here may be CLIA-waived (where “CLIA” refers to Clinical Laboratory Improvement Amendments). Systems described here may, for example, be capable of exhibiting high sensitivity and specificity and broad dynamic range. As an example, some variations of systems described here may be capable of reaching an analytical sensitivity of at least 3 pg/mL with a coefficient of variation (CV) of less than 5%. Certain variations of systems described here may be capable of detecting <0.003 ng/mL of cTnI, with a dynamic range spanning 3 logs.

Some variations of devices, system, and/or methods described here may provide relatively quick turnaround time (e.g., providing a benefit in the emergency room). For example, results in some cases may be available in about five minutes.

In some cases, a test strip (e.g., a lateral flow test strip) comprising a substrate and a coating (e.g., in the form of a band) on a portion of the substrate may be used. The coating may include the combination of different analyte capture agents. In certain variations, at least one of the analyte capture agents may be used to detect a target analyte in a fluid sample, while at least one of the other analyte capture agents may be used as a control (e.g., may be used to detect the presence of a control analyte). In such cases, the control may be used to normalize the detection of the target analyte, so that a quantitative value for the concentration of the target analyte in the fluid sample may be established. Certain variations of the devices, systems, and methods described here may employ dual laser-induced fluorescence for measuring target analyte concentration (e.g., with a high signal-to-noise ratio and/or a relatively low coefficient of variation).

Devices, systems, and methods described here may provide for highly reliable, reproducible, and sensitive analyte concentration measurements. For example, some variations of devices, systems, and/or methods described here may be capable of measuring an analyte to an analytical sensitivity of 3 pg/mL or less. In certain variations, the sensitivity of a device or system described here may be 0.003 ng/mL cTnI, 0.2 pg/mL NT-proBNP. Certain variations of devices, systems, and/or methods described here may be capable of measuring multiple (e.g., 10-20) analytes on the same test medium (e.g., a test strip), with a coefficient of variation (CV) 6% or less (e.g., 5.4% at 0.04 ng/mL cTnI), or 5% or less, and/or a dynamic range of 3-5 logs or broader (e.g., >5 logs for NT-proBNP). The time to result (from the addition of the sample) may be within five to ten minutes or less.

In some variations, the devices and/or systems described here may be configured for connection to the Internet or to an intranet (such as HIS—Hospital Information System, or LIS—Laboratory Information System), to a database in a different location, and/or to a remote location. As used herein, a remote location to which the devices and/or systems described herein are connected is a location that is different from the locations of the subject (e.g., patient) and the devices and/or systems during testing (the locations of the subject and the devices and/or systems generally being identical or in close proximity to each other). As an example, a remote location may refer to a different room from the room in which the subject, device and/or system are located, and/or to a location in which the subject, device and/or system cannot be seen. In certain variations, the devices and/or systems described here may be configured for connection to another computer, a server, the Internet and/or an intranet (e.g., via Bluetooth®, Ethernet, LAN, such as wireless LAN, any wireless protocols, or other connection means). Moreover, some variations of devices, systems, and/or methods described here may employ remote monitoring, advising, and/or control (e.g., via phone, Internet, or the like).

The devices, systems, and methods described here may be useful in a number of different applications. For example, they may be used to assay for human diseases, such as infectious diseases (e.g., hepatitis B), or any other human diseases involving recognizable epitopes (e.g. cancer, autoimmune diseases, cardiovascular conditions, hormone testing, and pathology). Some variations of devices, systems, and/or methods described here may be used to test for substance abuse. The assays may also be used in veterinary, food testing, agricultural, or fine chemical applications, and the like. In certain variations, the devices, systems, and/or methods described here may be used in chemistry gas testing or nucleic acid testing, for example, oxygen content detection and nucleic acid detection.

In certain variations, a test strip or other testing medium configured to receive a sample for detection of an analyte therein may comprise a substrate and a coating on a portion of the substrate, the coating comprising a combination of a first analyte capture agent configured to bind to a first analyte and a second analyte capture agent configured to bind to a second analyte (e.g., a control analyte) that is different from the first analyte. Analyte capture agents for use with the devices, systems, and methods described herein may be selected from the group consisting of antibodies, engineered proteins, peptides, haptens, lysates containing heterogeneous mixtures of antigens having analyte binding sites, ligands, and receptors.

In some variations, the coating may comprise a mixture of the first and second analyte capture agents. In certain variations, the first and second analyte capture agents may be labeled with detectable markers, such as fluorophores. For example, the first analyte capture agent may be labeled with a first fluorophore, and/or the second analyte capture agent may be labeled with a second fluorophore (e.g., that is different from the first fluorophore). The substrate may comprise nitrocellulose. The coating may form a first band on the substrate. The test strip may further comprise a second band configured for addition of the sample thereto. One or more of the bands may at least partially overlap. The first band may be at least about 2 millimeters (mm) and/or at most about 5 mm from the second band.

In the test strips or other testing media described here, capture and/or binding agents may be directly and/or indirectly labeled (e.g., with a fluorophore). In some cases, antibodies that are directly labeled may be used. In certain cases, streptavidin may be used to label capture and/or binding agents (e.g., with a fluorophore).

Directly labeled agents and/or indirectly labeled agents may be used in the test strips or other testing media described here. In some cases, direct-labeled antibodies may be used. In certain cases, streptavidin may be used.

In certain variations, a method for detecting at least one analyte in a sample may comprise applying the sample to a portion of a test strip (or other testing medium) comprising a coating comprising a first analyte capture agent configured to bind to a first analyte and a second analyte capture agent configured to bind to a second analyte (e.g., a control analyte) that is different from the first analyte, and applying light to the test strip, where the application of light to the test strip provides an indication of whether the first analyte is present in the sample. In some variations, the sample may be applied directly to the portion of the test strip comprising the coating comprising the first and second analyte capture agents. In other variations, the sample may be indirectly applied to the portion of the test strip (e.g., by being applied to a sample pad that is in contact with the portion of the test strip).

The method may further comprise measuring the concentration of the first analyte in the sample. Applying light to the test strip may comprise applying light from first and second light sources to the test strip. At least one of the first and second light sources may comprise a laser. For example, the first light source may comprise a first laser and the second light source may comprise a second laser that is different from the first laser.

The test strip may further comprise an analyte binding agent and a control analyte (e.g., in a different band from the first and second analyte capture agents). The analyte binding agent may be labeled with a first fluorophore that fluoresces upon exposure to light from the first light source. Alternatively or additionally, the control analyte may be labeled with a second fluorophore that fluoresces upon exposure to light from the second light source. Measuring the concentration of the first analyte in the sample may comprise comparing the intensity of the fluorescence of the first fluorophore to the intensity of the fluorescence of the second fluorophore. In variations in which the second analyte comprises the control analyte, measuring the concentration of the first analyte in the sample may comprise using a processor, memory resources, and software to evaluate the amount of the first analyte capture agent that is bound to the first analyte relative to the amount of the second analyte capture agent that is bound to the second analyte. The processor, memory resources, and software may analyze the test strip in a period of less than twenty minutes (e.g., less than ten minutes) after the sample has been applied to the portion of the test strip.

The sample may comprise a fluid sample such as blood. In some variations, the method may further comprise passing the sample through a filter before applying the sample to the portion of the test strip. In certain variations, a liquid sample may be prepared for testing by dissolving one or more solutes in a solvent to form a solution.

In some variations, a method of making a test strip or other testing medium configured to receive a sample for detection of an analyte therein may comprise combining a first analyte capture agent with a second analyte capture agent to form a coating material, where the first analyte capture agent is configured to bind to a first analyte and the second analyte capture agent is configured to bind to a second analyte (e.g., a control analyte) that is different from the first analyte. In some variations, the method may further comprise applying the coating material to a portion of a substrate to form a coating on the substrate.

In certain variations, a point-of-care system for detecting an analyte in a sample may comprise an apparatus comprising a first laser and a second laser that is different from the first laser. The system may further comprise a test strip (or another suitable testing medium). In some variations, the system may comprise a housing comprising a receptacle, and the test strip may be configured to fit within the receptacle. In some such variations, the first laser may be configured to apply a first beam to the test strip when the test strip is positioned in the receptacle, and the second laser may be configured to apply a second beam to the test strip (e.g., to the same location on the test strip where the first beam is or was applied) when the test strip is positioned in the receptacle.

The apparatus may further comprise at least one mirror configured to direct application of at least one of the first and second beams to the test strip. In some variations, the apparatus may further comprise an objective lens configured to receive light emitted from the test strip. In certain variations, the apparatus may further comprise a first detector configured to detect light emitted from the test strip and received through the objective lens.

The test strip may comprise a substrate and a coating on a portion of the substrate, the coating comprising a first analyte capture agent configured to bind to a first analyte and a second analyte capture agent configured to bind to a second analyte that is different from the first analyte. The test strip may also comprise an analyte binding agent and a control analyte. In some variations, the analyte binding agent and the control analyte may be labeled with detectable markers. For example, the analyte binding agent may be labeled with a first fluorophore and the control analyte may be labeled with a second fluorophore. The first laser may emit light at a wavelength within the excitation spectrum of the first fluorophore, and/or the second laser may emit light at a wavelength within the excitation spectrum of the second fluorophore.

The apparatus may further comprise an objective lens configured to receive light emitted from the location of the receptacle, and may comprise a first detector configured to detect light emitted from the location of the receptacle and received through the objective lens. The first detector may be configured to detect fluorescence from the first fluorophore. The apparatus may further comprise a second detector configured to detect fluorescence from the second fluorophore. In some variations, the apparatus may further comprise a filter (e.g., a dichroic filter) configured to separate fluorescence from the first fluorophore from fluorescence from the second fluorophore. The apparatus may further comprise a photodiode.

The first and/or second lasers may emit light at a wavelength of about 300 nm to about 800 nm. In certain variations, the first laser may emit light at a different wavelength from the second laser. The first laser may comprise a laser emitting in the red region of spectrum. The second laser may comprise an infrared laser. At least one of the first and second lasers may be a fiber-coupled laser.

The apparatus may, for example, be configured to measure the concentration of the first analyte to an analytical sensitivity of <3 pg/mL. In some variations, the apparatus may be configured to measure the concentration of the first analyte to an analytical sensitivity of at least 3 pg/mL with a coefficient of variation of less than 5%.

The system may be configured to detect a plurality of analytes in a sample. For example, the system may be configured to detect from 10 to 20 analytes on the test strip.

In certain variations, a method for detecting at least one analyte in a sample may comprise applying the sample to a test strip (or another testing medium), applying a first beam from a first laser of a point-of-care diagnostic system to the test strip, and applying a second beam from a second laser of the point-of-care diagnostic system to the test strip (e.g., to the same location on the test strip where the first beam is or was applied), where the application of the first and second beams to the test strip provides an indication of whether the analyte or analytes are present in the sample. The first and second beams may be applied to the test strip simultaneously.

In some variations, a method may comprise adding a sample obtained from a subject to a point-of-care diagnostic system configured to obtain data from the sample regarding the presence or absence of one or more analytes therein, and to transmit the data in real time to a remote location where the data may be evaluated and/or incorporated into a medical record of the subject. In certain variations, a method may comprise adding a sample obtained from a subject to a point-of-care diagnostic system, where the point-of-care diagnostic system is configured for operation by an operator in a remote location.

The remote location may be at least about 20 feet (e.g., at least about 50 feet, at least about 100 feet, at least about 500 feet, at least about one mile, at least about 5 miles, at least about 10 miles, at least about 25 miles, at least about 50 miles, etc.) from the point-of-care diagnostic system. The point-of-care diagnostic system may be configured to transmit data obtained from the sample to the remote location in real time. In certain variations, the subject may add the sample to the point-of-care diagnostic system, and/or the sample may be added to the point-of-care diagnostic system in a non-clinical setting. In certain variations, the point-of-care diagnostic system may be configured for operation by an operator without medical training. In some variations, the point-of-care diagnostic system may be configured to transmit the data to the remote location telephonically, via the Internet, and/or via an intranet. In certain variations, the point-of-care diagnostic system may be configured for telephonic operation, operation via the Internet, and/or operation via an intranet.

The point-of-care diagnostic system may comprise a test strip, and adding the sample to the point-of-care diagnostic system may comprise applying the sample to the test strip. In some variations, the test strip may comprise a substrate and a coating on a portion of the substrate, the coating comprising a combination of a first analyte capture agent configured to bind to a first analyte and a second analyte capture agent configured to bind to a second analyte that is different from the first analyte. In certain variations, the data may include the concentration of at least one of the first and second analytes.

The point-of-care diagnostic system may comprise an apparatus comprising a first laser, a second laser, and a housing comprising a receptacle, and a test strip configured to fit within the receptacle. In some variations, adding the sample to the point-of-care diagnostic system may comprise applying the sample to the test strip when the test strip is positioned in the receptacle. In certain variations, the method may further comprise applying a first beam from the first laser to the test strip, and applying a second beam from the second laser the test strip. The first and second beams may be applied to the same location on the test strip in some cases.

The operator may, for example, be a medical professional (e.g., a doctor, a nurse, etc.). In some variations, the point-of-care diagnostic system may be configured to be automatically refilled or replenished.

### BRIEF DESCRIPTION OF THE DRAWINGS

FIG. 1A is a cutaway perspective view of one variation of a point-of-care diagnostic system.

FIG. 1B is a cutaway perspective view of another variation of a point-of-care diagnostic system, and FIG. 1C is a cutaway front view of the system of FIG. 1B.

FIG. 1D is a perspective view of the system of FIG. 1A with a housing.

FIG. 2A is a perspective view of a variation of a cartridge for a point-of-care diagnostic system.

FIG. 2B is a perspective view of another variation of a cartridge for a point-of-care diagnostic system.

FIGS. 3A-3C depict variations of a test strip and a method of using the test strip to detect the presence of one or more analytes in a fluid sample.

FIG. 3D depicts a cross-section of a variation of a test strip.

FIG. 4A is a flowchart representation of a variation of a method for forming a contact band (also known as a conjugate pad) on a test strip.

FIG. 4B is a flowchart representation of a variation of a method for forming a sample detection band on a test strip.

FIG. 4C is a flowchart representation of a variation of a method for making a cartridge for retaining a test strip.

FIG. 4D is a flowchart representation of a variation of a method for assembling a cartridge kit.

FIG. 5A is a perspective view of one variation of an optical module of a point-of-care diagnostic system.

FIG. 5B is a cutaway perspective view of another variation of an optical module of a point-of-care diagnostic system (the view including a cartridge as a frame of reference), and FIG. 5C is a perspective view of the components of the optical module of FIG. 5B, removed from the optical module housing.

FIG. 6 is an illustrative depiction of another variation of an optical module of a point-of-care diagnostic system (including a sample holder as a frame of reference).

FIG. 7A is a perspective view of a variation of an excitation module of an optical module of a point-of-care diagnostic system, and FIG. 7B is a side view of the excitation module of FIG. 7A.

FIG. 7C is an illustrative depiction of another variation of an excitation module of an optical module of a point-of-care system (including a cartridge and an objective lens or detection module as a frame of reference).

FIGS. 7D-7H are illustrative depictions of excitation modules of optical modules of point-of-care systems (including cartridges and objective lenses as frames of reference).

FIGS. 7I-7L depict variations of components of excitation modules.

FIG. 7M illustrates variations of an excitation module and a related method for using the excitation module to test for the presence of one or more analytes on a test strip; FIG. 7N is a perspective view of a variation of a fiber-coupled laser; and FIG. 7O is a side view of the fiber-coupled laser of FIG. 7N.

FIG. 7P is an illustrative depiction of an excitation module of an optical module of a point-of-care system (including a cartridge and an objective lens as a frame of reference).

FIG. 8A is a perspective view of a variation of a detection module of a point-of-care diagnostic system, and FIG. 8B is a side view of the detection module of FIG. 8A.

FIG. 9A is a perspective view of a variation of an objective lens unit of a detection module; FIG. 9B is an exploded view of the objective lens unit of FIG. 9A; and FIGS. 9C-9E are perspective views of a component of the objective lens unit of FIGS. 9A and 9B.

FIG. 10 is an illustrative cross-sectional view of a variation of an objective lens unit of a detection module of a point-of-care diagnostic system (including a sample holder as a frame of reference).

FIG. 11A is a perspective view of an assembly of two detector units of a detection module of a point-of-care diagnostic system; FIG. 11B is an exploded view of one of the detector units of FIG. 11A; and FIG. 11C is a cross-sectional view of the detector unit of FIG. 11B.

FIG. 12 is an illustrative cross-sectional depiction of a variation of a detection module of a point-of-care diagnostic system (including a sample holder as a frame of reference).

FIG. 13 depicts another variation of a detection module of a point-of-care diagnostic system.

FIG. 14A is a top perspective view of a variation of a motorized sample-holding tray of a point-of-care diagnostic system; FIG. 14B is a top view of the tray of FIG. 14A; FIG. 14C is another top perspective view of the tray of FIG. 14A; FIGS. 14D and 14E are perspective and cross-sectional views of a heater bar and circuit board; FIGS. 14F and 14G are bottom perspective views of the tray of FIG. 14A; FIG. 14H is a bottom view of the tray of FIG. 14A; and FIG. 141 is a side view of the tray as shown in FIG. 14F, taken from line 14I-14I.

FIGS. 15A and 15B are perspective views of a variation of a sample holder of a point-of-care diagnostic system, and FIG. 15C is a side view of the sample holder of FIGS. 15A and 15B.

FIGS. 16A-16C are schematic representations of variations of point-of-care diagnostic systems.

FIG. 16D is an illustrative depiction of a variation of an excitation module of a point-of-care diagnostic system.

FIG. 16E is an illustrative depiction of another variation of an excitation module of a point-of-care diagnostic system.

FIG. 17A is a partial cutaway perspective view of one variation of an embedded computing system comprising a hard drive for use with a point-of-care diagnostic system.

FIG. 17B is a block diagram representing a variation of a computer software architecture for use with a point-of-care diagnostic system.

FIG. 17C is a block diagram representing a variation of a computer for use with a point-of-care diagnostic system.

FIG. 18 is a standard curve from an assay described in Example la.

FIG. 19 is a graphical representation depicting the analytical sensitivity of a cTnI assay described in Example 2.

FIG. 20 depicts experimental results from a multiplex assay described in Example 4.

FIG. 21 is an illustration of a test strip configuration described in Example 5.

FIG. 22 is a graphical representation of experimental results of an assay described in Example 5.

FIG. 23 is another graphical representation of experimental results of an assay described in Example 6.

FIG. 24A is a partial cutaway perspective view of one variation of an excitation module of the point-of-care diagnostic system of FIG. 1A, and FIG. 24B is a partial cutaway side view of the excitation module of FIG. 24A.

FIG. 25A is an illustrative depiction of a variation of a detection module of the point-of-care diagnostic system of FIG. 1A.

FIG. 25B a partial cutaway view of an optical lens unit of the detection module of FIG. 25A.

FIGS. 25C and 25D are top and bottom perspective views of a variation of a dichroic filter of the optical lens unit of FIG. 25B.

FIG. 25E is an illustrative depiction of a variation of a light path through the detection module of FIG. 25A.

FIG. 25F is a partial cross-section of a variation of a detector unit of the detection module of FIG. 25A (with a dichroic filter and objective lens as a frame of reference).

FIGS. 26A and 26B depict partial cutaway view of the point-of-care diagnostic system of FIG. 1A without the housing and optical module.

FIG. 26C depicts one variation of a tray housing and movable tray assembly of the point-of-care diagnostic system of FIG. 1A.

FIG. 27A is a partial cutaway view of the movable tray assembly of FIG. 26C.

FIGS. 27B-27D are perspective cutaway views of one tray movement mechanism of the movable tray assembly of FIG. 26C.

FIGS. 27E-27I are top views of the various horizontal and transverse configurations of a variation of a tray.

FIGS. 28A and 28B are partial cutaway views of one position detection mechanism that is used with the tray movement mechanism of FIGS. 27B-27D.

FIGS. 29A-29C are perspective and partial cutaway cross-sectional views of a sample stage mounted on a tray plate of the movable tray assembly of FIG. 26C, with FIGS. 29B and 29C depicting one variation of a fluid sensor and a heating element that are used with the sample stage and tray plate of FIG. 29A.

FIG. 30 is a graphical representation of experimental results of an assay described in Example 7.

FIG. 31 is another graphical representation of experimental results of an assay described in Example 8.

FIG. 32 is an additional graphical representation of experimental results of an assay described in Example 9.

FIG. 33 is another graphical representation of experimental results of an assay described in Example 10.

FIG. 34 is a graphical representation of experimental results of an assay described in Example 11.

FIG. 35 is another graphical representation of experimental results of an assay described in Example 12.

### DETAILED DESCRIPTION

Described here are devices, systems, and related methods for assaying a fluid sample to detect one or more analytes in the fluid sample. In some variations, the concentration of the analyte or analytes in the fluid sample may be measured, as well. Generally, the methods and devices described here may involve test strips having a coated portion including at least two different analyte capture agents. For a given test strip, the analyte capture agents are therefore located at the same site on the test strip. In some cases, at least one of the analyte capture agents may be a control analyte capture agent. In such cases, at least one of the other analyte capture agents may be used to detect the presence of a target analyte, and the concentration of the target analyte may be measured and normalized using the control. Without wishing to be bound by theory, it is believed that locating a target analyte capture agent and a control analyte capture agent in the same place on a test strip may result in less likelihood for error and/or variation in measurement, and may lead to better reproducibility and reliability of results. Additionally, in some cases, the target analyte capture agent and the control analyte capture agent may be mixed at the same time (e.g., in the same tube) and may also be coated onto a substrate at the same time. This may also result in a reduction in the errors and variations that may occur with other methods.

In certain variations, the test strips and other components and/or methods described herein may be used in POC diagnostic systems. When appropriate, they may also be used in other types of systems, such as other types of in vitro diagnostic systems (IVD). Additionally, features of POC diagnostic systems described herein, as well as related methods, may be applied to other types of systems, as appropriate. Moreover, in some variations, systems and methods having one or more features described herein may not use test strips. In certain cases, the systems described here may be relatively inexpensive to manufacture, and thus may be made widely available. Moreover, some variations of the systems, such as the POC systems, may be used to provide quantitative analysis of samples (e.g., fluid samples) in a relatively short period of time (e.g., 60 minutes or less, 30 minutes or less, 20 minutes or less, or ten minutes or less, such as five to ten minutes, from the time of taking the sample).

### System Overview

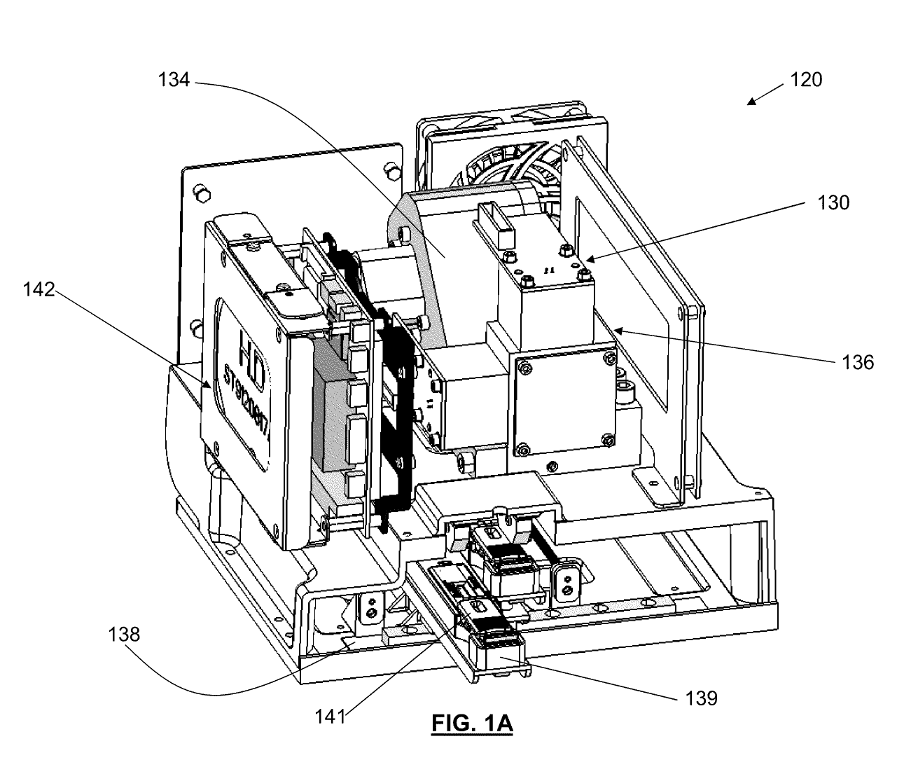

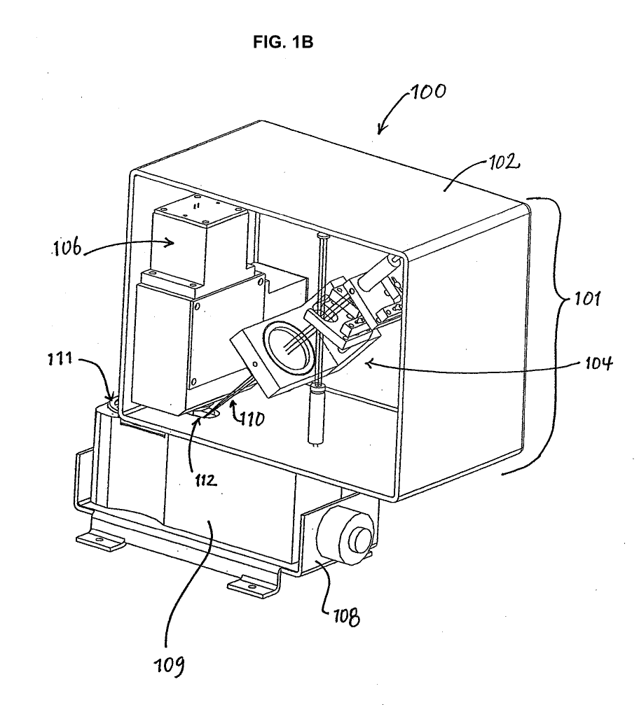

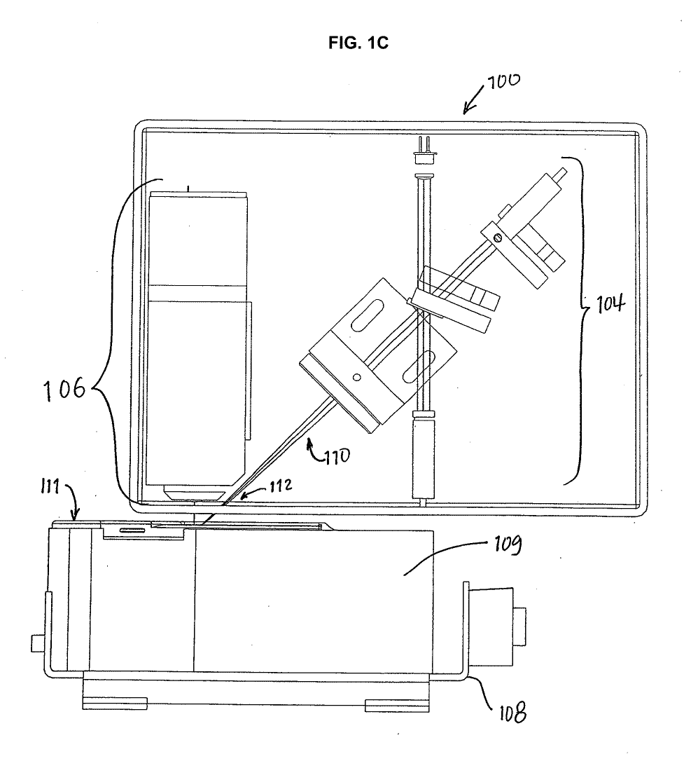

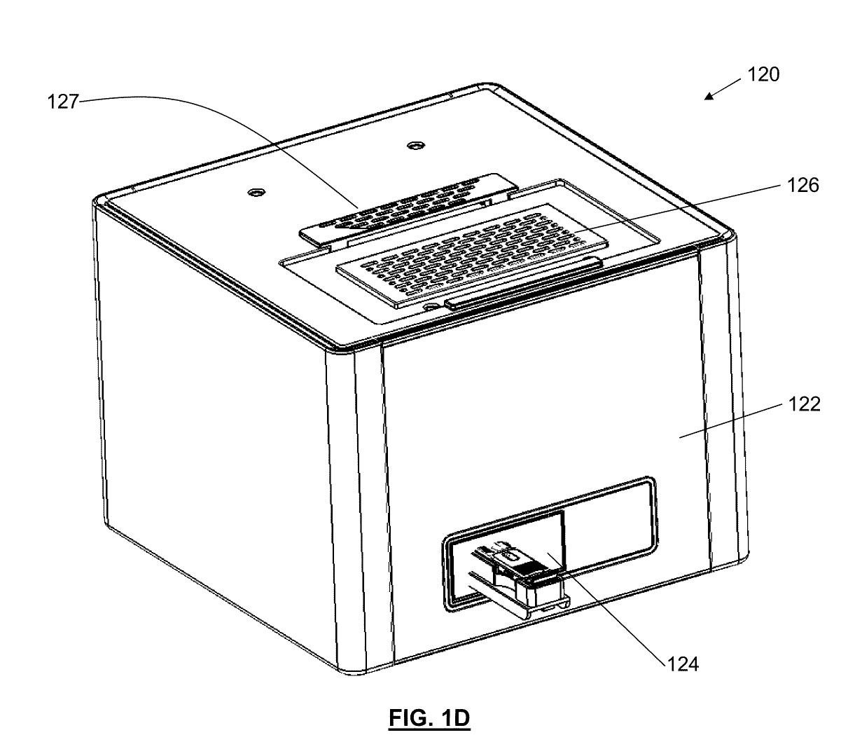

Turning now to the figures, FIG. 1A depicts a partial cutaway perspective view of a variation of a POC diagnostic system (120). System (120) may be used to assay a fluid sample on a test strip retained by a sample cartridge (141), to detect and/or measure the concentration of one or more analytes in the fluid sample.

As shown in FIG. 1A, system (120) comprises an optical module (130) that, in turn, comprises an excitation module (134) and a detection module (136). System (120) also comprises a stage or movable tray (138), which may be used to position sample cartridge (141) with respect to optical module (130). In some cases, sample cartridge (141) may be retained by a first sample stage (139) that may be mounted on movable tray (138). Any suitable number of sample stages may be included in system (120) depending, for example, on the number of sample cartridges to be analyzed and the capacity of movable tray (138).

During use, and as will be described in more detail below, laser beams from excitation module (134) may illuminate a portion of the test strip that is located in sample cartridge (141). The resulting light (e.g., of fluorescence) may then be detected by detection module (136), which may provide an indication to an operator that one or more analytes are present in the sample on the test strip. In some cases, the results may be further analyzed to determine the concentration of at least one of the analytes in the sample. In certain variations, system (120) may comprise an embedded computing device (142) that may perform one or more analyses on the light detected by detection module (136). to provide qualitative and/or quantitative analyte data to the operator.

FIGS. 1B and 1C show cutaway perspective and front views, respectively, of another variation of a POC diagnostic system (100). As shown there, system (100) comprises an optical module (101) comprising a housing (102) containing an excitation module (104) and a detection module (106). System (100) also comprises a stage or motorized tray (108) comprising a sample holder (109). Tray (108) is configured to move beneath housing (102). Sample holder (109) holds a cartridge (111) that contains a test strip (not shown) on which a sample has been applied for testing. During use, laser beams (110) from excitation module (104) pass through an aperture (112) in housing (102), and illuminate a portion of the test strip that is positioned beneath aperture (112). The resulting light is then detected by detection module (106) and analyzed to provide a qualitative and/or quantitative indication to the operator that one or more analytes are present in the sample on the test strip. It should be noted that certain structural components have been omitted from FIGS. 1B and 1C. For example, excitation module (104) further includes components that help to couple certain of its other components to housing (102), but that are not shown in FIGS. 1B and 1C.

Diagnostic systems such as the variations described above may comprise a housing that encloses the optical module and/or a sample cartridge loaded therein. The housing may provide a controlled incubation environment for the sample cartridge while also protecting the sample cartridge from contamination, unintended fluctuations in temperature, and the like. In some variations, a system for a light-based assay may comprise a housing that is configured to regulate the light level in the vicinity of the sample cartridge. For example, the housing may be light-tight, which may help improve the signal-to-noise ratio of the light detected by the detector module, and may also protect the operator from any light (e.g., laser light) that may be emitted from the excitation module.

One example of a housing that may be used to encase a diagnostic system is shown in FIG. 1D. As shown there, the housing (122) comprises an aperture (124) that may be sized and shaped for accommodating a sample cartridge and/or sample tray therethrough. Additionally, housing (122) comprises one or more slits (126) in a portion that is open to the air. Optionally, housing (122) may also comprise apertures or slits as part of an interface (127) between the internal components of the diagnostic system and one or more external components (e.g., display, network devices, keyboard, mouse, etc.). Additionally, certain variations of diagnostic system housings or covers may comprise one or more handles, grooves, straps, and/or other features that may be used to transport the diagnostic system from one location to another.

Systems described here may be relatively easy to operate. In some cases, the systems may be operable by non-technical personnel. It should be understood that features, characteristics, and components of any of the systems, devices, and methods described here may be applied to other systems, devices, and methods described here, as appropriate. The various components of systems (100) and (120) will now be described in further detail below.

### Cartridge

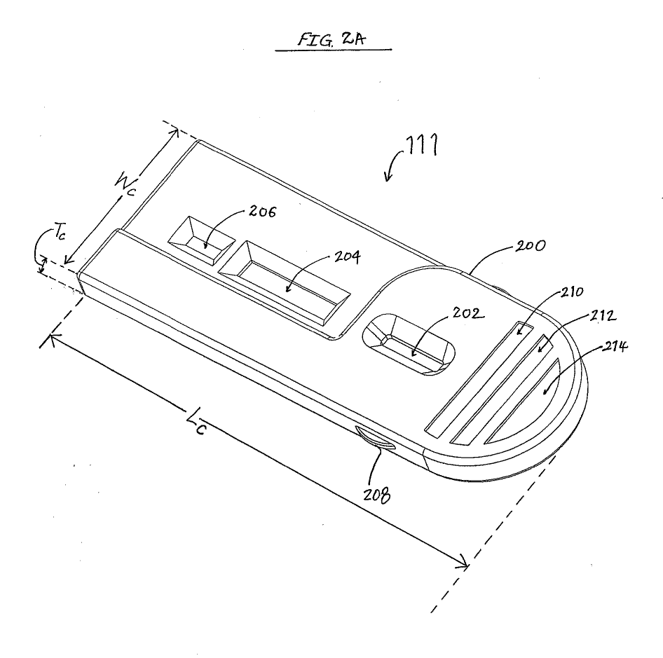

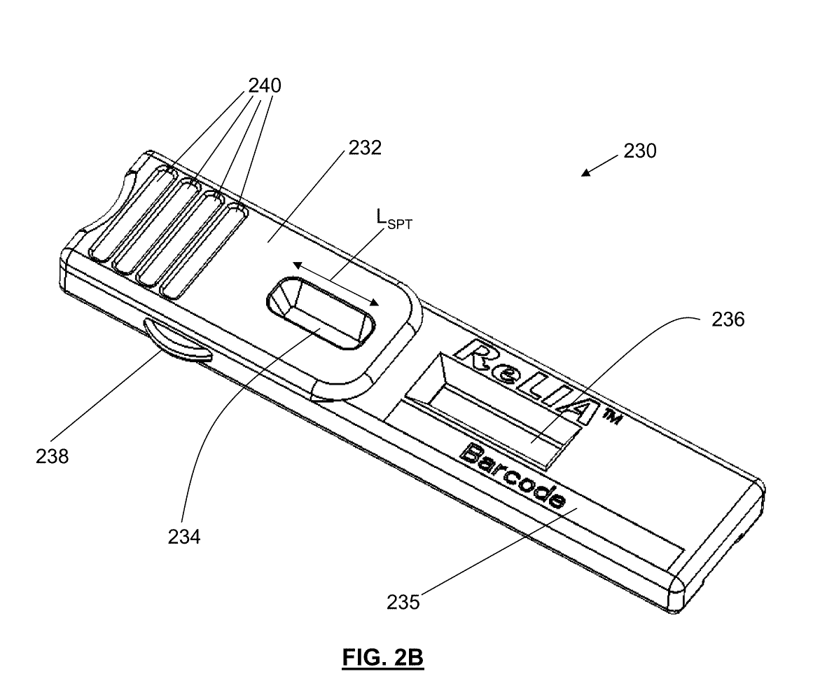

Referring now to FIG. 2A, cartridge (111) comprises a cartridge housing (200) having multiple apertures therein, including a first port (202), a test strip-viewing aperture (204), and an optional second port (206). Cartridge housing (200) may also comprise a variety of handling features, such as grooves (210), (212), and (214), which may allow for a secure hold on the cartridge. A test strip may be enclosed in cartridge housing (200) by any suitable configuration of snap-clasps, hooks, and other types of closure mechanisms. In certain variations, during use, cartridge housing (200) may be opened by releasing the closure mechanism(s), and a test strip (not shown) may be positioned therein. In some variations, a test strip may be permanently sealed in cartridge (111) during manufacturing. Cartridge (111) also comprises a protrusion (208) that may be of any appropriate size or shape to secure the cartridge into the cartridge tray (which will be shown and described in detail below), such that the cartridge may contact the appropriate tray structures precisely and consistently.

The test strip may be positioned within cartridge (111) such that it is disposed beneath first port (202), test strip-viewing aperture (204), and second port (206). Additionally, the test strip may have a wicking portion that may be disposed at or in the proximity of optional aperture (206) in cartridge housing (200). In some variations, the wicking portion may be disposed along the width of the cartridge, perpendicular to the axis defined by apertures (202), (204), and (206).

As shown in FIG. 2A, cartridge housing (200) has a length (L_{C}), a width (W_{C}), and a thickness (T_{C}). In some variations, length (L_{C}) may be from about 60 millimeters (mm) to about 80 mm, width (W_{C}) may be from about 15 mm to about 30 mm, and/or thickness (T_{C}) may be from about 1 mm to about 6 mm. While cartridge housing (200) has a particular configuration as shown, other variations of cartridge housings may have different configurations. As an example, while cartridge housing (200) is configured to hold one test strip, some variations of cartridge housings may be configured to hold multiple test strips, such as two, three, four, or five test strips. In some variations, a sample holder and/or cartridge may be bar-coded (e.g., to store assay specific information). The barcode may, for example, be located on the cartridge housing. A cartridge housing may comprise any appropriate material or materials, such as a polymer or a combination of different polymers.

Another variation of a cartridge (230) is shown in FIG. 2B. Cartridge (230) comprises a cartridge housing (232) having multiple apertures therein, including a port (234) and a test strip-viewing aperture (236). Additionally, cartridge housing (232) comprises a protrusion (238) and indentations/grooves (240). As previously described, protrusion (238) may, for example, be used to ensure correct alignment of cartridge (230) when it is placed in a cartridge tray (described in detail below), and grooves (240) may, for example, provide an operator with a better grip on the cartridge. Port (234) may be used for sample application, while aperture (236) may allow for sample viewing.

While a cartridge having a specific port and aperture has been shown, a cartridge may comprise any number, shape, and/or size of apertures, which may be arranged in a suitable way to accommodate a sample for testing and measurement. Referring back to cartridge (230), port (234) may be sized and shaped to accommodate a fluid sample therethrough. For example, port (234) may have a length (L_{SPT}) from about 5 mm to about 15 mm (e.g., 7.4 mm, or 10 mm). The dimensions of port (234) may be selected to accommodate a specific fluid sample volume. In some variations, port (234) may be dimensioned to accommodate fluid samples having volumes ranging from about 20 microliters (μL) to about 120 μL (e.g., 55 μL to 60 μL, or 100 μL).

Cartridge (230) may also comprise at least one identification feature (235), such as a barcode or a radio frequency identification device (RFID). Identification feature (235) may store information that can be scanned and/or decoded by a diagnostic system during use. For example, a barcode or RFID may contain information such as assay type, lot number, expiration date, patient information, instructions, etc. In some variations, the data encoded in a barcode or RFID tag may include assay data in the form of an assay table, as well as a lot number. An assay table may include, for example, instructions to a computing device on how to analyze the data for a particular assay, as well as information such as calibration curves, standard curves, the number of expected bands on the test strip, incubation time, assay name, analyte type, cut off constant, curve fit parameters and models, etc. The lot number may, for example, indicate the location of the capture analyte bands on the test strip, as well as the number of expected bands.

### Test Strip

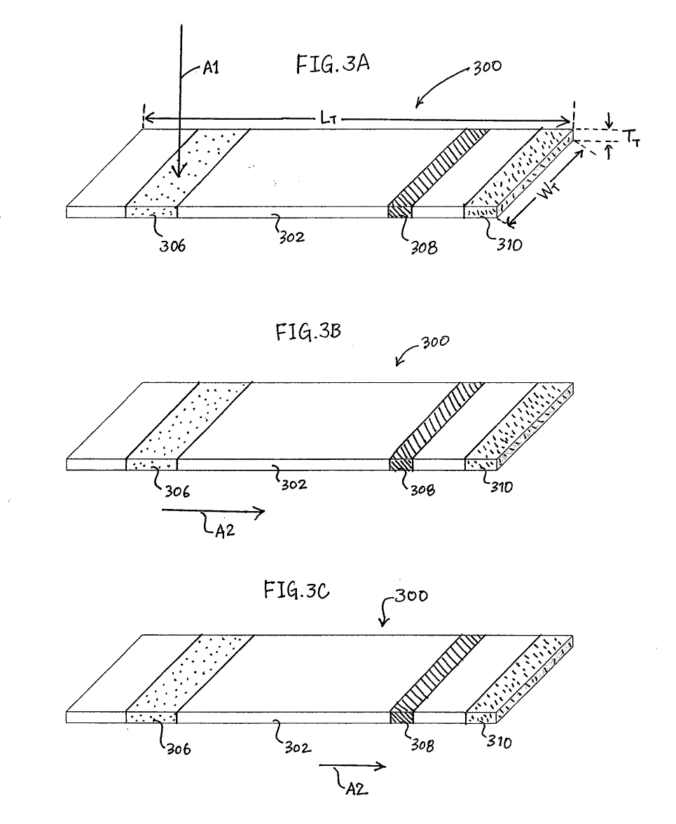

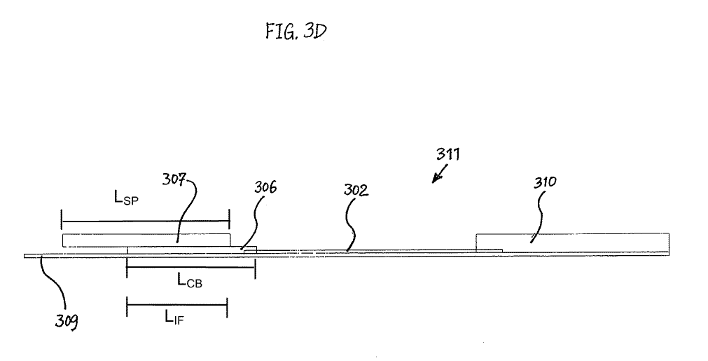

FIGS. 3A-3C depict a variation of a test strip (300) that may be used, for example, in cartridge (111), and a related method for testing a sample for one or more analytes using the test strip.

As shown in FIG. 3A, test strip (300) has a length (L_{T}), a width (W_{T}), and a thickness (T_{T}). In certain variations, length (L_{T}) may be from about 20 mm to about 70 mm, for example, 25 mm. In some variations, length (L_{T}) may be from about 10 mm to about 60 mm, for example, 16 mm. Alternatively or additionally, width (W_{T}) may be from about 2 mm to about 3 mm, for example, 3 mm or 3.4 mm, and/or thickness (T_{T}) may be less than about 2 mm (e.g., less than about 1 mm). While not shown, in certain variations, the thickness of a test strip may vary in different regions of the test strip. As an example, one region of the test strip may have a thickness of about 1 mm to about 2 mm, while another region of the test strip has a thickness of less than about 1 mm.

While test strip (300) is depicted as having a generally rectangular and symmetrical shape, other variations of test strips may have different shapes. For example, instead of being angular, a test strip may be more rounded, and/or may have an asymmetrical shape. The shape of a test strip may depend, for example, on the shape of a cartridge to be used with the test strip. Moreover, in some variations, a test strip may not be used. Rather, a testing medium or substrate having a different configuration (e.g., in the shape of a circle such as a dot, or an oval, or any other appropriate shape) may be employed. For certain assays, test strips with certain sizes or shapes (e.g., test strips with relatively small dimensions) may allow for a relatively fast measurement. It should be understood that features of test strips described here, as well as related methods, may be applied to other substrates or testing media, as appropriate.

Referring again to FIG. 3A, test strip (300) comprises a substrate (302), a contact band (or conjugate pad) (306), a sample detection band (308), and a wicking portion (or absorbent pad) (310). Wicking portion (310) helps to pull fluid through test strip (300) and is in fluid contact with substrate (302). While not shown, in some variations, there may be a sample application band that is separate from contact band (306). While the contact, sample detection, and wicking portions are depicted here as rectangular stripes, in some variations, they may have alternate geometries such as circular dots, ovals, ellipses, hexagons, and the like. During use, a fluid sample may be applied to the sample application band and may subsequently be drawn toward the contact band. While the flow of the fluid sample in this variation may generally be linear and continuous, in some variations the flow of a fluid sample on a test strip may not be linear and/or may not be continuous. For example, in certain variations, the flow may be at 90° or even at 180° (bi-lateral flow). Other types of flow may also occur.

In certain variations, contact band (306) and sample detection band (308) may be separated by a distance of about 3 mm to about 5 mm, and/or sample detection band (308) and wicking portion (310) may be separated by a distance of about 1 mm to about 10 mm. The distance between specific bands and/or portions of a test strip may be selected, for example, based on the distance that the sample must travel in order to be detected, and/or based on the properties of the sample, the control, the analyte binding agents, and/or the test strip substrate. It may be desirable for bands to be separated by a short distance when the test strip is configured to detect multiple analytes. Each band on a test strip may have the same general dimensions (length, width, thickness, and surface area), or at least some of the bands may have different dimensions. In some variations, a band may have a width of about 0.7 mm to about 2 mm.

Some variations of test strips may further comprise a backing strip. A cross-section of a test strip (311) comprising a backing strip (309) is shown in FIG. 3D. The backing strip may, for example, run the length of the test strip, or may only be used on a portion of the test strip. Backing strips may generally be made of any stable, non-porous material or materials that are sufficiently strong to support the materials coupled to them. Since many assays employ water as a diffusion medium, backing strips is preferably are substantially impervious to water. In one variation, a backing strip may be made of a polymer film, such as a polyvinyl chloride (PVC) film. Certain variations of test strips may comprise a protective cover, either as an alternative to, or in addition to, comprising a backing strip. Protective covers may be formed of, for example, one or more water-impermeable materials, and in some variations may be translucent or transparent (e.g., depending on the method of detection that is employed). Exemplary materials for use in a protective cover include optically transmissive materials such as polyamides, polyesters, polyethylene, acrylic, glass, or similar materials. In one variation, a protective cover may comprise optically clear polyester.

Test strip (311) also comprises a sample pad (or sample application band) (307) that is in fluid communication with contact band (306), such that a fluid sample applied to sample pad (307) is directed to contact band (306). As shown in FIG. 3D, sample pad (307) may be positioned so that it at least partially overlaps contact band (306). Other appropriate arrangements may also be used. Sample pad (307) has a width (L_{SP}) that may be, for example, from about 6 mm to about 20 mm (e.g., 10 mm or 14 mm), and contact band (306) has a width (L_{CB}) that may be, for example, from about 4 mm to about 15 mm (e.g., 5 mm, 7 mm, 8 mm, or 10 mm). Additionally, the overlap interface between sample pad (307) and contact band (306) has a width (L_{IF}) that may be, for example, from about 3 mm to about 8 mm. In other variations, the sample pad may overlap the entire width of the contact band such that the contact band is disposed between the sample pad and the backing strip. Alternatively, the sample pad and the contact band may both be in direct contact with the backing strip and arranged such that an edge of the sample pad is in fluid contact with an edge of the contact band (e.g., end-to-end).

Substrate (302) may comprise any appropriate material or materials. In general, substrate (302) may comprise one or more relatively robust materials through which a fluid sample may easily travel. Typically, substrate (302) may be made of any material or materials having sufficient porosity to allow fluid flow along the surface of the substrate and through its interior by any of a variety of mechanisms, such as capillary action. For example, a substrate may have sufficient porosity to allow movement of particles such as analyte-binding agents and/or analytes. It may also be desirable for a substrate to be wettable by the fluid in the sample to be tested. For example, a hydrophilic substrate may be used for aqueous fluids, while a hydrophobic substrate may be used for organic solvents. Hydrophobicity of a membrane can be altered to render the membrane hydrophilic for use with aqueous fluid, by processes such as those described in U.S. Pat. Nos. 4,340,482 or 4,618,533, which describe transformation of a hydrophobic surface into a hydrophilic surface. Non-limiting examples of materials which may be suitable for use in substrate (302) include cellulose, nitrocellulose, cellulose acetate, glass fiber, microfibers, nylon, polyelectrolyte ion exchange membranes, acrylic copolymer/nylon, and polyethersulfone.

In some variations, a test strip may be formed by joining together different portions or sections of a substrate or multiple different substrates. In certain variations, a test strip may be in the form of a continuous, integral strip. In other variations, multiple strips may be overlapped with and/or connected to each other, so that a fluid applied on one strip may flow to the other strips. In some variations, a substrate may comprise a gel such as a cross-linked polymer (e.g., polyacrylamide) or agarose. A cross-linked polymer substrate may be synthesized with a desired gel pore size, which may depend, for example, on the size of the control analyte and/or the target analyte. In certain variations, microchannels may be formed in a substrate (e.g., to urge and guide fluid travel at a particular direction and/or speed).

Contact band (306) comprises a target analyte binding agent and a control analyte. The control analyte may be any compound that does not bind (or is not bound by) anything that may be in the sample. In some variations, the control analyte may comprise dinitrophenol conjugated to BSA (bovine serum albumin). Target analyte binding agents include moieties (or compositions) that recognize and bind an analyte. However, in some variations, the analyte binding agent may non-selectively bind any analyte. Exemplary target analyte binding agents include, but are not limited to, antibodies, antigens, peptides, haptens, engineered proteins, and other protein-binding reagents, such as nucleic acids (e.g., RNA, DNA, PNA, and other modified nucleic acids), and aptamers, as well as other biological and chemical molecules. An antibody may include an antibody binding region, complementarity determining regions (CDR), single chain antibody, chimeric antibody, or humanized antibody. An antibody may be a monoclonal antibody or a polyclonal antibody.

Contact band (306) typically has an upper surface and a lower surface, and in one variation, the lower surface of the contact band may be in fluid contact (e.g., capillary contact) with substrate (302). Certain variations of contact band (306) may comprise a target analyte binding agent and a control analyte, each labeled with a different detectable marker. The detectable marker attached to the target analyte binding agent and/or the control analyte may comprise any of a wide variety of materials, so long as the marker can be detected. The quantity/concentration of the target analyte binding agent and the control analyte may vary relative to each other, or for different target analyte binding agents. In some variations, the target analyte binding agent and the control analyte may not be applied directly to the test strip, but may be added to the sample before or after the sample is applied to the test strip.

In some cases, at least one of the target analyte binding agents and/or control analytes may be conjugated with a fluorophore that allows for detection via fluorescence upon application of light from a light source. Generally, in such cases, each of the different target analyte binding agents and/or control analytes will be conjugated with a different fluorophore. For example, a test strip may comprise a band comprising a target analyte binding agent conjugated with a first fluorophore, and a control analyte conjugated with a second fluorophore that is different from the first fluorophore. The fluorophores may be selected to fluoresce at different wavelengths (upon application of light from a light source, such as a laser), such that they can be used to detect and distinguish the target analyte binding agent and the control analyte. Examples of fluorophores which may be suitable here include HiLyte Fluor™ 647 fluorophore (AnaSpec) and DyLight-800 fluorophore (ThermoScientific), or any other appropriate commercially available or proprietary fluorophore, such as any dye in the cyanine family (Jackson ImmunoResearch), or the Alexa Fluor family of dyes (Invitrogen-Molecular Probes). In some variations, the target analyte or control analyte may be directly bound by a fluorophore.

While fluorophores have been described as detection agents, some variations of test strips may use other types of detection agents and methods. For example, additional detection methods based on absorption, reflectance, luminescence (e.g., chemiluminescence), or electrical applications may be employed. In certain variations, detection may be indicated by a change in color (or, in some cases, a lack of change in color) in one or more zones of a test strip or other testing substrate or medium. In some variations, detection may be indicated by a change in pH, where the detector function as a pH color indicator. In certain variations, the presence or absence of a specific chemical moiety may be used for detection. In some variations, functionalized carbon nanotubes may be used as Raman labels, and surface-enhanced Raman spectroscopy (SERS) may be used for detection. Additional description of detection methods employing carbon nanotubes is provided, for example, in Srivastava, S. & J. LaBaer, “Nanotubes Light Up Protein Arrays,” Nature Biotechnology, Vol. 26, No. 11 (November 2008) 1244-1246, and in Chen et al., “Protein Microarrays with Carbon Nanotubes as Multicolor Raman Labels,” Nature Biotechnology, Vol. 26, No. 11 (November 2008) 1285-1292. Additional examples of detectable markers include, but are not limited to, particles, luminescent labels (e.g., chemiluminescent labels), calorimetric labels, chemical labels, enzymes, radioactive labels, radio frequency labels, and metal colloids. Further examples of common detection methodologies include, but are not limited to, optical methods (e.g., measuring light scattering, using a luminometer, photodiode or photomultiplier tube), radioactivity (measured with a Geiger counter, etc.), electrical conductivity or dielectric (capacitance), and electrochemical detection of released electroactive agents (e.g., indium, bismuth, gallium or tellurium ions, as described by Hayes et al. (Analytical Chem. 66:1860-1865 (1994)), or ferrocyanide, as suggested by Roberts and Durst (Analytical Chem. 67:482-491 (1995)), wherein ferrocyanide encapsulated within a liposome is released by the addition of a drop of detergent at the detection zone with subsequent electrochemical detection of the released ferrocyanide). Other methods may also be used, as appropriate. Moreover, a single detection method may be used, or multiple (e.g., two, three) different detection methods may be used together.

In certain variations, a contact band such as contact band (306) may comprise more than two different target analyte binding agents, such as three, four, five, or ten different target analyte binding agents, so that the same strip may be used to evaluate for multiple different diseases or indications. Similarly, some systems may employ multiple different test strips, with each individual strip testing for a different disease or indication. Certain variations of systems may test for 10 to 20 analytes, for example.

In some variations, a test strip may comprise a buffer region, optionally comprising a buffer pad, to which buffer is added. The buffer pad may have an upper surface and a lower surface, where the lower surface of the buffer pad may be in capillary contact with the test strip substrate. The buffer region may be located at or near the contact band or conjugate pad of the test strip. When buffer is added to the test strip, the buffer may dissolve the target analyte binding agent and control analyte in the contact band, and may flow along the test strip until it reaches the sample detection band and/or wicking portion, for example.

Sample detection band (308) may comprise at least one analyte capture agent. Capture agents are specific types of analyte binding agents that are immobilized on the test strip, and may comprise a moiety (or composition) that recognizes and selectively binds to the target analyte. When a capture agent binds to an analyte, the analyte is “captured” on the test strip. In some variations, the analyte may be bound to another analyte binding agent, prior to binding to the capture agent. In other variations, the capture agent may not be selective for the target analyte, and may non-specifically bind analytes. The quantity/concentration of an analyte capture agent and a control analyte capture agent on a test strip may vary relative to each other. Moreover, the quantity/concentration of different analyte capture agents having different binding properties may vary.

In some variations, sample detection band (308) may comprise a target analyte capture agent and a control analyte capture agent. The target analyte capture agent may be configured to bind to the target analyte binding agent or to the target analyte. Similarly, the control analyte capture agent may be configured to bind to the control analyte. In some variations in which the test strip comprises a target analyte binding agent, or in which a target analyte binding agent is pre-mixed with the sample before the sample is added to the test strip, there may be at least two agents that bind the target analyte—one that is detectably labeled and one or more capture agents that are immobilized in the sample detection band. It is noted that at least one of the agents that bind the target analyte should bind only to the target analyte and not to any of the other components in the sample (i.e., the agent should bind the target analyte selectively or specifically). In one variation, the one or more capture agents that are immobilized in the sample detection band may be target analyte specific/selective and the target analyte binding agent that is labeled with a detectable marker may be capable of binding non-selectively to the target analyte. In another variation, the one or more capture agents that are immobilized in the sample detection band may be capable of binding non-selectively to the target analyte and the target analyte binding agent which is labeled with a detectable marker may be target analyte specific/selective. In yet another variation, both the capture agent(s) and the detectably labeled target analyte binding agent may be target analyte specific/selective.

Non-limiting examples of target analyte capture agents which may be appropriate for use here include antibodies, engineered proteins, peptides, haptens, lysates containing heterogeneous mixtures of antigens having analyte binding sites, ligands, nucleotides, nucleic acids, aptamers, and receptors.

Control analyte capture agents are generally selected so as to bind specifically to molecules other than molecules that specifically bind to the target analyte. A control analyte capture agent may be a compound that does not bind to anything that might be present in the sample. Substances useful as control analyte capture agents include those substances described above as useful as target analyte capture agents. In some variations, a control analyte capture agent may be a naturally occurring or engineered protein. A control analyte and its corresponding control analyte capture agent may also be a receptor-ligand pair. Additionally, either a control analyte or its corresponding control analyte capture agent may be an antigen, another organic molecule, or a hapten conjugated to a protein non-specific for the analyte of interest (the target analyte). Descriptions of other suitable variations of control analytes and/or control analyte capture agents are described, for example, in U.S. Pat. No. 5,096,837, and include IgG, other immunoglobulins, bovine serum albumin (BSA), other albumins, casein, and globulin. In some variations, a control analyte capture agent may comprise a rabbit anti-dinitrophenol (anti-DNP) antibody that binds to dinitrophenol conjugated to BSA. Additional beneficial characteristics of control analyte capture agents include, but are not limited to, stability in bulk, non-specificity for the target analyte, reproducibility and predictability of performance in test, molecular size, and avidity of binding to the control analyte.

In some variations, a capture agent, such as a target analyte capture agent or a control analyte capture agent, may be any macromolecule that specifically binds its target with high affinity, and that also includes subsidiary groups that may, for example, be used to attach a detector probe or detection agent.

In some variations, a sample detection band may comprise different capture agents that are each tagged with a different detectable marker. The markers may be activated (i.e., such that they become detectable) only upon the capture of the intended analyte. For example, the target analyte capture agent may be tagged with one fluorescent marker, while the control analyte capture agent may be tagged with a different fluorescent marker, where the fluorescence of each marker is only activated upon analyte binding. Examples of fluorescent markers and other detectable markers that may be used include those described herein.

Of course, while a test strip including a target analyte capture agent and a control analyte capture agent is described here, some variations of test strips may include more than one (e.g., three, four, five, or ten) target analyte capture agent and/or control analyte capture agent. Additionally, certain variations of test strips may not include a control analyte capture agent in the same location as a target analyte capture agent.

Wicking portion (310) may be formed of an absorbent substance that can absorb the sample fluid and/or buffer. The absorption capacity of wicking portion (310) may be sufficiently high to allow the wicking portion to absorb the fluid or fluids that are delivered to the test strip. Examples of substances suitable for use in a wicking portion include cellulose and glass fiber.

During use of test strip (300), a fluid sample may be applied to contact band (306) in the direction of arrow (A1) (e.g., via first port (202) of cartridge (111)). The sample may be any suitable fluid sample (e.g., a biological sample such as a bodily fluid) that is likely to contain the analyte of interest. For example, the fluid sample may be a blood, plasma, serum, saliva, mucus, urine, cervical mucus, semen, vaginal secretions, tears, or amniotic fluid sample. In some variations, the fluid sample may be a whole blood sample. In certain variations, the fluid sample may not be a biological sample, but may be a fluid in which, for example, impurities or contaminants are to be detected. The sample may (but need not) be treated prior to being deposited on the test strip. As an example, in some variations, one or more amplification agents and/or preservatives may be added to the fluid sample prior to its addition to the test strip. As another example, in certain cases in which the sample is too viscous to flow evenly on the test strip, the sample may be pre-treated with one or more agents that reduce the viscosity of the fluid, including, but not limited to, one or more mucolytic agents or mucinases. Additionally, in some cases, the fluid sample may be passed through one or more filters prior to being applied to the test strip. For example, when the fluid sample is a blood sample, the fluid sample may be passed through one or more filters that retain blood cells but that allow the fluid itself to pass through. When a fluid sample is added to the test strip, it dissolves the target analyte binding agent and the control analyte in contact band (306).

Referring to FIG. 3B, after the fluid sample has been applied to the test strip, the target analyte binding agent and the control analyte may be solubilized/dissolved, and the target analyte present in the sample may bind to the target analyte binding agent. Both the target analyte binding agent (which may be bound to any target analyte that is present in the sample) and the control analyte may travel along substrate (302) in the direction of arrow (A2) (e.g., as a result of capillary action, the effect of the wicking portion (310), or any directional field, such as an applied magnetic or electrical field, and/or gravitational field).

A target analyte may be any compound for which a specifically binding agent naturally exists or can be prepared. The term “analyte” may refer to both free/un-complexed analyte as well as to analyte that is bound by one or more analyte binding agents that may, or may not, be detectably labeled. Examples of classes of analytes include, but are not limited to, proteins, such as hormones and other secreted proteins, enzymes, and cell surface proteins; glycoproteins; peptides; small molecules; polysaccharides; antibodies (including monoclonal or polyclonal antibodies); nucleic acids; drugs; toxins; viruses or virus particles; portions of a cell wall; and other compounds possessing epitopes. Typically, an analyte may be any molecule (e.g., large or small) that specifically binds to a capture reagent with high specificity, and that is capable of binding to a detector probe or detection agent, or specifically to a molecule containing the detector probe or detection agent.

Any number of different types of analytes may be detected and/or measured using the devices, systems, and methods described here. Exemplary analytes which may be evaluated here include alanine aminotransferase, albumin (plasma), albumin (urine), amakacin, amitriptyline, amylase, aspartate aminotransferase, bilirubin, Brain Natriuretic Peptide (BNP), calcitonin (hCT), cancer chemotherapeutic agents, carbamazepine, Cardiac Troponin I (cTn1), cholesterol (HDL), cholesterol (LDL), cholesterol (total), Chorionic Gonadotropin (hCG), cortisol, C-Reactive Protein (CRP), creatine, creatine kinase (activity), Creatine Kinase Isoenzyme MB (CKMB), creatinine (blood), creatinine (urine), digoxin, estradiol, estriol (free & total), estrogens (total), α₁-Fetoprotein (AFP), Follicle Stimulating Hormone (hFSH), gentamycin, glucagon, glucose, haptoglobin, HbAlc, hemoglobin, homocysteine, kanamycin, Lactate Dehydrogenase (LDH; lactate→pyruvate), lithium, Luteinizing Hormone (hLH), myoglobin, nortriptyline, paraquat, Parathyroid Hormone (hPTH), phenobarbital, phenytoin (diphenylhydantoin), phosphatase (acid), phosphatase (alkaline) (ALK-P), potassium, progesterone, Prostate Specific Antigen (PSA), protein (total), rennin, sodium, somatotropin (hGH), testosterone, theophylline, thyroid microsomal antibodies, Thyroid Stimulating Hormone (hTSH), thyroxine (T4), transferrin, triglycerides, triiodothyronme (T3), urea nitrogen, uric acid, valproic acid, vancomycin, vitamins and nutrients, and warfarin (coumadin). These are only exemplary analytes, and other analytes may be detected and evaluated using the systems described here. For example, any analyte that may be present in a fluid for which an antibody (or aptamer or nucleic acid or nucleotide specifically binding to a protein or to an analyte) may be developed may be evaluated using the diagnostic systems described here. In some variations, the devices, systems, and methods described here may be used to detect physiological markers related to cancer, cholesterol levels, allergies, nephrology, the immune system, the endocrine system, heme levels, cardiac diseases, blood gas, urinalysis, and various infectious diseases.

As the fluid sample passes over contact band (306), the target analyte will bind to the target analyte binding agent to form a target analyte complex. As described previously, the target analyte complex and the control analyte may be tagged with a detectable marker, such as a fluorescent marker. Referring now to FIG. 3C, the target analyte complex and control analyte will travel along substrate (302) in the direction of arrow (A2), and will eventually contact sample detection band (308), where the target analyte capture agent will bind the target analyte complex and/or the target analyte. Additionally, the control analyte capture agent may bind the control analyte. In some variations, the binding of the target analyte complex by the target analyte capture agent, as well as the binding of the control analyte by the control analyte capture agent, may activate the detectable markers.

Once the target analyte complex and the control analyte have reached sample detection band (308), the appropriate action may be taken to detect the target analyte or analytes that were present in the fluid sample and that are now bound to the target analyte capture agent or agents. Here, such detection will be described in terms of application of lasers or other light sources to detect fluorescence of the conjugated fluorophores. However, as discussed above, other detection methods may also be used, as appropriate. Application of the lasers or other light sources to the fluorophores, when of the appropriate wavelength, may activate the fluorophores and cause them to fluoresce. Here, the amount of target analyte and control analyte that are present may be evaluated based on relative fluorescence intensity. The ratio of the fluorescence intensity of the target analyte to the control in the same band may be indicative of the concentration of the target analyte in the sample or may be used to reduce variability of measured intensity.

As discussed in further detail below, by locating the control analyte capture agent and the target analyte capture agent in the same location on the test strip (i.e., sample detection band (308)), measurement variability (e.g., resulting from membrane differences, coating condition differences, viscosity differences, sample addition differences, etc.) may be reduced, in some cases significantly.

As previously described, control analytes may be provided at contact band (306), and control analyte capture agents may be provided at sample detection band (308). The control analyte capture agents may bind the control analytes (which may be dissolved in a fluid sample traveling across test strip substrate (302)). Such a control binding pair (i.e., a control analyte and its corresponding control analyte capture agent) may act as an internal control. Internal control mechanisms, which are described in more detail below, may help compensate for strip-to-strip variability to ensure a precise and accurate analyte reading.

As described above, a control analyte capture agent and a target analyte capture agent may be located in the same band on a test strip. Co-localization of the control analyte capture agent and the target analyte capture agent may ensure that both capture agents are exposed to the same physical, environmental, and chemical conditions after manufacturing. Moreover, to ensure that the control analyte capture agent and the target analyte capture agent are subject to the same conditions during the manufacturing process, these capture agents may be synthesized and handled in the same batch, and applied to the test strip at the same time. Such treatment and arrangement of the control analyte and target analyte capture agents may act to normalize target analyte binding with respect to control analyte binding to remove any manufacturing and environmental variability that may impact analyte binding. Identical treatment and application of the control analyte and target analyte capture agents to the test strip may thereby allow for precise and accurate readings (i.e., providing for more effective normalization against any systemic variability for a more precise measurement). Similarly, the target analyte binding agent and the control analyte may be manufactured, handled, and applied to the contact band under identical conditions, and the same precision and accuracy results may occur. Examples of manufacturing variabilities include temperature differentials between different locations on a test strip, agent quantity dispense differentials, differentials occurring when agents are applied to a test strip at two different time points, and agent density differentials when agents are applied to a test strip under different circumstances (e.g., agent viscosity, different application methods, different wash steps). Examples of environmental variability include humidity and temperature differentials, strip handling pattern, exposure pattern to target analyte and control analyte and such similar factors.

### Methods of Making a Test Strip, Cartridge, and Cartridge Kit

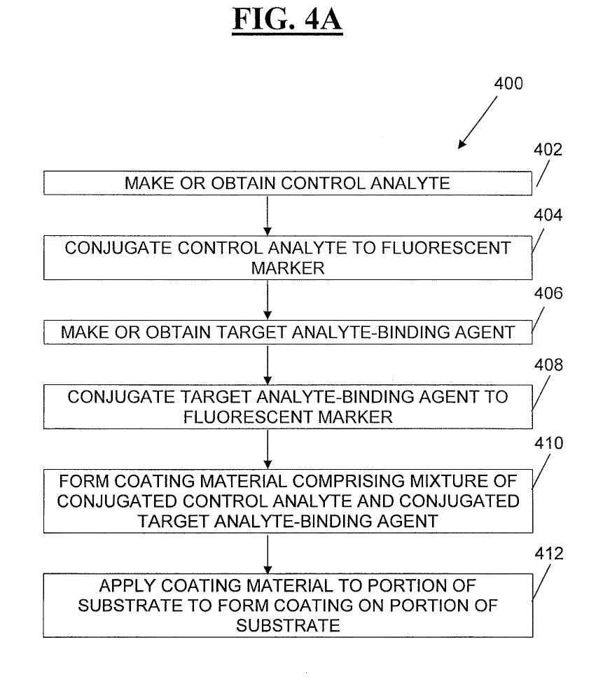

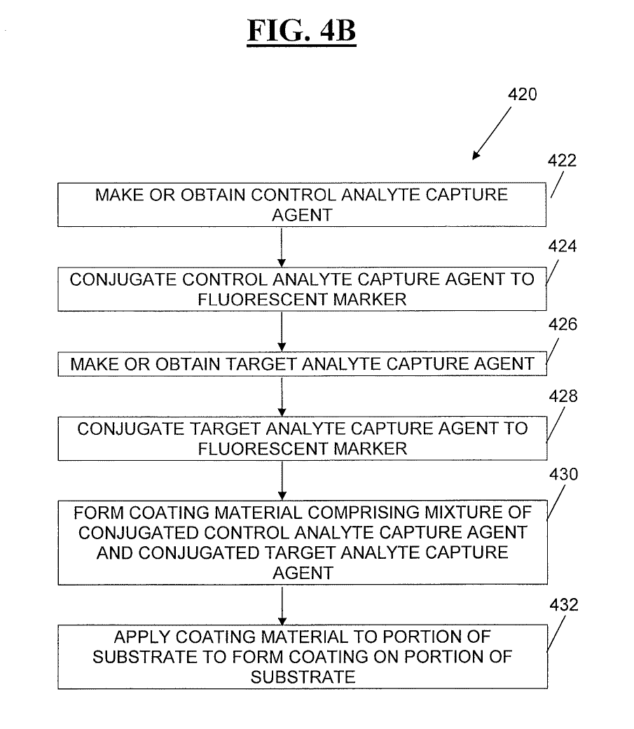

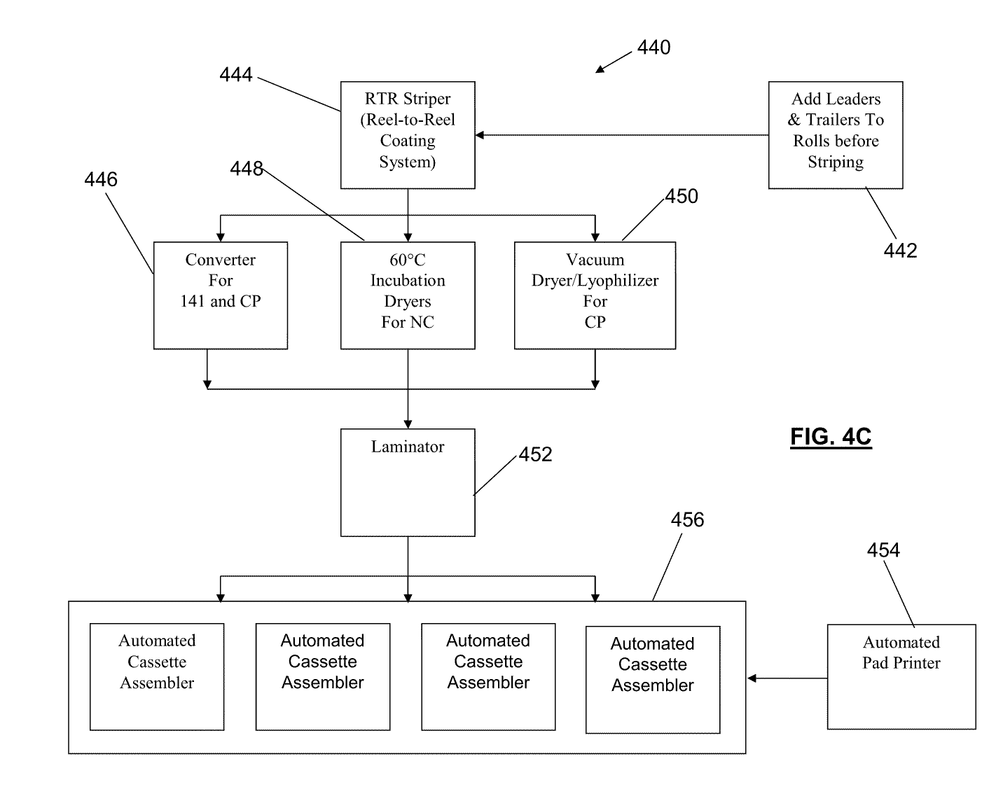

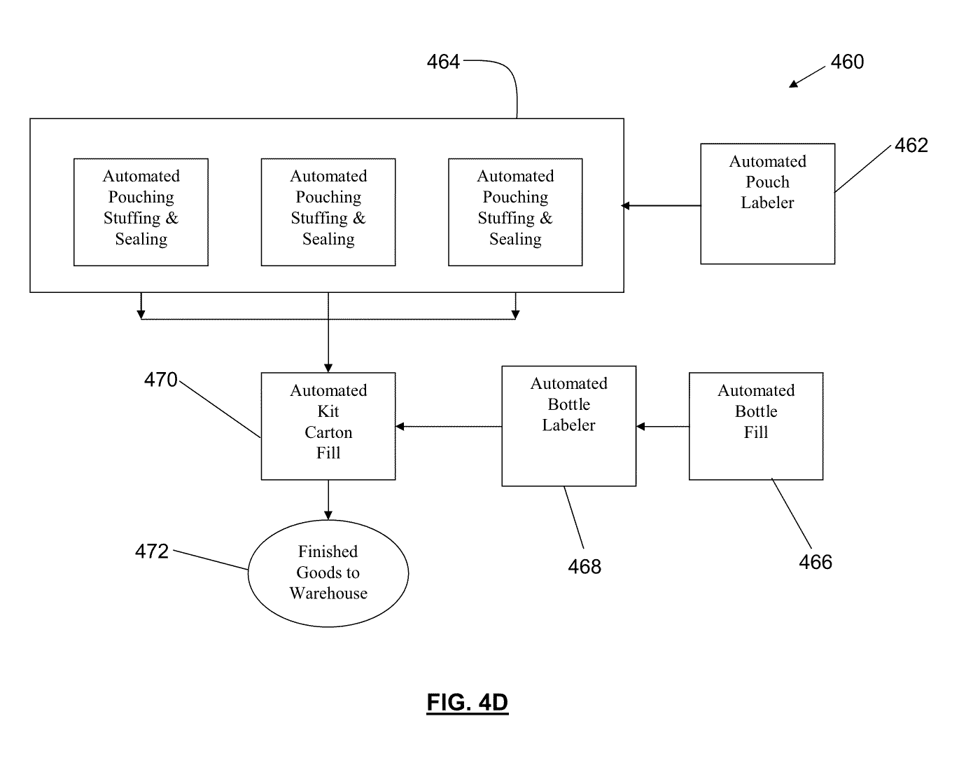

FIG. 4A is a flowchart representation of a variation of a method (400) for making contact band (306), in cases in which contact band (306) comprises a target analyte binding agent and a control analyte. As shown there, method (400) comprises making or obtaining the control analyte (402), conjugating the control analyte to a fluorescent marker (or fluorophore) (404), making or obtaining the target analyte binding agent (406), conjugating the target analyte binding agent to a fluorescent marker (or fluorophore) (408), forming a coating material comprising a mixture of the conjugated control analyte and the conjugated target analyte binding agent (410), and applying the coating material to a portion of a substrate to form a coating on the portion of the substrate (412).

In other variations of a detection system, the capture agents on the sample detection band (308) may be tagged with fluorescent markers that are activated (i.e., detectable) only when the capture agents bind their intended analytes. FIG. 4B is a flowchart representation of a variation of a method (420) for making a test strip having a sample detection band comprising a co-localized target analyte capture agent and control analyte capture agent. As shown there, method (420) comprises making or obtaining the control analyte capture agent (422), conjugating the control analyte capture agent to a fluorescent marker (or fluorophore) (424), making or obtaining the target analyte capture agent (426), conjugating the target analyte capture agent to a fluorescent marker (or fluorophore) (428), forming a coating material comprising a mixture of the conjugated control analyte capture agent and the conjugated target analyte capture agent (430), and applying the coating material to a portion of a substrate to form a coating on the portion of the substrate (432). While certain variations of methods of making test strips have been described, other variations of methods may also be used, as appropriate. Similarly, any suitable method of making a test strip-retaining cartridge may be used. For example, FIG. 4C is a flowchart representation of a variation of a method (440) for making a cartridge for retaining a test strip. As shown there, method (440) comprises adding leaders and trailers to rolls (442), and striping the rolls using a reel-to-reel coating system (444). The leaders and trailers that are added to the rolls are usually plastic tap, which may be added to the first and last edge of a roll to save the actual roll material, such cellulose and glass fiber, prior to coating. A portion of the rolls designated for a sample pad and the contact band (or conjugate pad) may be converted (446), a portion of the rolls designated for nitrocellulose may be incubated at 60° in dryers (448), and a portion of the rolls designated for the contact band (or conjugate pad) may be subjected to vacuum drying or lyophilization (450). In some variations, a whole coated roll may be placed in a vacuum and dried or freeze-dried. After these processes, the rolls may be laminated (452). Printed pads may be made or acquired (454), and may be assembled with portions of the rolls to form cassettes (456).

In some variations, multiple cartridges may be automatically assembled together into a kit. In other variations, the kit may be manually assembled. For example, FIG. 4D is a flowchart representation of a variation of a method (460) for assembling a test cartridge strip and packing it into a kit. As shown there, method (460) comprises making or acquiring labeled pouches (462), and sealing cartridges (that are made and/or acquired) into the labeled pouches (464). Additionally, method (460) comprises filling (466) and labeling (468) bottles that are made and/or acquired. The pouches may then be placed into cartons with labeled bottles (470) and stored (472), for example, in a warehouse.

### Optical Module

As discussed above, a detection system, such as system (100) or system (120), may be used to detect and evaluate analytes in a test strip, such as test strip (300) or test strip (311). Components of detection systems, such as detection systems (100) and (120), will now be described in additional detail.

As described above, some variations of POC diagnostic systems evaluate the presence of one or more analytes in a fluid sample using a light-based detection mechanism. For example, target and/or control analytes may be tagged with one or more fluorescent markers, where the markers may be activated by light (e.g., light within their excitation spectrum), and fluoresce within their emission spectrum. A diagnostic system may have an optical module comprising an excitation module that emits laser beams within the excitation spectrum of the fluorophore to activate the fluorescent markers. The optical module may also comprise a detection module that is configured to detect fluorescent light within the emission spectrum of the fluorescent markers. The intensity of the fluorescent emission may be qualitatively and/or quantitatively analyzed to determine the presence and/or concentration of the target analyte(s).

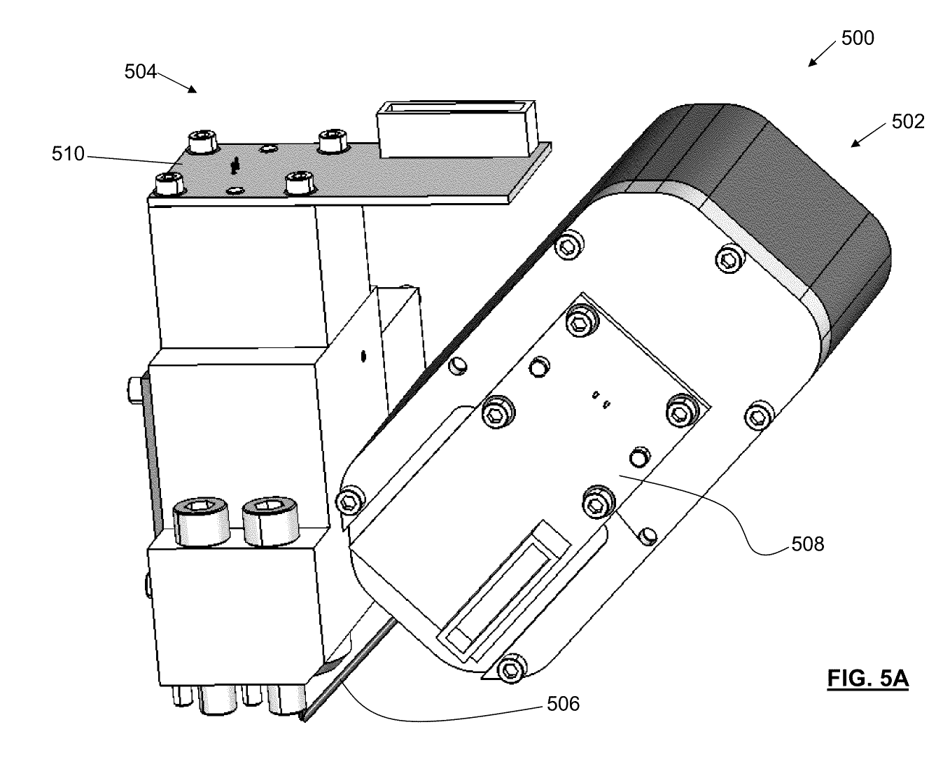

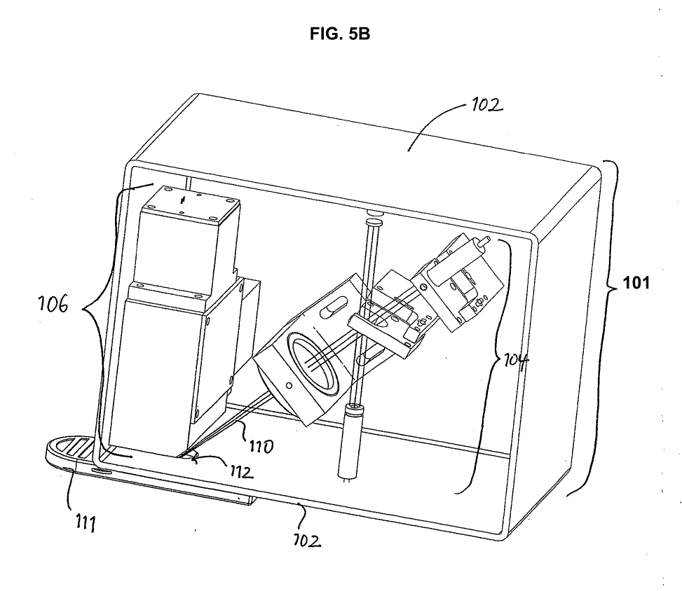

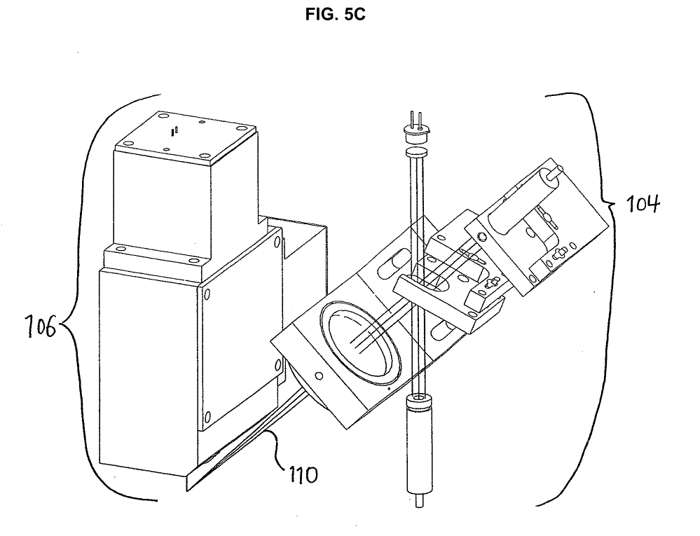

One example of an optical module (500) is shown in FIG. 5A. As shown there, optical module (500) comprises an excitation module (502) and a detection module (504). Excitation module (502) may be arranged to direct a laser beam (506) to a test strip retained within a test cartridge (not shown). For example, laser beam (506) may be directed to a location on the test strip that is within the detection range of detection module (504). Laser beam (506) may be a single wavelength light, or may have a variety of wavelengths that are in the excitation spectra of the test strip fluorophores. According to the emission spectra of the fluorophore(s), detection module (504) may have one or more optical elements, such as filters, dichroic mirrors, etc. to capture the emitted light.

An optical module may comprise one or more light sensor boards. For example, excitation module (502) may comprise a light sensor board (508), which may be used to monitor the power of laser beam (506). This may allow for more precise control of the laser beam (e.g., by normalizing every laser beam pulse). Alternatively or additionally, detection module (504) may comprise a light sensor board (510), which may be used to detect the intensity of the light emitted from the fluorescent tags. An optical module may have any number of light sensor boards as needed for detecting the intensity of the light (i.e., excitation and/or emitted light) within the optical module and/or from a test strip. For example, an optical module may have 3, 4, 5, 10, etc. light sensor boards.

FIGS. 5B and 5C show optical module (101) of system (100) (FIG. 1B) in enlarged detail. FIG. 5B shows optical module (101) including housing (102), as well as cartridge (111) for reference, while FIG. 5C shows the inner components of optical module (101), and thus excludes housing (102). As shown in FIGS. 5B and 5C, optical module (101) comprises detection module (106) and excitation module (104). During use, excitation module (104) directs laser beams (110) to a sample in cartridge (111), and detection module (106) detects the resulting fluorescence. The various components of these two modules will be discussed in further detail below.

While FIGS. 5B and 5C show one configuration of an optical module where detection module (106) and excitation module (104) are separate entities, other suitable variations of optical modules may also be used. For example, other variations of optical modules may include detection and excitation components that are more integrated, rather than being modularized. Moreover, while an optical module comprising one detection module and one excitation module has been described, in some variations an optical module may comprise multiple detection modules or components, and/or multiple excitation modules or components. As an example, an optical module may include multiple pairs of excitation and detection modules or components, with each pair configured for use with one or more specific types of fluorophores having different excitation and emission spectra.

Certain variations of optical module (101) may provide for access to one or more of the optical module's internal components. Such access may, for example, allow for adjustment of certain component parameters, such as the distances between the various components, aperture size of lenses and/or condensers, and the angles of reflecting mirrors and other filters. Access to adjust these parameters may be provided, for example, through apertures in housing (102), and/or via electrical and/or mechanical interfaces to one or more external controllers that actuate the various internal components. Additionally, other variations of optical modules may utilize different configurations of excitation modules, such as those described below.

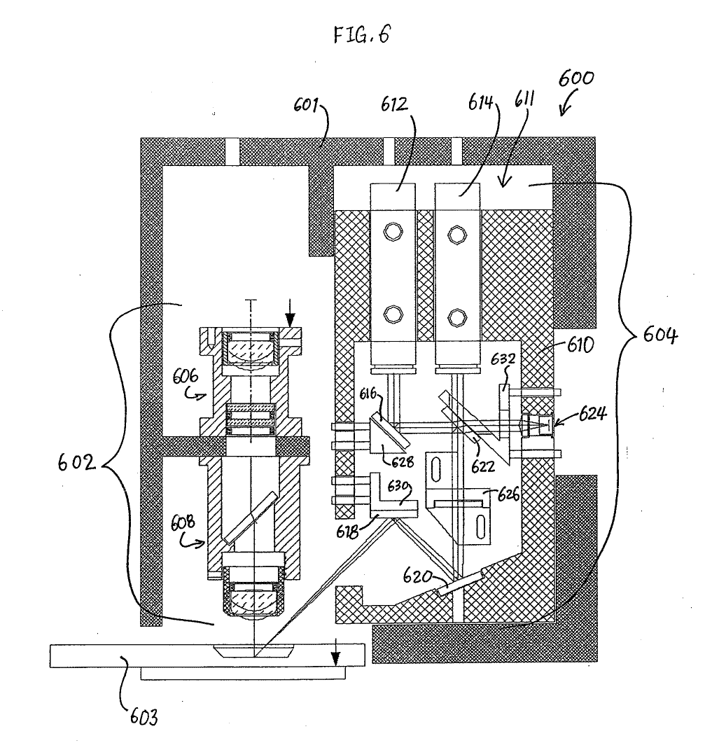

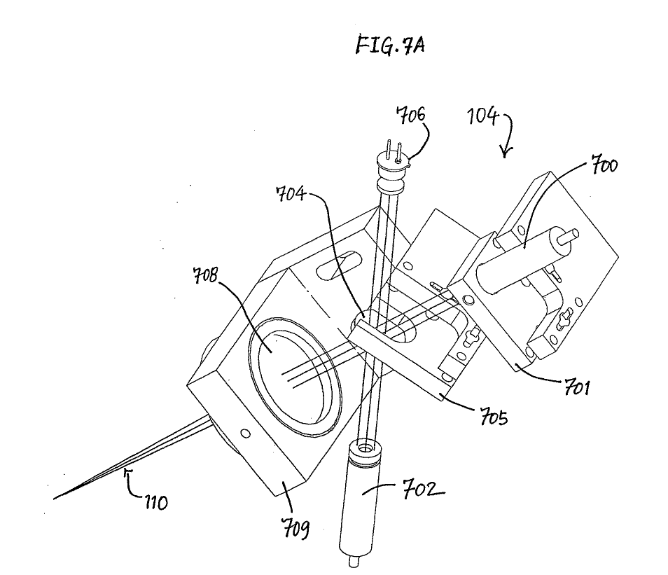

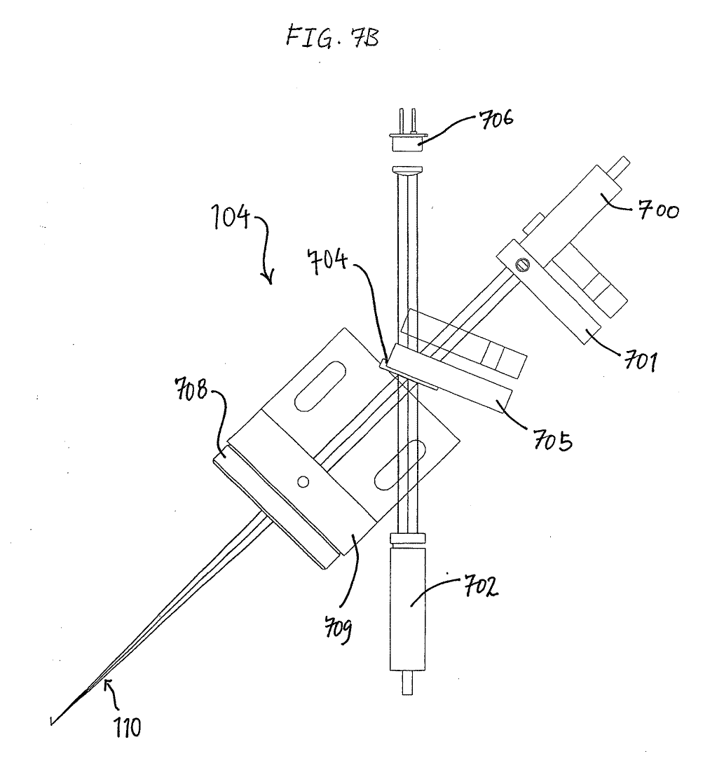

FIG. 6 depicts another variation of an optical module (600), with a cartridge (603) included as a frame of reference. As shown in FIG. 6, optical module (600) comprises a housing (601) containing a detection module (602) and an excitation module (604). While housing (601) is depicted as having a certain configuration, other suitable housing configurations may be employed. For example, a housing may have relatively little extra space, such that the housing is essentially fitted to its internal components. Housing (601), as well as any of the other housings described here, may be made of any suitable material or materials including, for example, polymers, metals, and metal alloys (e.g., aluminum alloys, stainless steel, etc.).

Detection module (602) comprises two detector units (only one of which—detector unit (606)—is shown) and an objective lens unit (608). Excitation module (604) comprises a housing (610) that is used to help contain and/or position the various components of the excitation module, and that is positioned within a space (611) of housing (601) of optical module (600). As shown in FIG. 6, the components of excitation module (604) include two lasers (612) and (614), two adjustable mirrors (616) and (618), a stationary minor (620), a dichroic filter (622), a photodiode (624), and a cylindrical lens (626). Adjustable mirrors (616) and (618) are mounted to adjustable mirror mounts (628) and (630), respectively, and dichroic filter (622) is mounted to an adjustable mount (632). Cylindrical lens (626) is positioned over mirror (620) so that beams may be focused in a narrow line and reflected by mirrors (620) and (618) to excite the sample contained in cartridge (603). Exemplary detection modules and excitation modules will now be discussed in additional detail.

### Excitation Module

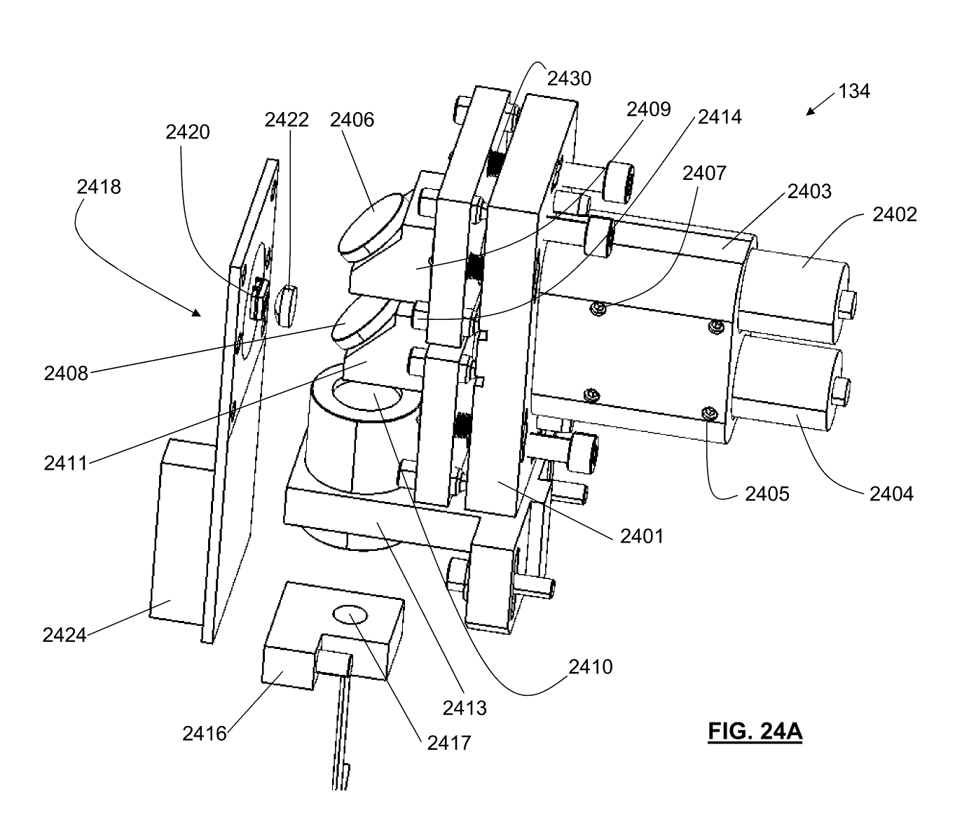

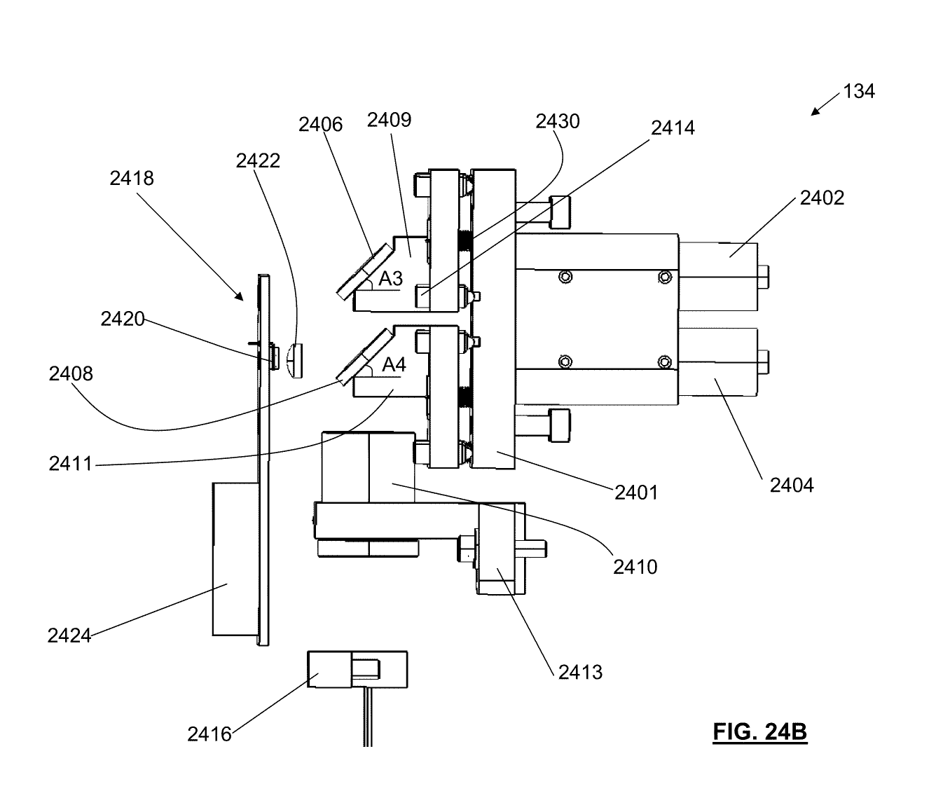

Any suitable configuration of an excitation module may be used in the devices described herein. One exemplary excitation module is excitation module (134) of optical module (130) (FIG. 1A). Excitation module (134) is shown in enlarged detail in FIGS. 24A and 24B. As shown there, excitation module (134) comprises a first laser (2402) configured to emit a first laser beam with a first spectral distribution, and a second laser (2404) configured to emit a second laser beam with a second spectral distribution. Excitation module (134) also comprises one or more optical components arranged to combine and focus the first and second laser beams into a single beam that is directed at a single location (e.g., at a location that intersects with an optical axis of an objective lens of a detector module). The lasers and optical components may be adjustably or fixedly attached to a base plate (2401). While excitation module (134) comprises two lasers, other variations of excitation modules may comprise one, three, four, six, etc. lasers, according to the number of unique wavelengths of light needed for detecting the desired number of target and/or control analytes.

First laser (2402) may comprise a laser diode that emits laser light in the infrared range (e.g., 780 nanometers (nm)) and/or second laser (2404) may comprise a laser diode that emits laser light in the red range (e.g., 635 nm). The power and/or pulse width of each laser emission may be electronically or computer controlled. First laser (2402) may emit light with an output power from about 5 milliwatts (mW) to about 35 mW (e.g., 30 mW), and/or second laser (2404) may emit light with an output power from about 3 mW to about 25 mW (e.g., 20 mW). The light emitted by the first and second lasers may also be frequency modulated. Various laser pulse modifications will be described further below.

First and second lasers (2402) and (2404) may be retained by a laser mount (2403) attached to base plate (2401), and are arranged such that the laser beams they emit are collimated (i.e., substantially parallel). However, in other excitation modules, lasers may be arranged such that their laser beams are not parallel but are at an angle (e.g., perpendicular). Lasers (2402) and (2404) may have an alignment ring that may be adjusted to collimate the beams of laser (2402) with the beams of laser (2404). Once the beams of the first and second lasers are collimated and/or aligned as desired, the alignment ring may be secured using an adhesive, such as Loctite® 271 Threadlocker-Red adhesive. Collimation of the two laser beams may be achieved by adjusting the laser-embedded laser lens, which may be an integral part of a typical laser diode module.

The laser diodes may emit laser beams that are circular, oblong, rectangular, etc. The orientation of a laser beam may be adjusted by physical rotation of the laser diode and/or by controlling the beam position using a laser beam profiler. A manufacturing jig may be used to precisely position the laser diode as desired. For example, the laser diode emitting an elliptical beam may be positioned such that the long axis of the elliptical beam is oriented so that the beam focused by the cylindrical lens creates a line that may be parallel to the sample bands in the cassette. In some variations, the locations of the lasers may be fixed with respect to each other and/or the other optical components, while in other variations, the locations of the lasers may be adjustable. For example, first laser (2402) and second laser (2404) may be slidably and/or rotatably retained by laser mount (2403), or they may be fixedly retained by laser mount (2403). In some variations, the lasers may be movable with respect to the mount, while the other laser is fixed with respect to the mount. The position and orientation of second laser (2404) within laser mount (2403) may be secured by one or more set screws (2405), while the position and orientation of first laser (2402) may be secured by one or more mounting screws (2407). Other fixation mechanisms may also be used.

The laser beams or other light sources of the systems described here may follow any appropriate path during use. In some variations, the light path of laser beams may be directed by one or more optical components. For example, the optical components may be arranged to combine and focus first and second laser beams into a single beam that is directed at a location that intersects with an optical axis of an objective lens of a detector module of the system. For example, as depicted in FIGS. 24A and 24B, excitation module (134) comprises a mirror (2406) configured to reflect the beam from first laser (2402), a dichroic reflector (2408) configured to reflect the beam from second laser (2404) and transmit the beam from first laser (2402), and a cylindrical lens (2410) configured to focus the beams from the first and second lasers to a single location. As shown, minor (2406) is secured on a mirror mount (2409) in front of laser (2402), such that the reflective surface of minor (2406) directs the laser beam at an angle (A3) (FIG. 24B) towards dichroic reflector (2408). Angle (A3) may be, for example, from about 10° to about 90° (e.g.,)45°. Minor mount (2409) may be adjustably attached to base plate (2401) using one or more set screws (2414) and/or any other suitable attachment mechanisms. The distance between the mirror mount and the base plate, as well as the tilt angle of the minor, may be adjusted using set screws (2414). In certain variations, excitation module (134) may comprise one or more springs (2430) disposed between minor mount (2409) and base plate (2401). Springs (2403) may pull minor mount (2409) and base plate (2401) towards each other, or may push the mirror mount and base plate apart. Mirror (2406) may be attached to minor mount (2409) using, for example, one or more adhesives, such as a UV-curable optical adhesive (e.g., SK-9 or its equivalent).

Dichroic reflector (2408) may be selected to transmit laser beams from first laser (2402), and to reflect laser beams from second laser (2404). As shown, dichroic filter (2408) may be attached onto a reflector mount (2411) that may be adjustably attached to base plate (2401). The reflective surface of dichroic filter (2408) may be positioned in front of second laser (2404), such that the laser beam from the second laser is directed at an angle (A4) (FIG. 24B) towards cylindrical lens (2410). Angle (A4) may be, for example, from about 10° to about 90° (e.g.,) 45°. The laser beam from first laser (2402) may be transmitted straight through dichroic reflector (2408), and combined with the beam from second laser (2404) towards cylindrical lens (2410). In some variations, a dichroic reflector may reflect a portion of the laser beam from the first laser and transmit a portion of the laser beam from the second laser. For example, the laser beams from first and second lasers (2402) and (2404) may be directed towards a light sensor board (2418).

Light sensor board (2418) may monitor the power levels of the laser light, and provide an indication to a practitioner or computer control system to adjust the output power and/or pulse widths of the first and second lasers as needed. Light sensor board (2418) may comprise a photodiode (2420), a sensor lens (2422) configured to focus light onto the photodiode, and a connecter interface (2424). While light sensor board (2418) comprises a photodiode, other variations of light sensor boards may use different light detection devices. Light detection devices may be selected according to the spectral characteristics and intensity of the light they may capture. For example, a photodiode may be appropriate for light detection at certain light levels, while luminometers or photomultiplier tubes may be appropriate for light detections at other light levels. The amplification and sensitivity (e.g., gain), of photodiode (2420) may be adjusted according to spectral qualities of the excitation module laser beams.

In the configuration shown in FIGS. 24A and 24B, the laser beams from first and second lasers (2402) and (2404) are directed through a sensor lens (2422) and focused onto a photodiode (2420) of light sensor board (2418). In some variations, the position of light sensor board (2418) may be adjusted to align with the location of the laser beams, while in other variations, the light sensor board's position may be fixed. For example, light sensor boards comprising photodiodes that are large relative to the laser beam width may not require additional positional adjustments. Photodiode (2420) may detect the power levels of the laser beams from first laser (2402) and second laser (2404), and through feedback circuitry via connector interface (2424), electronically regulate the current through the laser diodes of the first and second lasers. In some variations, the power levels detected by photodiode (2420) may digitally converted (e.g., using a 24-bit analog-to-digital converter which may convert voltage output from the photodiode to digital signals) and used by a computer control system to normalize the laser pulse widths applied by the lasers. Electronic and/or computer control of the laser power output may help to prevent over- or under-exposure of the fluorescent markers.

As described above, laser beams may be frequency or amplitude modulated. For example, a first laser beam from a first laser may be modulated with a first carrier frequency, and a second laser beam from a second laser may be modulate with a second carrier frequency that is different from the first carrier frequency. The first and second laser beams may be simultaneously directed to the photodiode of a light sensor board. The light sensor board may have circuit logic capable of demodulating the frequency or amplitude modulated signals from the photodiode to extract the laser power data for each of the two lasers. In other variations, the light sensor board may transmit the modulated signals to a second board (e.g., a mainframe board), or to a computing device (e.g., an embedded PC), for demodulation. A variety of demodulation techniques may be implemented on a light sensor board, mainframe board, embedded PC, etc. For example, a light sensor board may demodulate signals using Fast Fourier Transform (FFT) or synchronous demodulation methods. Any known demodulation method may be implemented on a light sensor board, in accordance with the frequency or amplitude modulation of the laser signals to improve the signal-to-noise ratio and cross-talk rejection. As described below, frequency modulation of the laser beams that excite the fluorescent markers and demodulation of the emission wavelengths from the fluorescent markers may allow the cross-talk between emission data to be greatly reduced.

The laser beams from first and second lasers (2402) and (2404) may be combined and transmitted to cylindrical lens (2410), which may be mounted in a lens base (2413) and secured by set screws. Cylindrical lens (2410) may have an anti-reflective coating. Lens base (2413) may be adjustably attached to a housing of excitation module (134). Cylindrical lens (2410) may be adjusted via rotation around its optical axis (i.e., an imaginary line through the center of the lens), and/or translation along its optical axis. During use, the position and/or angles of the minor, dichroic reflector, and/or cylindrical lens may be adjusted so that the laser beams from both the first and second lasers are focused at the same plane (e.g., the plane may be the surface of the sample strip). The lasers, mirror, dichroic reflector, and cylindrical lens may be adjusted to attain a certain laser beam width at the surface of the sample strip. For example, the laser beam width may be less than or equal to 0.1 mm at the 1/ê2 power level, and the difference in the position of the beams from the first and second lasers may be less than 0.1 mm. In some variations, the geometry and optical characteristics of the cylindrical lens may vary according to the geometry of the test strip. For example, a cylindrical lens as shown in FIGS. 24A and 24B may be suitable for focusing laser beams onto striped or rectangular test strip bands. Alternatively, a different lens may be suitable for focusing laser beams onto circular, rounded test strip dots. For example, a double convex or planar convex lens with a focal distance of about 50 mm to 100 mm may be used to focus laser beams onto circular test strip dots. Other focal distances may be selected depending on the mechanical design of the excitation module. As the laser beams are collimated, the distance between the lens and the target may be approximately equal to the focal length of the lens. It may be advantageous to provide adjustability of the lens position in the direction of light propagation. This may help to compensate for possible imperfection of the lens and variability of its focal length. An objective lens may be used instead of a simple plano-convex or double-convex lens, to provide better focusing and compensate for focal length difference for two wavelengths used in the instrument.

As shown in FIGS. 24A and 24B, some variations of excitation module (134) may also comprise an aperture plate (2416) located underneath cylindrical lens (2410). Aperture plate (2416) may help reduce light scatter by a cartridge body containing a test strip. While aperture plate (2416) is depicted as an individual component, in some variations, an aperture plate may be integral with a housing of an excitation module. Aperture plate (2416) comprises an aperture (2417) (FIG. 24A) that is sized to permit passage of laser beams transmitted through the cylindrical lens, but to block any diffuse or scattered light. For example, the width of the laser beam that passes through the cylindrical lens may be from about 50 μm to about 150 μm (e.g., 100 μm). Accordingly, the diameter of aperture (2417) may be from about 70 μm to about 200 μm (e.g., 150 μm). In some variations, a filter may be provided over aperture (2417) to regulate the spectral characteristics of the light that falls on a test strip. Examples of filters that may be used in aperture plate (2416), and/or anywhere along the laser beam path described above, include neutral density filters, bandpass filters, longpass filters, dichroic reflectors, etc. Alternatively or additionally, an optically neutral glass plate may be provided over aperture (2417) to reduce any dust or debris from entering excitation module (134).

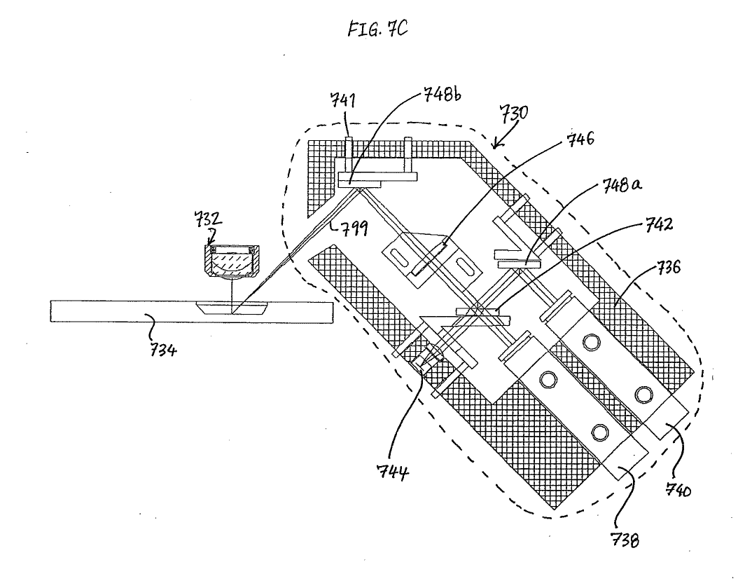

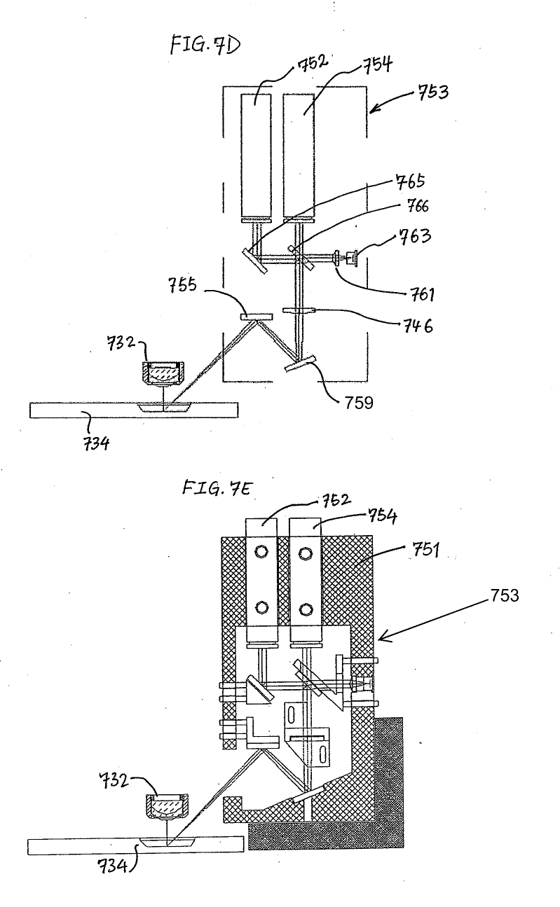

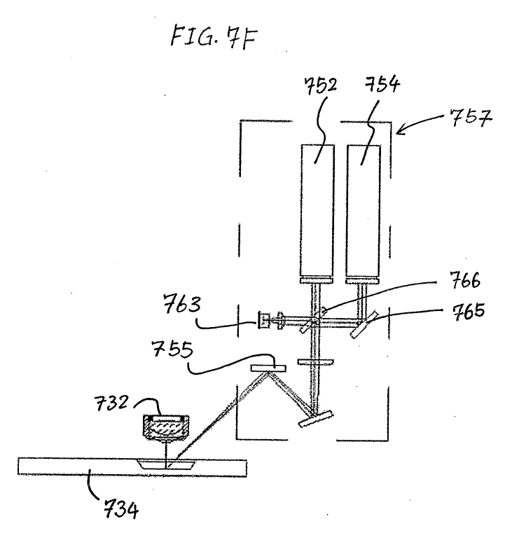

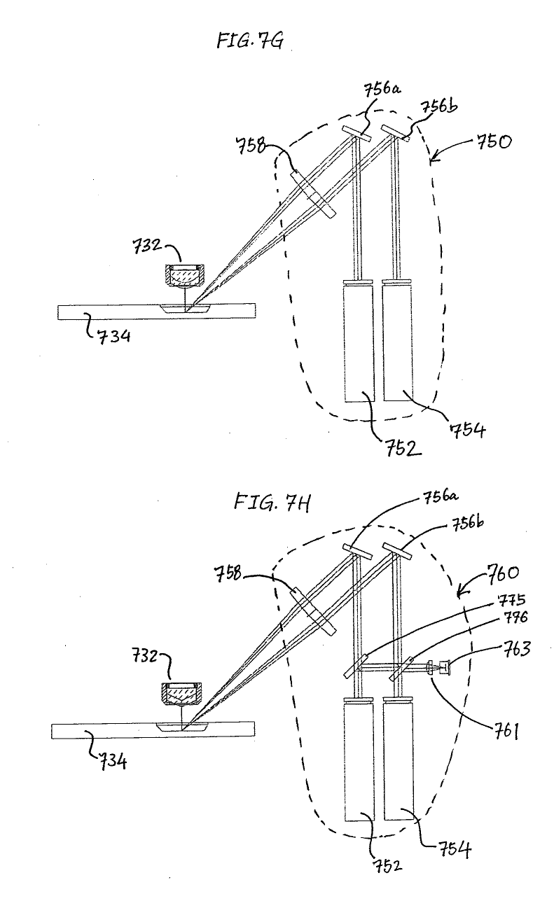

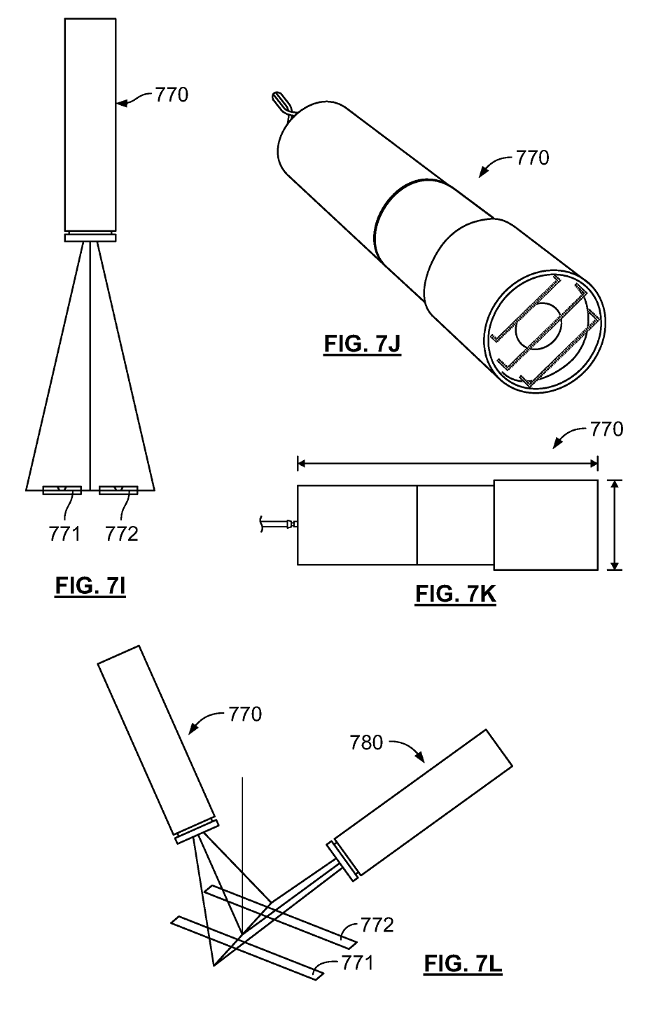

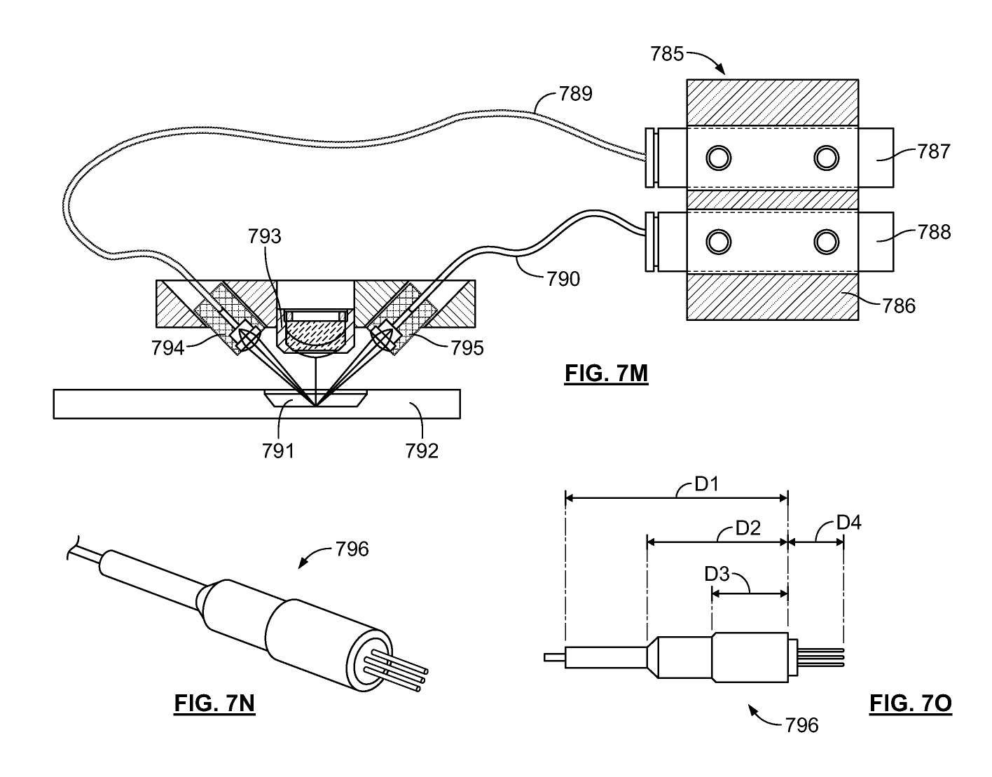

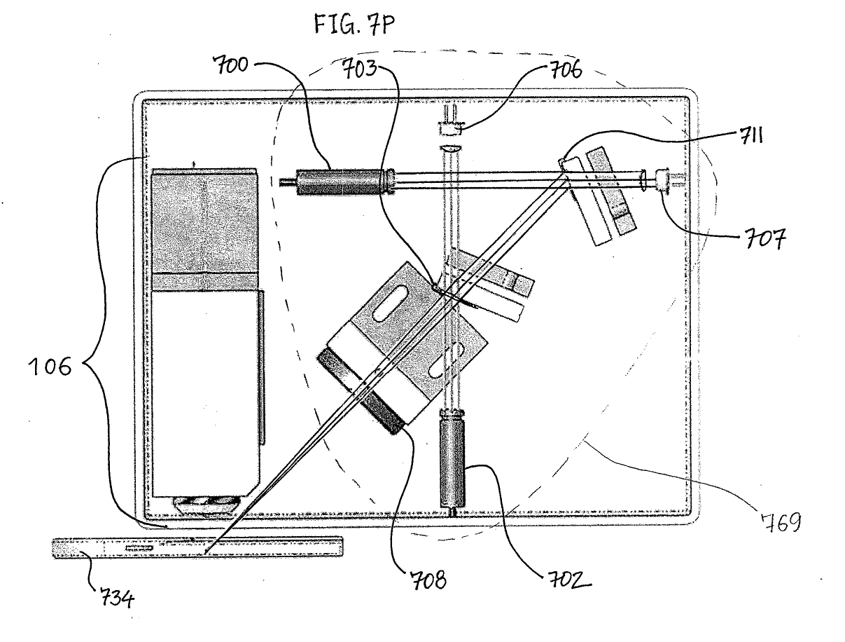

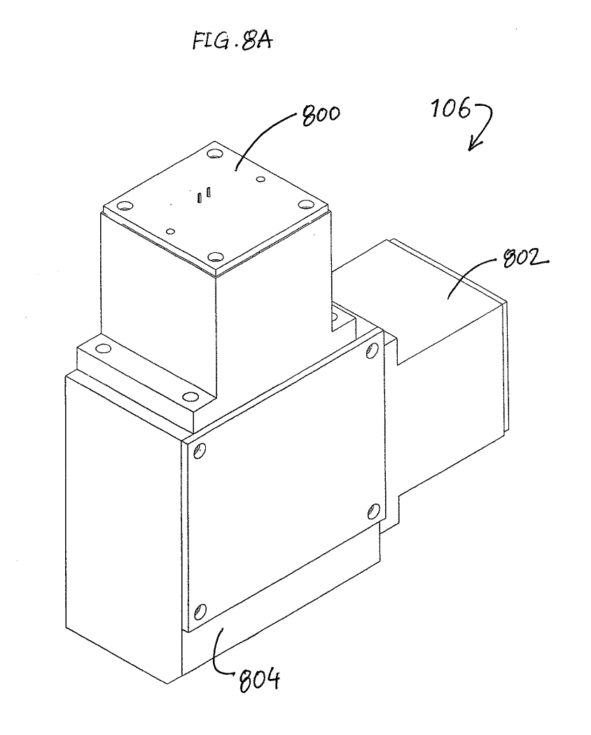

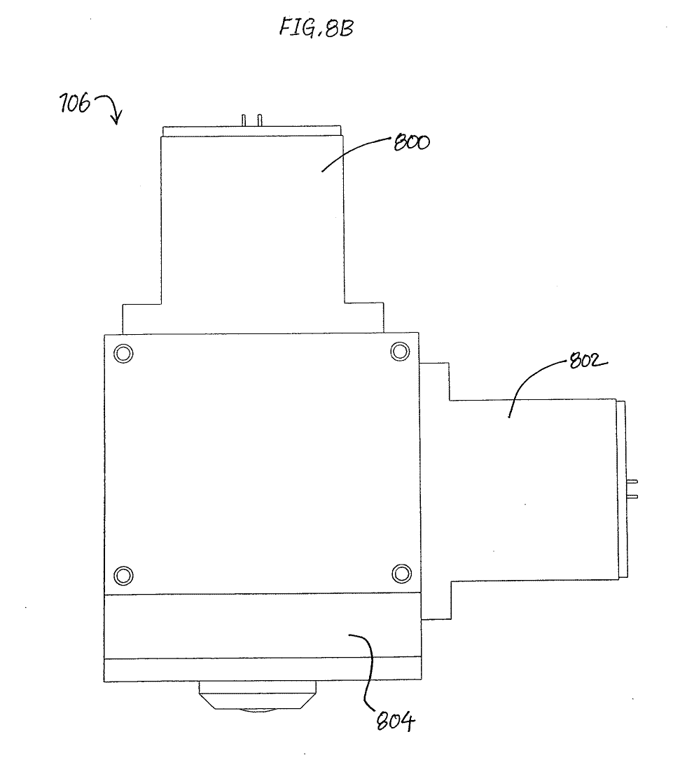

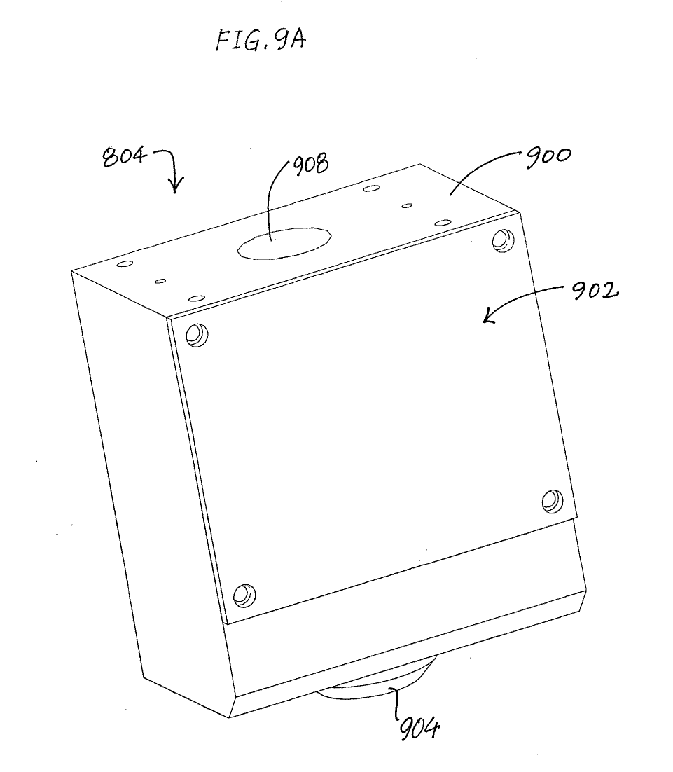

Other variations of excitation modules may be used in POC diagnostic systems for qualitative and/or quantitative analysis of one or more target analytes in a fluid sample. For example, FIGS. 7A and 7B show excitation module (104) from FIG. 1B in enlarged detail. Excitation module (104) comprises lasers (700) and (702) (which may emit laser beams of different wavelengths and intensities), a dichroic reflector (704), a photodiode (706), and a cylindrical lens (708). These components may be secured in position with respect to each other using, for example, an assembly of screws and mounts. In this variation, laser (700) is positioned by a laser mount (701), dichroic reflector (704) is positioned by a mirror mount (705), and cylindrical lens (708) is positioned by a lens housing (709). Mounts (701) and (705), and housing (709) may be adjustable, so that the relative positioning between laser (700), dichroic reflector (704) and cylindrical lens (708) may be altered. For example, the mounts and housing may be adjusted such that the laser beams emitted by lasers (700) and (702) are parallel to each other when they are directed to cylindrical lens (708) parallel to each other, which may allow them to be focused in the same location on the surface of a test strip. Alternatively, the mounts and/or housing may be in a fixed position, or a combination of fixed and movable mounts and/or housings may be used. The positions of mounts and housings may be adjusted manually (e.g., using screws that are externally accessible) and/or electromechanically (e.g., according to commands from a computer).

While excitation module (104) comprises two lasers (700) and (702), other variations of excitation modules may comprise one or more than two lasers. Lasers (700) and (702) may be any type of laser, such as a diode, solid state, gas, chemical, or metal-vapor lasers. In some variations, diode lasers may be used because of their compact size and ease of operation (e.g., the output power and/or the power modulation of a diode laser may be electronically and/or computer controlled). The operational wavelength of lasers (700) and (702) may be selected to match the excitation spectra of the fluorophores that are used. For example, the center frequency of lasers (700) and (702) may be chosen to match the excitation band for HiLyte Fluor™ 647 fluorophore and DyLite-800 fluorophore. Preferably, the laser wavelength should be matched with the wavelength that is maximally absorbed by the fluorophore. For example, laser (700) may emit at a wavelength of 635 nm, and laser (702) may emit at a wavelength between 750 to 800 nm. Alternatively, lasers (700) and (702) may be substituted with other light sources that provide sufficient excitation to the fluorophores of interest. Alternative excitatory light sources may include light-emitting diodes (LEDs), flash tubes, or any monochromatic lamps that can provide a sufficient intensity of light to induce emissions from the target fluorophore(s). The use of these light sources may require modifications to the optics of the excitation module, such as the inclusion of additional components (mirrors, filters, reflectors, condensers, etc).

While excitation module (104) employs dichroic reflector (704), other variations of excitation modules may use other optical components to achieve fundamentally the same effect. The system may include additional mirrors to direct laser beams to a photodiode (such as photodiode (706)), as well as to a cylindrical lens (such as cylindrical lens (708)). Other variations of excitation modules may employ other types of lenses, such as sphero-cylindrical lenses. This type of lens focuses the laser beam into a narrow line with a width of approximately 0.1-0.2 mm, which is defined by the combined optical power of the cylindrical and spherical components of the lens and by the properties of the initial laser beam. The length of this laser line is defined by the optical power of the spherical component of the lens. It may be adjusted by a proper lens selection to achieve the required configuration of the laser beam on the surface of substrate without reducing the laser power. Similar results may be achieved by using apertures which also allow laser beam shaping, although this approach may be associated with laser light losses. Alternatively, a spherical lens (plano-convex, bi-convex) may be used if the desired shape of the laser spot is circular (e.g., if the capture agents are coated onto the test strip as dots instead of bands). If a very sharp laser line is required (in the case of narrow test strip bands), then a high-quality objective lens or aspheric lens may be used. If the wavelengths of the lasers differ significantly, it may be advantageous to use achromatic optics, which reduces the wavelength dependence on focusing. In some variations, the raw laser beam may provide sufficient fluorophore excitation without the use of any lenses.

During use of an excitation module, such as excitation modules (104) or (134), a variety of laser pulse sequences may be applied to one or more test strips to excite the fluorophore or fluorophores of interest. Individual laser pulses may vary in intensity (e.g., power) and pulse width, while a sequence of pulses may vary in periodicity and duty cycle. For non-periodic laser pulses, the inter-pulse interval may also vary. These are examples of pulse sequence parameters that may be adjusted to elicit the strongest fluorescent signal from a fluorophore, and to reduce photobleaching. Laser pulses provided by two lasers, where each laser applies beams of different wavelengths, may be interleaved temporally, such that no single spot on a test strip is illuminated by both wavelengths of laser light. Each laser may also apply laser pulse sequences with different characteristics (e.g., different periodicities, duty cycles, etc.), which may simplify emission detection and allow for cross-talk correction. In some variations, the excitation of both lasers may be applied simultaneously or with a short interval therebetween. For example, pulse widths may vary from about 10 microseconds to about 1 millisecond.

In some variations, laser pulses may be frequency or amplitude modulated to reduce cross-talk between lasers emitting different wavelengths of light. Modulation of laser pulses may also help to reject noise from any stray light. For example, a first laser emitting light of a first wavelength may be frequency modulated with a 3 kilohertz (kHz) carrier signal, and a second laser emitting light of a second wavelength may be frequency modulated with a 6 kHz carrier signal. Without being bound by theory, it is believed that frequency modulation of a first laser beam with a carrier frequency of N and frequency modulation of a second laser beam with a carrier frequency of 2N provide theoretically perfect cross-talk rejection when using synchronous demodulation methods. The frequency or amplitude modulation of the laser pulses may be controlled by an electric circuit, or may be controlled by a computing device. A computing device (e.g., circuitry on a light sensor board and/or an embedded PC), may demodulate the emission data of a tag or marker as previously described (e.g., using FFT or synchronous demodulation methods). Frequency modulation of the laser beams from two different lasers using two different carrier signals may be desirable when the laser beams excite two different fluorescent tags at the same location on a test strip, since demodulating the emission wavelengths of the different fluorescent tags allows them to be independently analyzed and evaluated. As described previously, light sensor boards may have demodulation circuitry to remove the carrier frequency to extract the signal that arises from each of the different fluorescent tags.

Of course, other variations of excitation modules, such as excitation modules having similar components that are arranged differently, may be used. For example, FIG. 7C shows an excitation module (730) having a different configuration from previously shown excitation modules, and comprising additional components. FIG. 7C also shows an objective lens (732) and a cartridge (734) as a frame of reference. As shown in FIG. 7C, excitation module (730) comprises a housing (736), two lasers (738) and (740), a dichroic reflector (742), a photodiode (744), a cylindrical lens (746), and minors (748a) and (748b). This arrangement of components may, for example, provide a different light path from the variation shown in FIGS. 7A and 7B. The type of arrangement that is used for a given optical module may depend, for example, on space constraints that dictate the dimensions of the optical module housing. In some variations, housing (736) may allow for enhanced accessibility to internal excitation module components (e.g., for adjustment). For example, alignment screws (741) may be externally accessible and may be adjusted to adjust the direction of laser beam (799).

FIGS. 7D and 7E show another variation of an excitation module (753) having a different configuration (once again, with objective lens (732) and cartridge (734) as a frame of reference). Excitation module (753) comprises lasers (752) and (754) that are adjacent to each other, and a photodiode (763) (FIG. 7D) that is oriented perpendicularly to the laser beam paths, closest to laser (754). The beams are directed to a photodiode (763) by a mirror (765) through a photodiode lens (761) and a dichroic filter (766) (FIG. 7D). Dichroic filter (766) also directs the beams to cylindrical lens (746), which then directs the beams toward a series of minors (759) and (755), to cartridge (734) (FIG. 7D). In some variations, these optical components may be retained and positioned in a housing, such as housing (751) shown in FIG. 7E. In other variations, the components may not be enclosed in a housing, but may be secured and positioned using an assembly of clamps and beams, for example.

Alternate arrangements of functionally analogous components may also be used. For example, FIG. 7F shows an arrangement of an excitation module (757) in which photodiode (763) is positioned closer to laser (752). While the components of excitation module (757) are arranged differently from the components of excitation module (753), both excitation modules may achieve essentially the same effect in terms of laser beam delivery. Other configurations may be used that have any appropriate number of minors, and/or that have a shorter or longer light path, for example.

FIG. 7G shows another variation of an excitation module (750), with objective lens (732) and cartridge (734) as a frame of reference. This arrangement utilizes fewer optical components than other variations (e.g., fewer minors, filters, reflectors, and photodiodes), and as such may occupy less space. Excitation module (750) comprises lasers (752) and (754) (which may emit laser beams of different wavelengths and intensities), minors (756a) and (756b), and a cylindrical lens (758). Minors (756a) and (756b) may be adjustable to allow for adjustment of the laser beams so that they propagate parallel to each other before falling onto the surface of cylindrical lens (758). This allows focusing of both beams at the same location of the test strip.

FIG. 7H depicts an additional variation of an excitation module (760), which includes components that are not present in excitation module (750). The additional components include glass plates (775) and (776), photodiode (763), and photodiode lens (761). Glass plates (775) and (776) may be thin glass plates, which reflect a small portion (e.g., about 8%) of the incident light while allowing most of the incident light to pass through. The reflected light may be directed through photodiode lens (761), towards photodiode (763). Photodiode lens (761) may be fixedly or adjustably positioned. While excitation module (760) comprises more components than excitation module (750), the additional photodiode may provide for laser power sensing, which may allow for more precise control of lasers (752) and (754) by normalizing every laser pulse. In some variations, an excitation module may comprise glass plates with an anti-reflective coating to regulate the amount of laser power directed to the photodiode (e.g., so that the amount of laser power directed to the photodiode is not excessively high).

FIG. 7P illustrates an additional variation of an excitation module (769), with detection module (106) and cartridge (734) as a frame of reference. Excitation module (769) comprises lasers (700) and (702), photodiodes (706) and (707), a dielectric mirror (711), a dichroic filter (703), and cylindrical lens (708). Lasers (700) and (702) may be arranged such that their laser beams are orthogonal to each other. Photodiodes (706) and (707) may each detect the laser beam from one of lasers (702) and (700) respectively, as compared to other variations where a single photodiode detects the laser beams from both lasers. This may allow for tailored, individual control of each of lasers (700) and (702). Dielectric mirror (711) may be used to selectively reflect and/or transmit the laser beam from laser (700). The high wavelength specificity of a dielectric minor may be desired to reduce non-specific light transmission; however, other reflective and/or transmissive optical components may also be used, such as glass plates or filters. As previously described, alternate optical components may be used in excitation module (769) and may be arranged in any way to achieve a similar optical effect in terms of laser beam delivery to cartridge (734).

An additional variation of an excitation path is depicted in FIG. 7I. The path shown in FIG. 7I may be especially advantageous, for example, when light is being applied to relatively small cartridges. FIG. 7I shows the use of a laser diode module (770) with integrated line generating optics (shown in more detail in FIGS. 7J and 7K) to simultaneously excite two different cartridges (771) and (772). Laser diode module (770) may, for example, exhibit enhanced efficiency in assaying samples, since it may be used to assay multiple samples simultaneously. FIG. 7L depicts laser diode module (770) being used in conjunction with another laser diode module (780) to simultaneously excite two different cartridges (771) and (772). In some variations, laser diode module (770) may comprise a red laser. Alternatively or additionally, laser diode module (780) may comprise an infrared laser. In certain variations, the excitation paths depicted in FIGS. 7I and 7L may be relatively short, which may allow for a reduction in the overall size of the excitation module. In some variations, one or more other optical components may be included for additional beam shaping. Moreover, additional shielding may be included to limit or prevent cross-talk (e.g., unintended excitation and/or blurred emission readings) between cartridges (771) and (772).

Still other variations of excitation modules may be used. For example, in some variations, an excitation module may comprise one or more fiber-coupled lasers. As an example, FIG. 7M shows an excitation module (785) comprising a laser holder (786), lasers (787) and (788) (which may apply laser beams of different wavelengths and intensities) disposed in laser holder (786), and optical fibers (789) and (790) connected to lasers (787) and (788), respectively. Optical fibers (789) and (790), each of which may be a single fiber or fiber bundles, transmit light from lasers (787) and (788) and onto a test strip (791) disposed within a cartridge (792). In some variations, excitation module (785) may further comprise focusing modules (794) and (795), which may compensate and correct for any laser dispersion that may occur during beam transmission through optical fibers (789) and (790).

The use of fiber-coupled lasers, such as lasers (787) and (788), may allow for the excitation module to be relatively small. Fiber-coupled lasers (787) and (788) may emit laser light of different wavelengths and intensities, for example, 635 nm light at about 0.5 mW to about 20 mW (e.g., 8 mW), and/or 785 nm light at about 0.5 mW to about 30 mW (e.g., 20 mW), or any other range of wavelengths and power intensities. For example, one laser may emit at an intensity of about 5 mW (e.g., for detecting the control analyte), while a second laser may emit at an intensity of about 40 mW (e.g., for detecting the test analyte). In some variations, for example, a battery-operated diagnostic system having relatively low power consumption may be achieved by using lasers that emit at no more than 5 mW. In some cases in which an excitation module includes fiber-coupled lasers (e.g., laser (796) shown in FIGS. 7N and 7O), it may not be necessary for the excitation module to include other optical components, such as minors, filters, reflectors, photodiodes, or lenses. As a result, the space occupied by the excitation module (and the optical module) may be reduced. Additionally, the control of the excitation module may be simplified.

As shown in FIG. 7O, laser (796) has a first dimension (D1) that may be about 33.61 mm, a second dimension (D2) that may be about 21.26 mm, a third dimension (D3) that may be about 11.61 mm, and a fourth dimension (D4) that may be about 8 mm, for example. These dimensions may vary depending on the laser model and the manufacturer. While not discussed in further detail here, FIG. 7M also shows an objective lens unit (793) of a detection module (the rest of which is not shown).

### Detection Module

Various types of detection modules may be used in a POC diagnostic system for qualitatively and/or quantitatively assaying a fluid sample to detect one or more analytes in the fluid sample. The detection mechanism of a detection module may vary according to the types of tags or markers that bind the target analyte. For example, a detection module with magnetic sensors may be used to detect target analytes tagged with magnetic-based markers. As described above, target analytes may be tagged with fluorescent markers, and a detection module may have one or more light-based sensors that may be used to capture emission wavelengths. Some variations of detection modules may comprise one or more detector units that are each configured to detect fluorescent emissions of one fluorescent marker, which typically emits in a spectral band 10 nm to 50 nm wide. However other variations of detector units may be configured to detect fluorescent emissions in a narrower or wider spectral range, or may detect emissions of one or more spectral bands. Moreover, in certain variations, a detection module may comprise more than two detector units (e.g., in the event that more than two different fluorophores are being used to detect analytes in a sample). Some variations of detector units may be configured to detect multiple wavelengths of emitted fluorescent signals. In such variations, a single detector unit may be used to detect fluorescence from multiple different fluorophores. Any number of detector units may be included in the optical module as needed to detect the fluorescent signals of interest. In some variations, the detector units may be positioned orthogonally with respect to each other; however, in other variations, the detector units may be positioned differently relative to each other (e.g., substantially parallel, or at a non-orthogonal angle). The positioning of the detector units in a detection module may depend, for example, on the alignment and positioning of the tray and sample cartridge relative to the detection module, and/or on the alignment and positioning of the excitation module relative to the detection module.

A detection module may also comprise one or more optical elements that may help to focus and direct light to the appropriate detector unit. In some variations, the optical element may direct multi-spectral light to different detector units. For example, a detection module may comprise an objective lens which may, for example, gather the fluorescent emissions from a test sample and focus the fluorescent emissions, such that the resulting signal can be detected by the detector units. A detection module may also comprise one or more dichroic filters or reflectors to direct the light path of different fluorescent emissions to different detector units. Suitable dichroic filters include those that are capable of reflecting light emitted by a first fluorophore in the test sample (e.g., a first fluorophore that is conjugated to an analyte-binding agent), and transmitting light of a different wavelength that is emitted by a second fluorophore in the test sample (e.g., a second fluorophore that is conjugated to a control analyte). Other variations of objective lens units may alternatively or additionally comprise other optical components that may achieve fundamentally the same optical effect, such as mirrors, any type of suitable filter (e.g., neutral density filters, notch filters, interference filters, etc.), and/or dichroic reflectors.

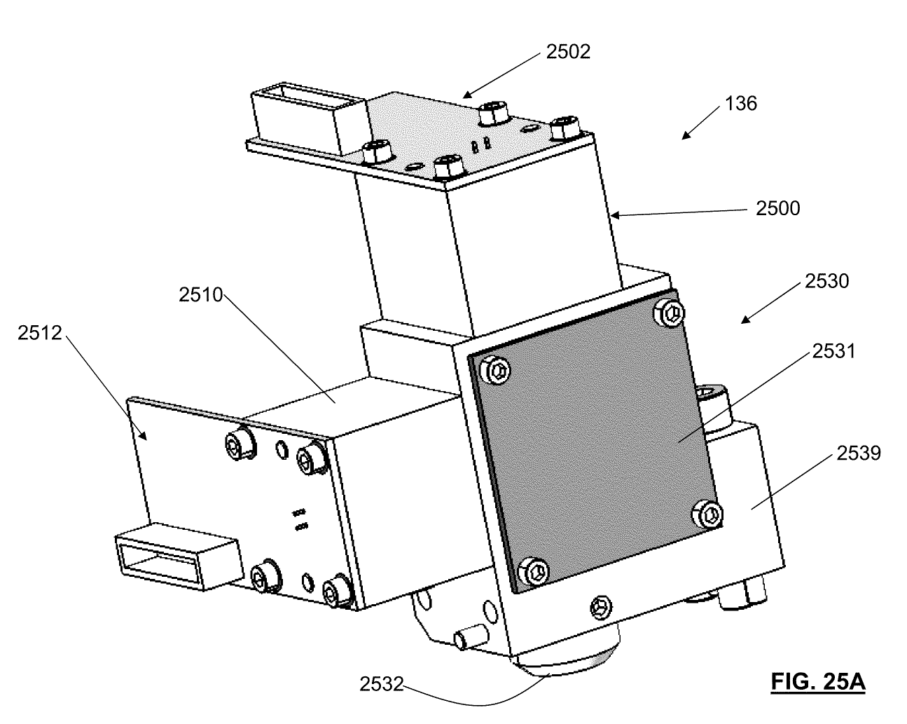

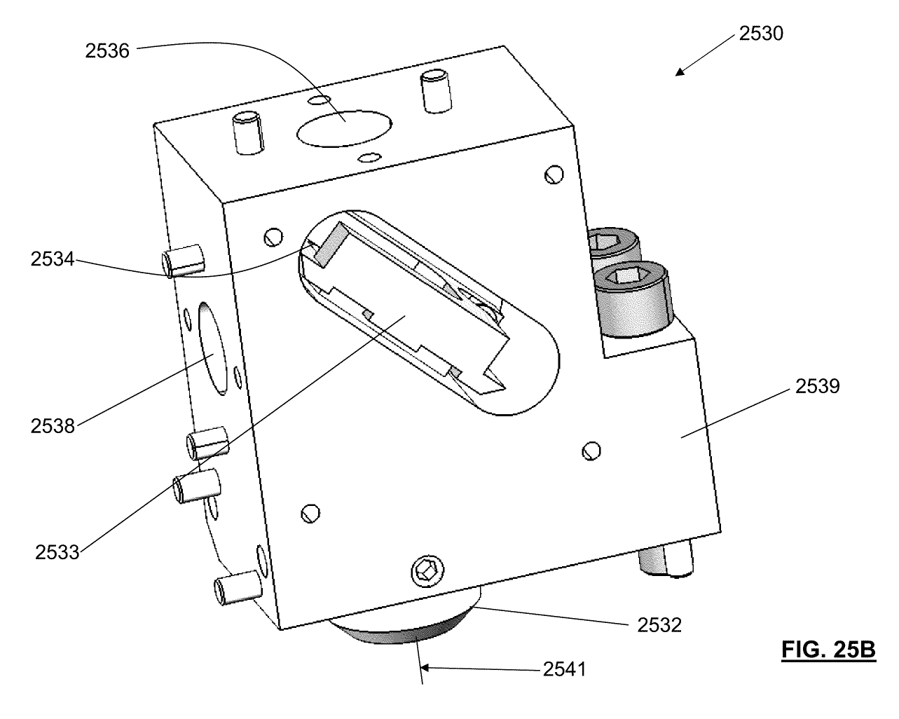

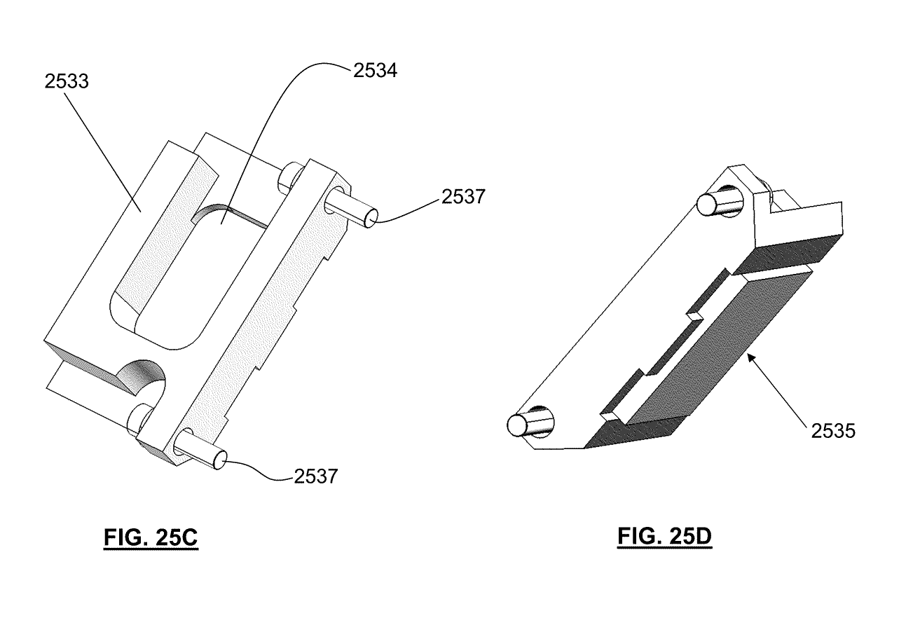

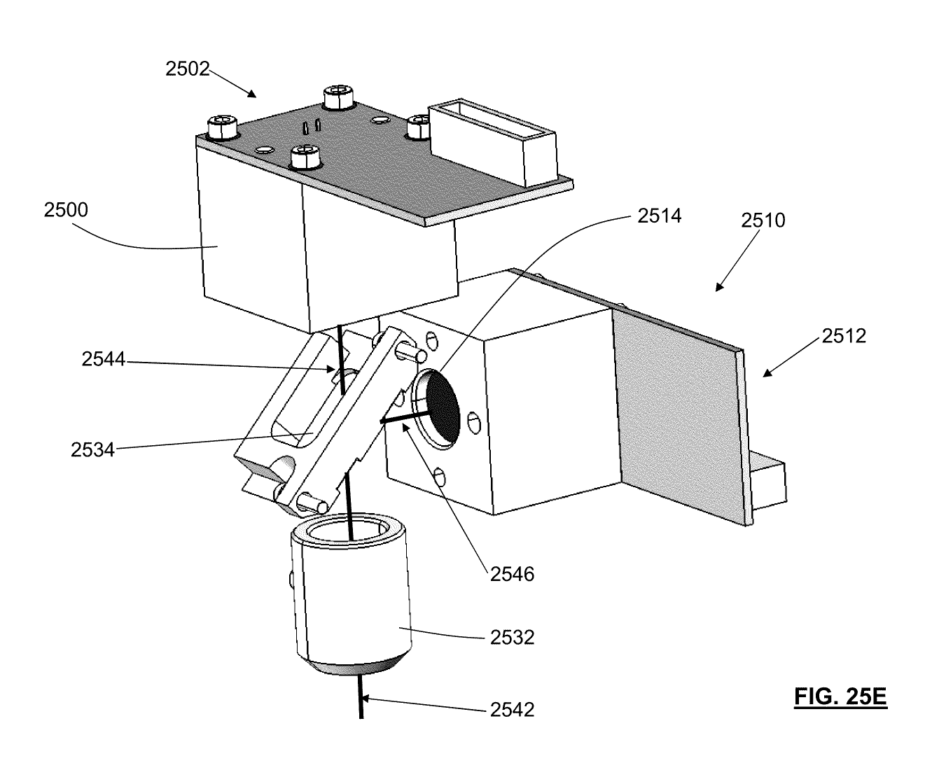

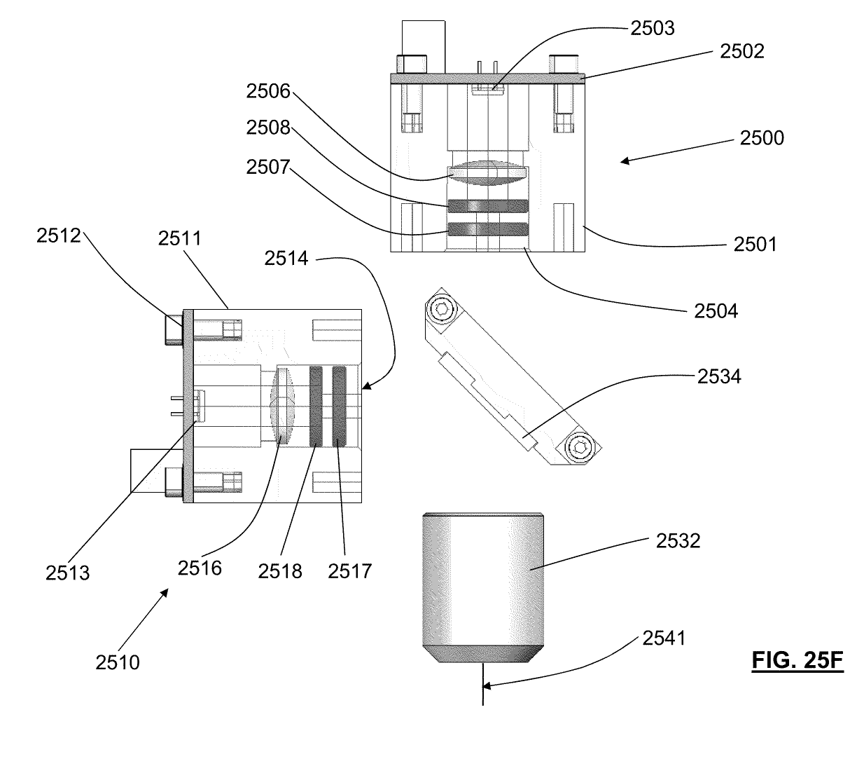

Examples of detection modules that may be used in a diagnostic detection system are described below. One example of a detection module is detection module (136) of FIG. 1A, which is shown in enlarged detail in FIGS. 25A-25F. As shown there, detection module (136) comprises an objective lens unit (2530), a first detector unit (2500) attached to a first surface of the objective lens unit, and a second detector unit (2510) attached to a second surface of the objective lens unit that is perpendicular to the first surface. Detection module (136) may also comprise an opaque cover (2531) that is attached on one side of objective lens unit (2530), which may reduce light scattering and interference (which may cause the light signal-to-noise ratio to increase). Additionally, opaque cover (2531) may help prevent eye exposure to harmful fluorescent emissions. First and second detector units (2500) and (2510) may each comprise a light sensor board (2502) and (2512), respectively. In some variations, first detector unit (2500) may be configured to analyze light with a first emission spectrum, and second detector unit (2510) may be configured to analyze light with a second emission spectrum.

FIG. 25B depicts a perspective view of objective lens unit (2530), with opaque cover (2531) removed. As shown there, objective lens unit (2530) comprises a housing (2539), a dichroic filter (2534), and an objective lens (2532) that is arranged to collect light to the dichroic filter. Housing (2539) comprises a first aperture (2536) in a top surface, and a second aperture (2538) in a side surface that is perpendicular to the top surface. Additionally, housing (2539) comprises an aperture (not shown) that is sized and shaped for objective lens (2532). Objective lens (2532) may be adjustably or fixedly attached to housing (2539). For example, the objective lens may be attached by screw-fit, snap-fit, adhesion using SK-9, etc. The objective lens may be adjusted and positioned such that the emission light from the fluorescent markers may be directed to dichroic filter (2534). Objective lens (2532) may also have an anti-reflective coating to prevent light scattering, and may be any lens type suitable for focusing emission wavelengths from fluorescent markers (e.g., an achromatic objective lens or an aspheric lens). A singlet lens may be used as well, according to the desired image quality. It may be advantageous to use a lens with an antireflective coating to increase sensitivity and reduce potential background levels.

Dichroic filter (2534) may be selected according to the emission spectra of the fluorescent markers of interest. Dichroic filter (2534) may transmit light with a first emission spectrum through first aperture (2536), and reflect light with a second emission spectrum through second aperture (2538). As will be described later, light transmitted through first aperture (2536) may be captured and analyzed with first detector unit (2500), and light reflected through second aperture (2538) may be captured and analyzed with second detector unit (2510). For example, dichroic filter (2534) may transmit light with a wavelength of about 674 nm, while reflecting light with a wavelength of about 794 nm. In some variations, a commercially available interference dichroic filter may be used, while in other variations, a custom-built filter may be used (e.g., Omega Optical, Vermont, USA). Dichroic filter (2534) may be retained in a filter holder (2533) (FIG. 25B) such that a portion of the light transmitted from objective lens (2532) is directed through first aperture (2536), and a portion of the light is directed through second aperture (2538). Referring to FIGS. 25C and 25D, dichroic filter (2534) may be attached to filter holder (2533) by adhesion (e.g., using UV curable adhesive, SK-9, etc.) such that the reflective surface (2535) of dichroic filter (2534) is facing downward. Filter holder (2533) may be adjustably or fixedly attached to housing (2539) of objective lens unit (2530) using one or more screws (2537) (FIG. 25C). In some variations, filter holder (2533) may be attached or adjusted such that dichroic filter (2534) is at an angle with respect to optical axis (2541) of objective lens (2532). For example, dichroic filter (2534) may be attached such that it forms an angle with optical axis (2541) that may be from about 20° to about 80°. It should be noted that while a dichroic filter is described here, any optical components that can perform a similar optical function may be used, such as notch filters, bandpass interference filters, or any combination thereof, or any optically similar configurations.

FIG. 25E depicts detection module (136) without objective lens unit housing (2539). As shown there, first and second detector units (2500) and (2510) may each have an aperture that is sized and shaped to be aligned with first and second apertures (2536) and (2538) of objective lens unit (2530). For example, second detector unit (2510) may be attached and aligned to objective lens unit (2530) such that its second detector aperture (2514) is aligned with second aperture (2538). In this configuration, emission light (2542) (e.g., from the fluorescent markers on a test strip), may be gathered and focused through objective lens (2532) and directed to dichroic filter (2534). Dichroic filter (2534) may transmit light with a first emission spectrum (2544) to first detector unit (2500), and reflect light with a second emission spectrum (2546) to second detector unit. Light with a first emission spectrum (2544) may be collected and analyzed by first light sensor board (2502), separately from light with a second emission spectrum (2546), which may be collected and analyzed by second light sensor board (2510). For example, emission light (2542) from a test strip may have a spectrum from about 650 nm to about 800 nm. Dichroic filter (2534) may transmit light with emission wavelengths from about 625 nm to about 675 nm to first detector unit (2500), and reflect light with emission wavelengths from about 750 nm to about 800 nm to second detector unit (2510).

Detector units may comprise one or more optical components that may direct light of a targeted emission spectrum to a photosensing device on a light sensor board (e.g., a photodiode as previously described). Optionally, detector units may comprise one or more optical components that filter out light with emission spectra outside of the targeted emission spectrum to improve the signal-to-noise ratio. Referring now to FIG. 25F, first detector unit (2500) comprises a housing (2501) that retains a sensor lens (2506), and a first filter (2507) and a second filter (2508) that adjusts the spectral characteristics of incident light. As indicated previously, housing (2501) may comprise a first detector aperture (2504) configured to be aligned with first aperture (2536) of objective lens unit (2530). Second detector unit (2510) comprises a housing (2511) that retains a sensor lens (2516) and a first filter (2517). Optionally, second detector unit may comprise a second filter (2518). While the detector units described here are configured to accommodate one or two filters, in other variations, detector units may be configured to accommodate more than two filters. Filters may be secured in the detector unit housing by adhesives, friction-fit, twist-fit, etc. The filters, sensor lenses, and photodiodes of the light sensor boards may be adjusted and/or positioned such that the light directed to the photodiode is appropriately focused for accurate and precise detection. For example, the distance and tilt angle between the above elements may be adjusted by a practitioner, or may be adjusted and fixed during manufacturing.

Filters (2507), (2508), (2517), and (2518) may be any suitable optical component, for example, interference band pass filters, notch filters, glass filters, and the like, depending on the fluorescent marker emission spectrum of interest. For example, in some variations of detection module (136), dichroic filter (2534) may be selected to transmit red spectrum light to first detector unit (2500) and reflect infrared spectrum light to second detector unit (2510). The red spectrum light directed to first detector unit (2500) may be transmitted through a red band pass filter (2507), and a red glass filter (2508), and focused by sensor lens (2506) onto photodiode (2503) of first light sensor board (2502). The infrared spectrum light directed to second detector unit (2510) may be transmitted through an infrared interference band pass filter (2517) and focused by sensor lens (2516) onto photodiode (2513) of second light sensor board (2512). Optionally, infrared spectrum light may be additionally filtered by second filter (2518) (e.g., a glass filter) if desired. As described previously, the power levels detected by the photodiode may digitally converted (e.g., using a 24-bit analog-to-digital converter which may convert voltage output from the photodiode to digital signals) and/or demodulated, and transmitted to a mainframe board or computing device for further processing and analysis.

POC diagnostic system (100) from FIG. 1B comprises another variation of a detection module (106), which is depicted in enlarged detail in FIGS. 8A and 8B. As depicted there, detection module (106) comprises two detector units (800) and (802), as well as an objective lens unit (804).

Detector units (800) and (802) and objective lens unit (804) may be in the form of individual components that are coupled to each other. As shown, the detector units are positioned orthogonally relative to each other. Additionally, while each of the detector units and the objective lens unit is in a separate housing that is then attached (e.g., screwed, bolted, welded, etc.) to the other housings, in certain variations, at least some (e.g., all) of the various units of a detection module may be placed in a single housing. The single housing may, for example, have a similar shape to the overall shape of the individual housings when they are coupled to each other.

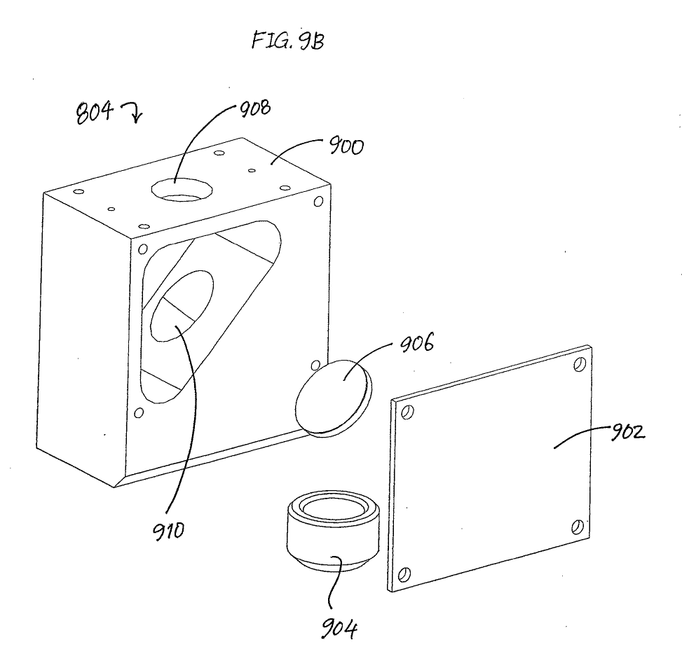

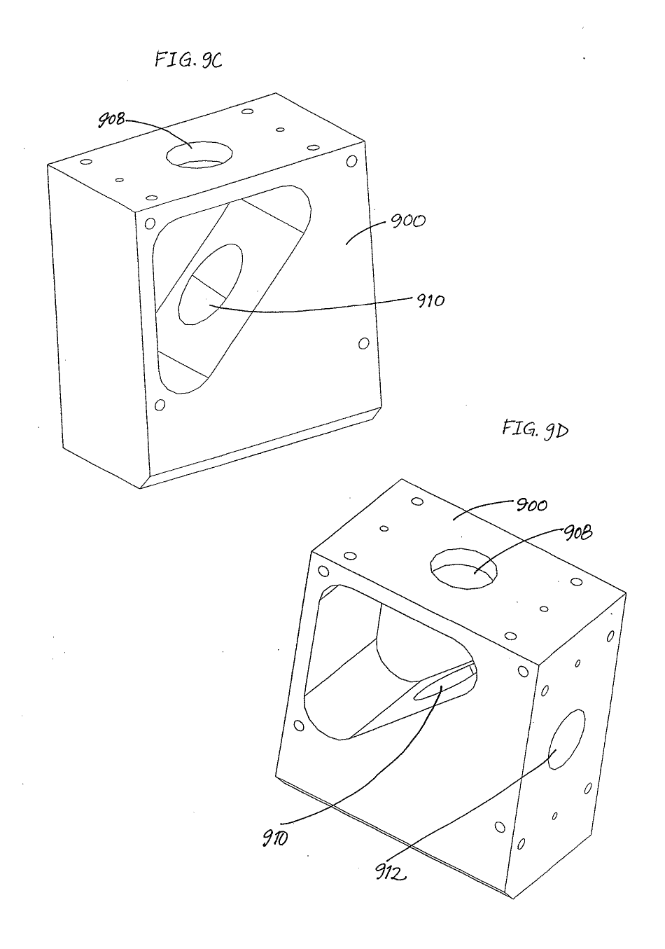

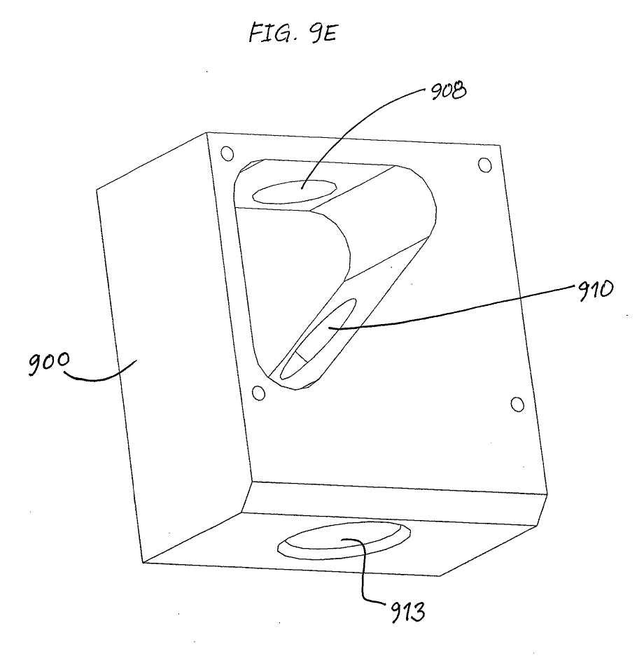

FIGS. 9A-9E show objective lens unit (804) and its various components in enlarged detail. As shown in FIGS. 9A and 9B, objective lens unit (804) comprises a housing (900) with a removable face (902), an objective lens (904), and a dichroic filter (906). Housing (900) includes apertures (908), (910), (912), and (913), as shown in FIGS. 9B-9D. Aperture (910) is shaped and positioned to accommodate dichroic filter (906). Apertures (908) and (912) are shaped and positioned such that light reflected or transmitted from dichroic filter (906) (when secured in aperture (910)) can pass through both apertures unimpeded. Detector units (800) and (802) may be positioned to detect light that passes through apertures (908) and (912), respectively. Finally, aperture (913) (FIG. 9E) is configured to secure objective lens (904), and to position objective lens (904) so that fluorescent emissions may be directed to dichroic filter (906).

Removable face (902) may, for example, reduce light scattering and interference (which may cause the light signal-to-noise ratio to increase). Additionally, removable face (902) may help prevent eye exposure to harmful fluorescent emissions. Removable face (902) may be made of any optically shielding material, which may be translucent or opaque. Removable face (902) may be made of the same material or materials as the rest of housing (900), or may be made of a different material or materials.

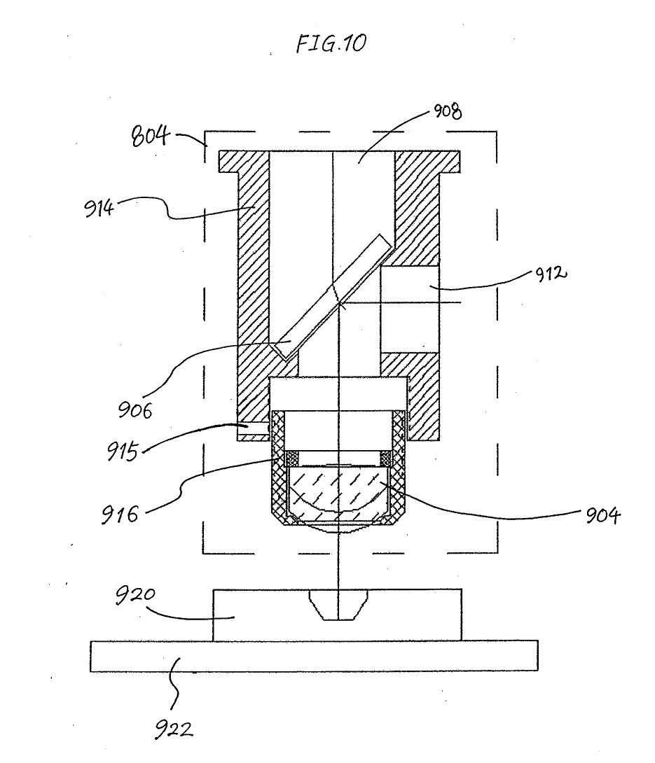

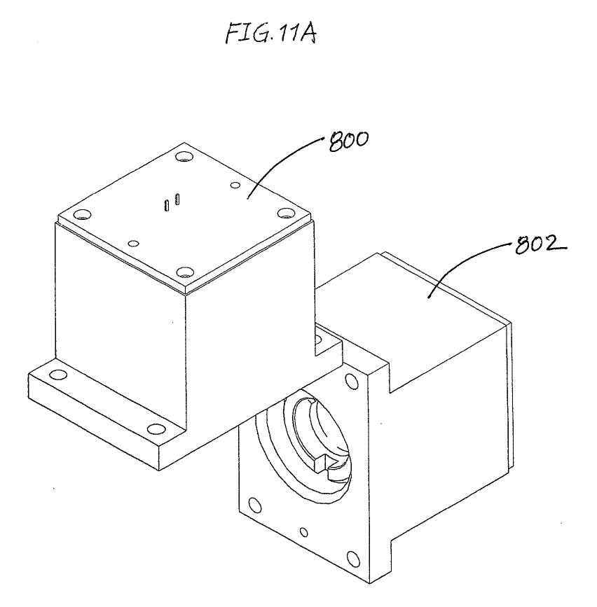

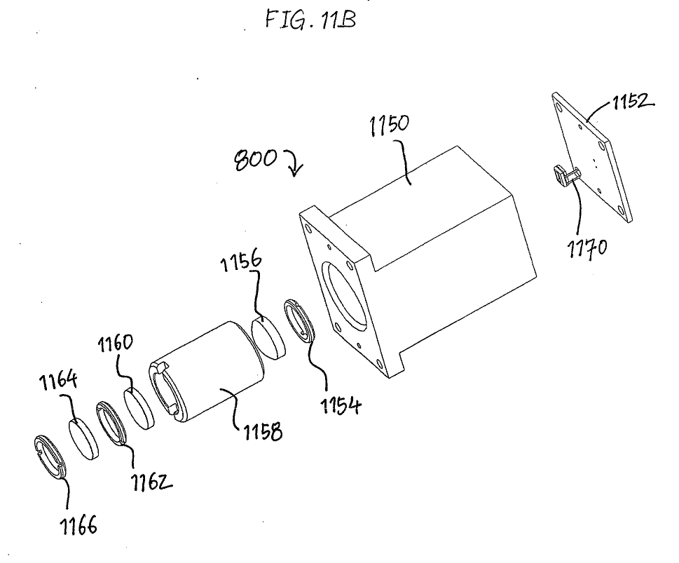

FIG. 10 shows a cross-sectional view of objective lens unit (804), when the objective lens unit is positioned over a cartridge (920). As shown there, objective lens unit (804) also comprises a baffle (914), a set screw (915), and an adjustable mount (916). Baffle (914) may help to reduce collection of scattered and stray light and may comprise light scattering reduction features, such as a threaded internal surface. In some variations, baffle (914) may be integrally coupled with housing (900). Adjustable mount (916) may allow for adjustment of the relative positions of the optical components, such as the distance between objective lens (904) and cartridge (920). A set screw (915) fixes the position of objective lens (904) after completion of alignment, in order to prevent possible misalignment due to vibrations or perturbations (e.g., during shipment). Set screws may also be provided in other locations of the objective lens unit to adjust and align other components in the unit.

As described previously, objective lens (904) is positioned to gather fluorescent emissions from the sample in cartridge (920), and to direct the gathered fluorescent emissions in a focused manner to the detector unit(s). Objective lens (904) may be any suitable type of lens that achieves adequate focusing, such as achromatic objective lens. Typically, objective lens (904) may be of a sufficient quality to produce a well-collimated beam, which may allow better utilization of filtering capabilities of interference band pass and dichroic filters. Depending on the required level of performance, in some variations, a less complex aspheric lens may be used. The contents of cartridge (920) may be scanned and analyzed by positioning objective lens unit (904) directly over cartridge (920), and moving optical module (101) relative to cartridge (920). This may be achieved, for example, by moving the optical module, the cartridge, or both. In some variations, cartridge (920) may be coupled to a motorized tray (922), the movement of which may be controlled by a computer. The function and control of motorized tray (922) will be discussed in more detail below.

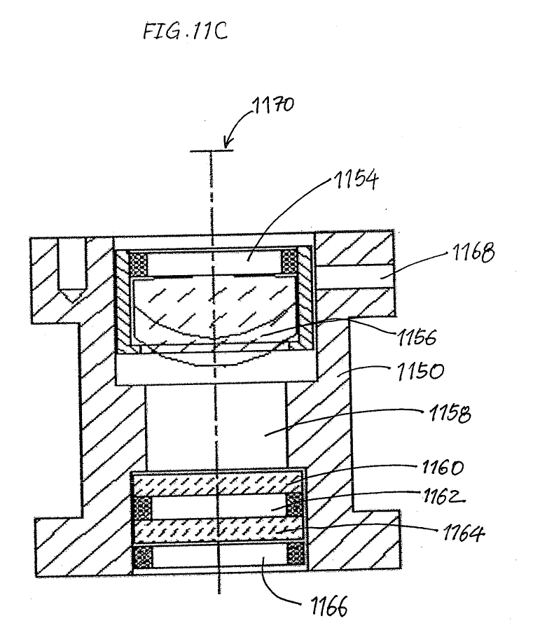

FIGS. 11A-11C depict detector units (800) and (802) of detection module (106) in enlarged detail.

First, FIG. 11A is an illustrative view depicting the positioning of detector units (800) and (802) relative to each other in detection module (106). While detector units (800) and (802) are positioned as shown, it should be understood that other variations of detection modules may comprise detector units that are positioned differently with respect to each other, or may comprise multiple detector units that are contained within a single housing. The positioning of a detector unit may be determined by space constraints, the interface with an objective lens unit, the number of detector units in the detection module, and/or any of a number of other different factors.

Detector unit (800) is shown in an exploded view in FIG. 11B, and in a cross-sectional view in FIG. 11C. Detector unit (802) may be essentially the same as detector unit (800) or quite similar, or the two detector units may be different from each other. In some variations, detector units (800) and (802) may each comprise different filters tailored to a different emission spectrum of a different fluorophore. This may, for example, allow the detector units to be used to detect the fluorescence of two fluorophores with different emission spectra. Of course, additional detector units may be added (e.g., to detect the fluorescence of additional fluorophores).

As shown in FIGS. 11B and 11C, detector unit (800) comprises a housing (1150), a photodiode (1170) and a cover mount (1152), a retaining ring (1154), a lens (1156), a lens holder (1158), an interference filter (1160), another retaining ring (1162), a glass filter (1164), and an additional retaining ring (1166). Detector unit (800) also comprises a set screw (1168) and a photodiode (1170). Retaining rings (1166), (1162), and (1154) are configured to secure the optical components of detector unit (800), as well as to maintain precise clearance between the optical components. While retaining rings (1166), (1162) and (1154) are round, retaining rings in other optical systems may vary in shape and size. Additionally, when multiple retaining rings are used, the retaining rings may all have the same size and/or shape, or at least some of the retaining rings may have different sizes and/or shapes. Other components that are not in the form of rings may still be used to provide a retaining function. Such components may have any suitable shape. For example, lens holder (1158), which helps to hold lens (1156) in place, has a generally tubular shape. While not shown here, some variations of lens holders may have an external surface that is threaded (e.g., to allow for installation into a housing of a detector unit, and/or for adjustment of the position of the lens that is being held).

Glass filter (1164) and interference filter (1160) may be selected, for example, depending on the emission spectrum of the fluorophore or fluorophores in the test strip. The glass filter and interference filter may have fluorophore-tuned spectral qualities. Glass filter (1164) may reduce the intensity of scattered laser light captured by the detectors, and may be any type of optical filter with appropriate transmission characteristics. In some variations, glass filter (1164) may be a red glass filter, such as RG665, RG695, RG830 or other similar filters. Alternatively, a filter made of a plastic or polymeric material which is doped with a dye may also possess the required transmission characteristics, and may be included in the detector unit. Interference filter (1160) may act to further tune and narrow the spectra of light transmitted to lens (1156), with little or no absorption of the transmitted or reflected wavelengths of interest.

In some variations, other optical components may alternatively or additionally be used, such as dichroic filters, glass filters (as previously described), and the like. Additionally, certain variations of detector units may have only one spectral component, or more than two spectral components. The number and type of components may be driven, for example, by the emission spectrum of the fluorophore of interest.

After glass filter (1164) and interference filter (1160) have filtered the emission spectrum from the fluorophore, the filtered emission spectrum is then focused by lens (1156) onto photodiode (1170), which is secured on cover mount (1152). The position and alignment of lens (1156) may be adjusted using set screw (1168) depending, for example, on the spectral content of the filtered fluorescent emissions. The position and alignment of lens (1156) may also be adjusted based on any dependence of the focal length (i.e., the distance from lens (1156) to the source of fluorescent emission) on the peak wavelength(s) of the emission spectrum.

Photodiode (1170) may be of any type that is able to precisely and accurately detect the spectral characteristics of any incident light. While a photodiode is described and shown, it should be understood that other light detective devices or substrates may alternatively or additionally be used, including but not limited to any photodiode arrays, charge-coupled device (CCD), such as CCD image sensors, CMOS image sensors, photoconductive cells, photomultiplier tubes, and the like. Photodiode (1170) may convey the information about the detected light via an electrical interface with the control system.

Housing (1150) and cover mount (1152) generally provide a light-tight environment for the optical components of detector unit (800), and may be made of any opaque material of sufficient thickness to prevent transmission of photons therethrough. A light-tight environment reduces optical noise and may increase the signal-to-noise ratio of the optical signal. Housing (1150) may be of any appropriate shape, and cover mount (1152) may be sized and shaped to be tightly coupled and secured to housing (1150). Additionally, and as shown in FIG. 11B, cover mount (1152) may retain and position photodiode (1170).

While not shown here, some variations of detector units may comprise a lens holder (e.g., lens holder (1158)) that provides adequate light shielding without requiring a housing (e.g., housing (1150)). Additionally, the detector units may comprise a cover mount (e.g., cover mount (1152)) that is configured to be tightly coupled to the lens holder, adjacent to a retainer (e.g., retainer (1154)). The absence of a housing may allow the detector unit to be relatively small, which may in turn reduce the overall size of the optical module.

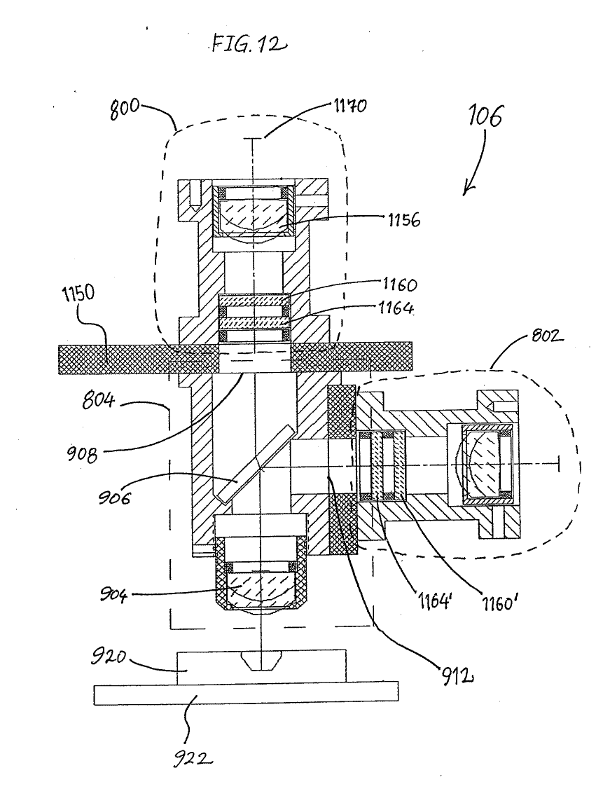

FIG. 12 provides a cross-sectional view of detection module (106), including objective lens unit (804) and detector units (800) and (802). As shown there, and as described above, detector units (800) and (802) are similar, but may have different spectral components. For example, as shown in FIG. 12, detector unit (802) has a glass filter (1164′) and an interference filter (1160′) that may have different spectral filtering characteristics from glass filter (1164) and interference filter (1160) of detector unit (800).

Apertures (908) and (912) of objective lens unit (804) may be configured to allow unobstructed passage of fluorescent emission from the sample in cartridge (920) to detector units (800) and (802). The wavelength of the fluorescent signal that is transmitted through dichroic filter (906) may be tuned for the peak wavelength of the emission spectrum of a first fluorophore, while the wavelength of the fluorescent signal that is reflected by dichroic filter (906) may be tuned for the peak wavelength of the emission spectrum of a second fluorophore.

While certain detection modules have been described, other appropriate detection module configurations may also be used. For example, in some variations, a detection module may include detector units that are substantially parallel to each other, or may include a greater or lesser number of detector units (depending on the range(s) and number of spectra to be detected).

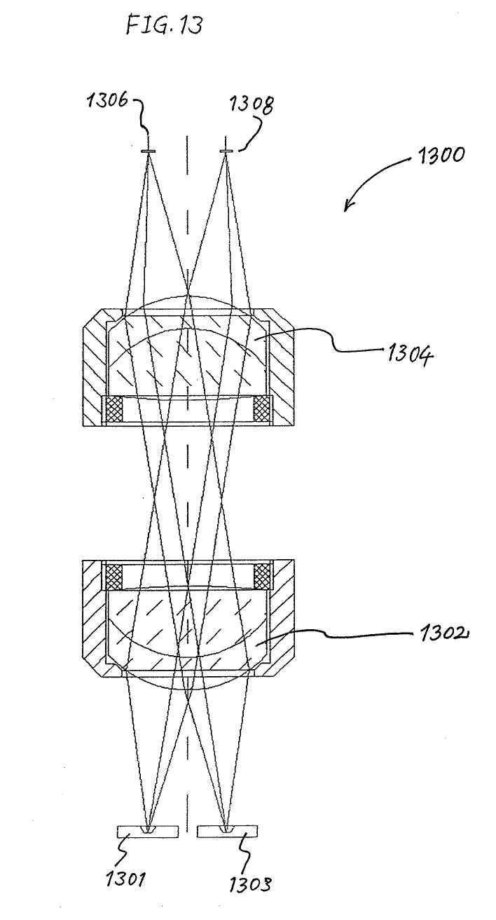

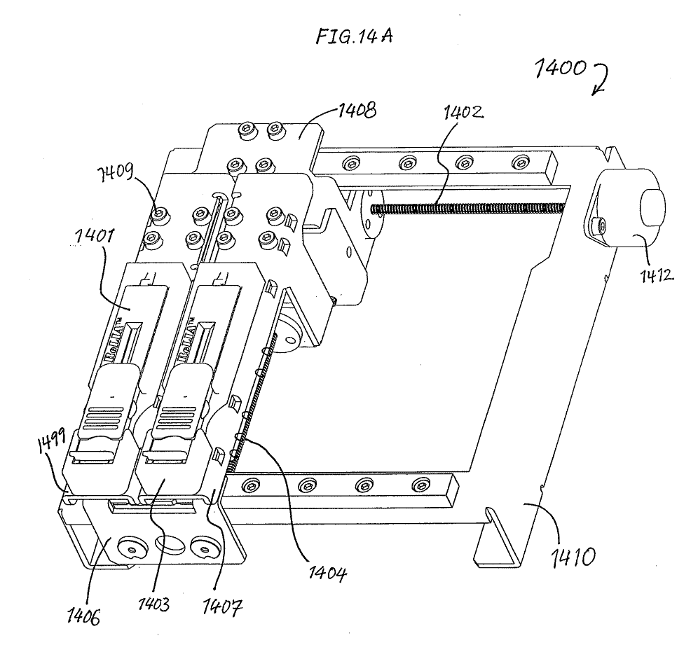

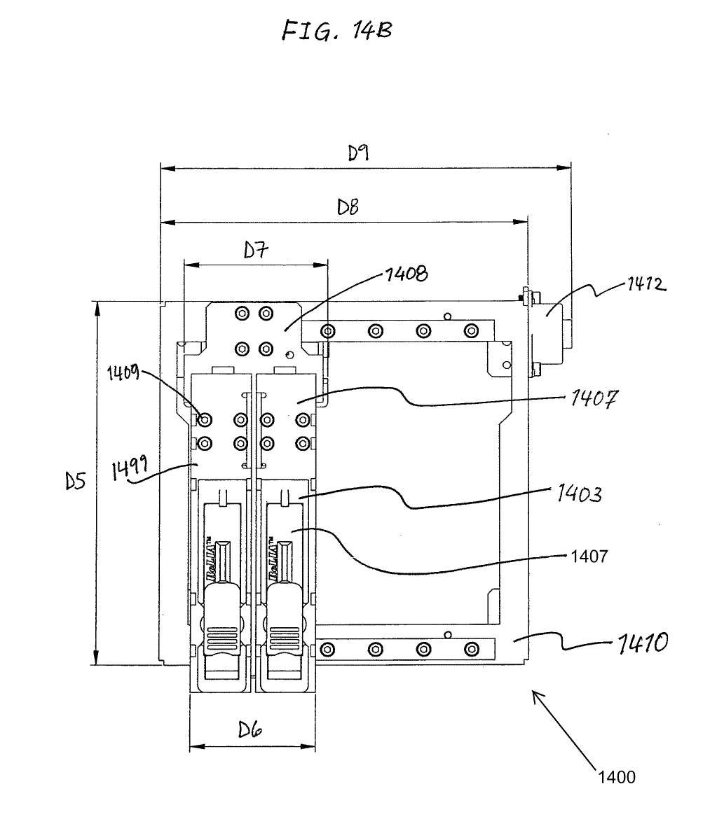

POC diagnostic system (100) (FIG. 1B) is configured to analyze one sample cartridge (111) at a time, with multiple cartridges being analyzed sequentially. However, other variations of diagnostic systems may analyze two cartridges simultaneously, in parallel. For example, FIG. 13 shows a variation of a detection module (1300) which may, for example, be used to simultaneously collect light from two different cartridges. As shown there, fluorescent emission from two cartridges (1301) and (1303) may first be focused through a first lens (1302), and then transmitted through a second lens (1304) that directs the fluorescent signal from each cartridge to a separate sensor. For example, the fluorescent emissions from the sample in cartridge (1303) may be detected by a photodiode (1306), and the fluorescent emissions from the sample in cartridge (1301) may be detected by a photodiode (1308).

While not shown here, some variations of detection module (1300) may comprise one or more glass filters, mirrors, dichroic reflectors and/or achromatic reflectors or refractors, interference filters, and/or other optical components that may provide for the detection and analysis of the emission spectra of more than one fluorophore. For example, to detect and analyze the emissions of a second fluorophore, a dichroic filter may be positioned between lens (1302) and (1304), and may be used to transmit one wavelength to photodiodes (1306) and (1308) and to reflect another wavelength to additional photodiodes positioned orthogonally to photodiodes (1306) and (1308). In some variations, first lens (1302) may be a 1″ objective lens, but any suitable lens type of any size may be used.

Different configurations of detection modules that combine different optical components may be used to reduce the space occupied by the detection module, reduce the cost of the module, or increase the scan efficiency of the system. In some cases, the inclusion or exclusion and/or arrangement of certain optical components may be directed toward decreasing the variability of fluorescent signal detection and increasing its precision.

### Support System

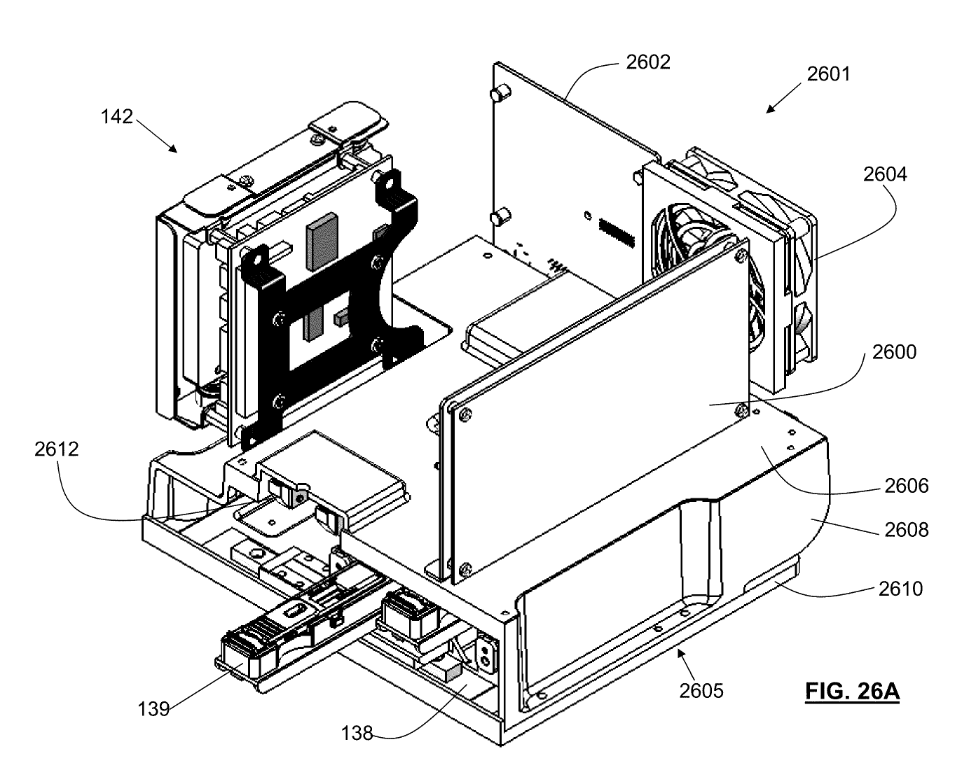

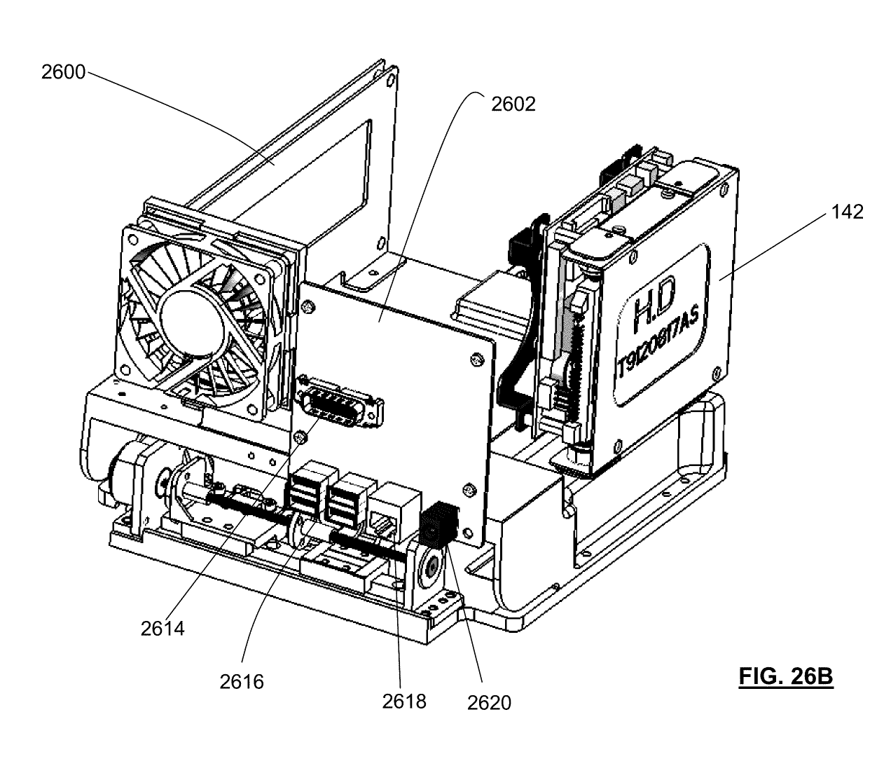

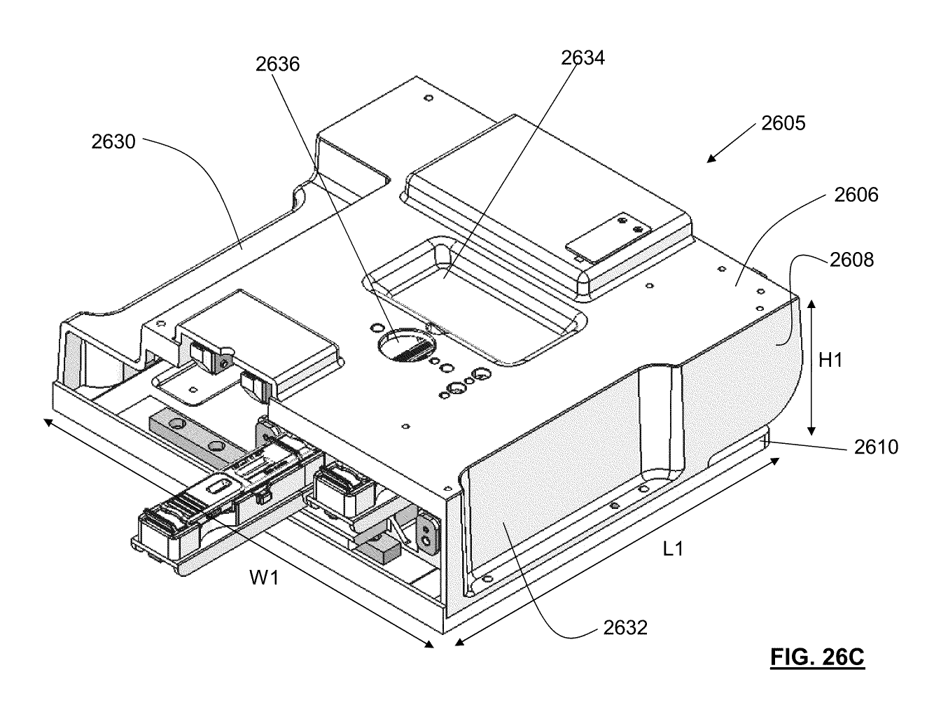

A POC diagnostic system may comprise features that provide structural, electrical, and computational support to the various optical modules described above. For example, an optical module may be mounted and/or secured to a housing or base of a POC diagnostic system such that it has optical access to a test strip. The POC diagnostic system may also comprise computing devices, electrical interfaces, etc., to transmit, receive, and store fluorescent marker emission wavelength data that is collected by the optical module. FIGS. 26A-26C depict one variation of a POC diagnostic system (2601) configuration that may be used with any of the optical modules described above.

POC diagnostic system (2601) may comprise one or more electrical components or interfaces to provide power and data storage capabilities to an optical module. As shown, POC diagnostic system (2601) comprises a mainframe board (2600) that may be used as a relay station between optical module light sensor boards and an embedded computing device (142). For example, emission and/or image data collected by a photodiode of a light sensor board may be transmitted to mainframe board (2600) via a light sensor board connector, and the mainframe board may transmit the data to embedded computing device (142) (e.g., PC 104), via a USB connection. In some variations, mainframe board (2600) may demodulate frequency modulated emission data prior to transmitting to embedded computing device (142).

Some variations of a POC diagnostic system may also comprise a barcode reader or sensor (2612). The barcode reader may be located such that it has access to the barcode of a test strip that has been loaded. The barcode reader may be able to resolve line widths of less than 0.01 inch, and may be able to scan the entire length of the barcode, which may be about 29 mm. In other variations, a POC diagnostic system may have a backscatter device located near or directly under the optical module, which may be configured to sense the backscatter of one (or both) lasers as they are scanned over the barcode. Certain variations of a POC diagnostic system may include one or more devices that can read RFID-tagged test strips. Some POC diagnostic systems may comprise both barcode and backscatter readers and devices.

POC diagnostic system (2601) may also comprise an electrical interface board (2602). Electrical interface board (2602) may comprise a power connector (2620), and multiple types of data connectors, as depicted in FIG. 26B. For example, electrical interface board (2602) may comprise a display connector (2614), one or more (e.g., 2, 3, 4, 6, etc.) USB connectors (2616), and an Ethernet connector (2618). Optionally, electrical interface board (2602) may also have a VGA connector, and may even comprise a device for wireless data transmission. Power connector (2620) may be configured to draw power from a wall socket or other suitable power source, and may draw 100V to 240V AC input, 50-60 Hz. Additionally or alternatively, a battery connector may also be included in the event of an electrical shortage. USB connectors (2616) and Ethernet connector (2618) may provide connectivity to the internet, additional computing devices, and/or other POC diagnostic devices. A mouse and/or keyboard device may be attached to POC diagnostic system (2601) via a USB port (2616). Display connector (2614) may allow data analyses and images to be presented to a monitor or display. In some variations, the display may be touch-sensitive.

As described previously, a POC diagnostic system may also comprise an embedded computing device, such as the one depicted in FIGS. 26A and 26B. Embedded computing device (142) may be any computational processing unit that may be incorporated into a POC diagnostic device. Embedded computing device (142) may also comprise a hard drive or other type of memory, which may be used to store emission data, along with tables and algorithms for analysis.

Referring to FIG. 26A, a cooling element (2604) may also be provided on a POC diagnostic system to help prevent overheating of the system. As shown there, cooling element (2604) may be a fan that is configured to remove heat generated by the optical module and electrical components. In some variations, the operation of cooling element (2604) may be computer controlled using a thermosensor. This may help to maintain a controlled temperature within the system, and help to avoid device overheating, and/or contribute to incubation of test strips. While a single cooling element (2604) is depicted here, it should be understood that other variations of a POC diagnostic system may have two or more cooling elements at different locations in the system which may help to maintain an even temperature within the system.

The optical module, electrical components and cooling components, may be mounted on top of a tray housing (2605). Movable tray (138) may be at least partially enclosed in tray housing (2605). As shown in FIG. 26A and 26C, tray housing (2605) may comprise a top casting (2606), a side casting (2608), and a bottom casting (2610). Top, side, and bottom castings may be individual components that are coupled together, or may be integrally formed, for example, by overmolding or injection molding. Referring to FIG. 26C, tray housing (2605) may comprise a number of apertures, protrusions, grooves, recesses, indentations, and the like that may be used to retain the position of the system components described above with respect to each other. For example, top casting (2606) may comprise a recess (2634) that may be sized and shaped to accommodate the base of an optical module, an aperture (2636) which provides optical access between the optical module and a test strip, and one or more holes that may be threaded to accommodate screws for the attachment of various components (e.g., optical module, cooling element, electrical interface board, etc.). Side casting (2608) may also comprise a first recess (2630) that may be sized and shaped for accommodating an embedded PC, and a second recess (2632) that may be configured to accommodate a mainframe board. Tray housing (2605) may have a length (L1), a width (W1), and a height (H1). Length (L1) may be about 220 mm, width (W1) may be about 220 mm, and height (H1) may be about 50 mm. In other variations, the dimensions of a tray housing may vary. For example, length (L1) may be from about 200 mm to about 400 mm, width (W1) may be from about 200 mm to about 600 mm, and/or height (H1) may be from about 100 mm to about 200 mm.

### Movable Tray

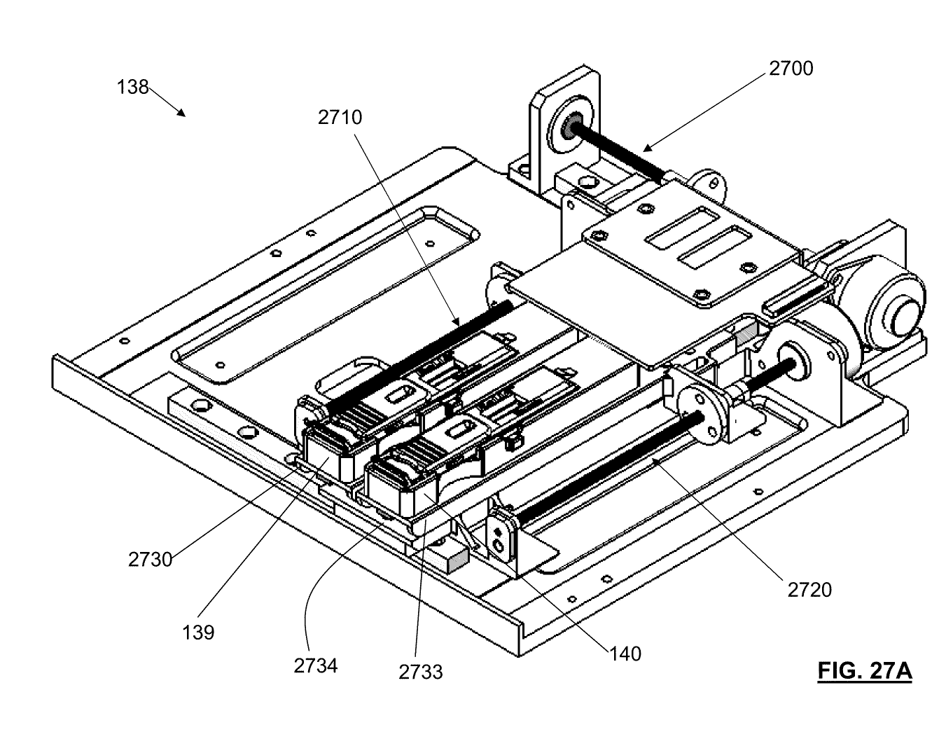

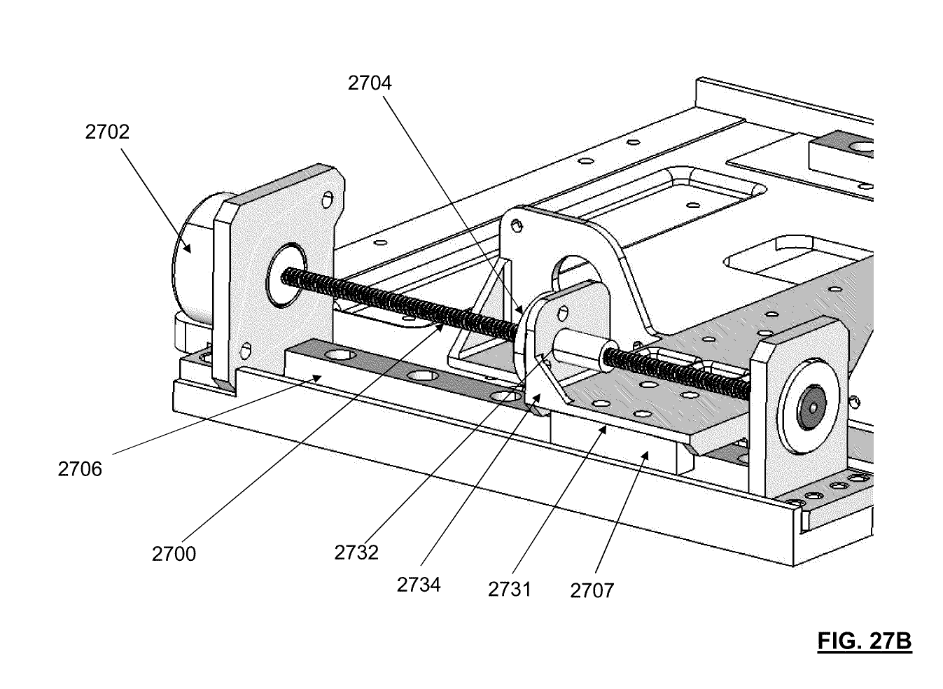

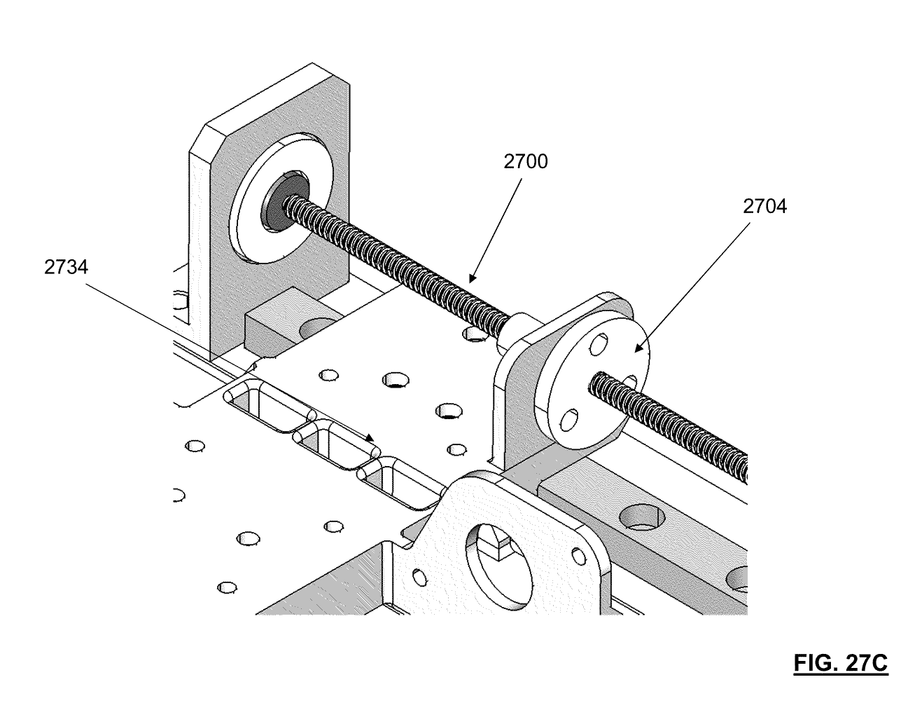

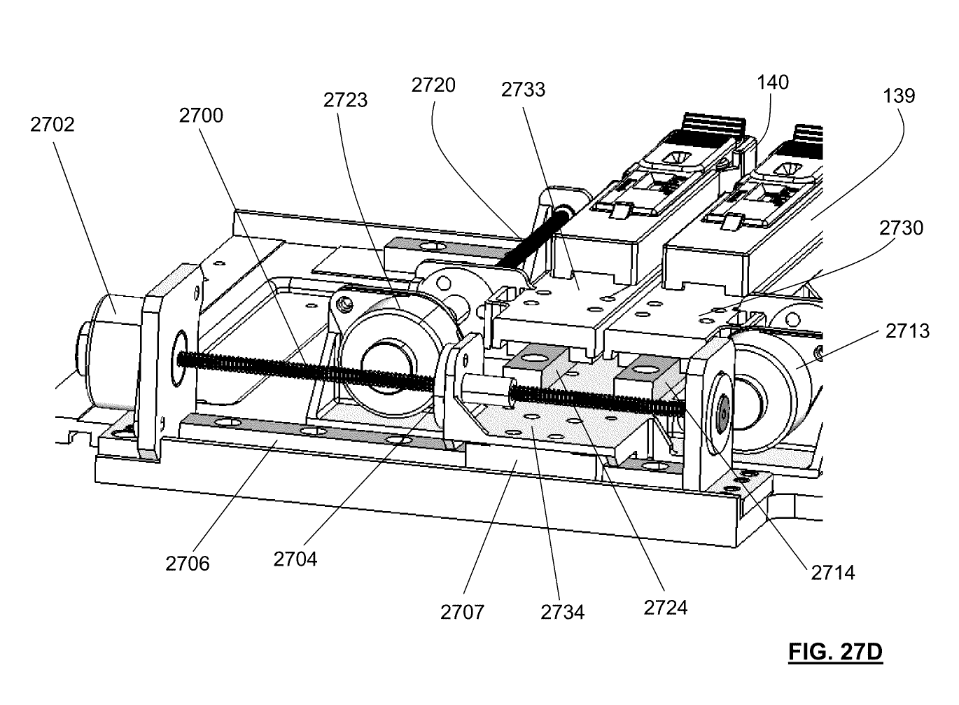

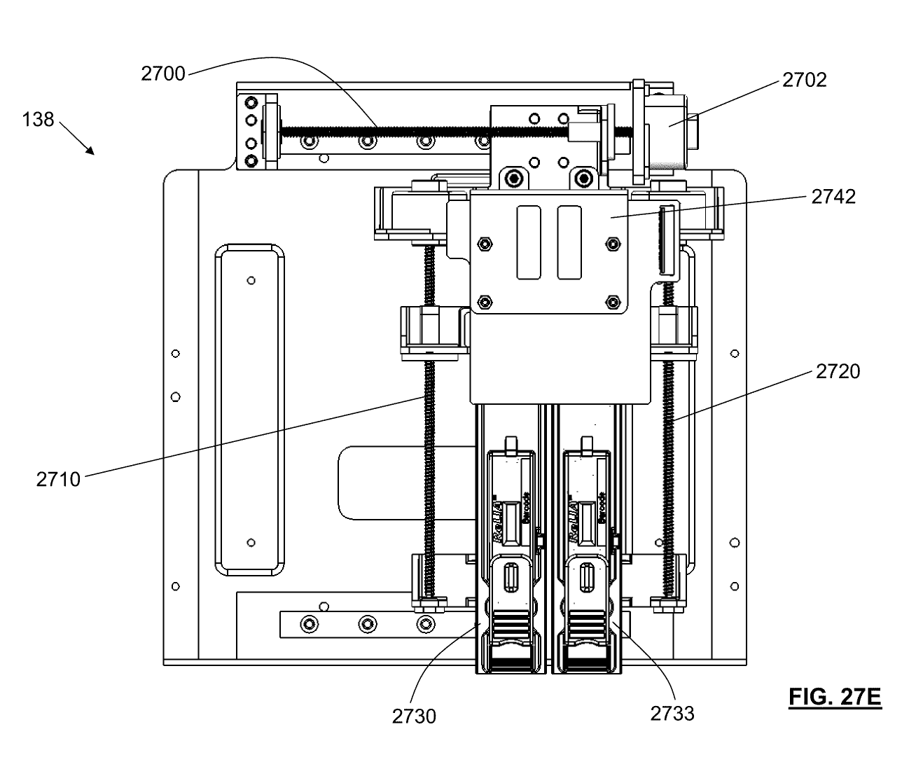

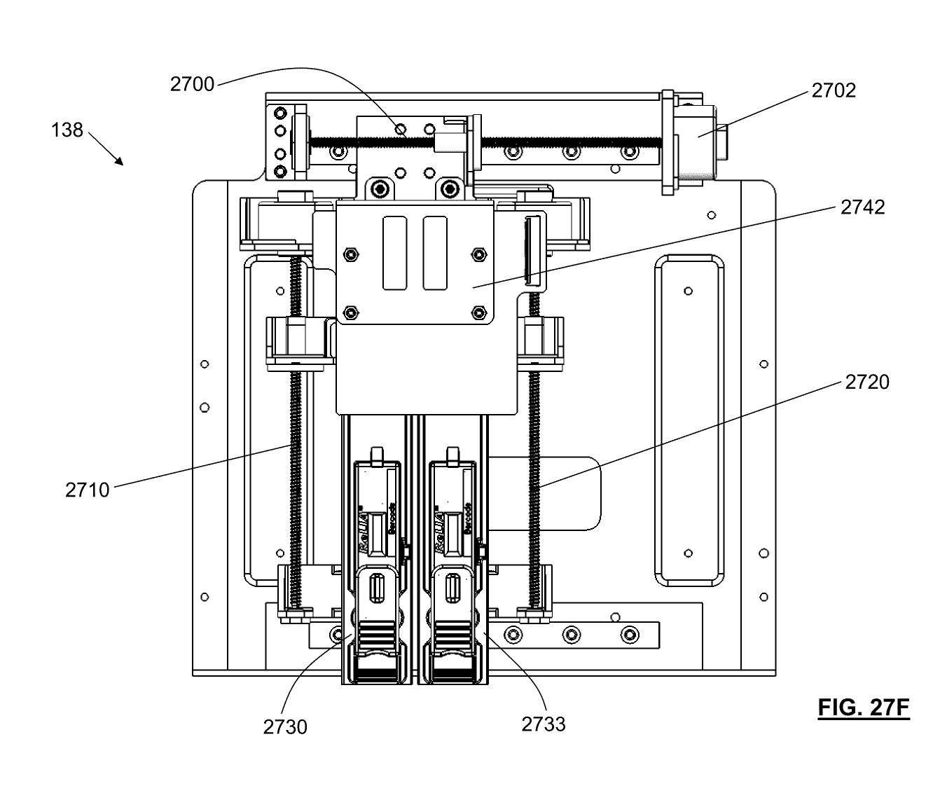

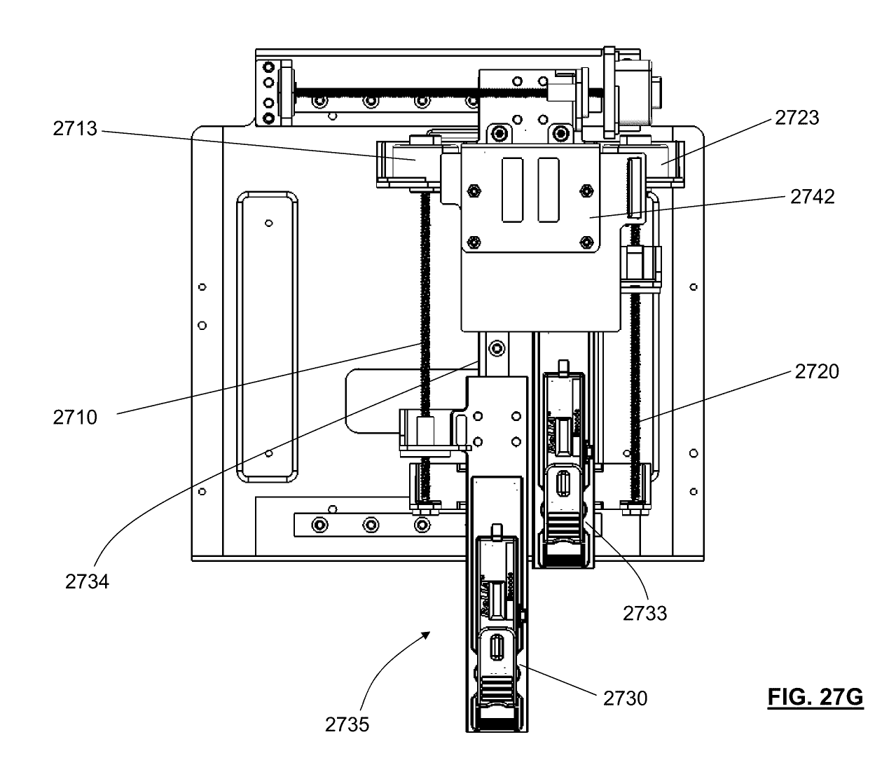

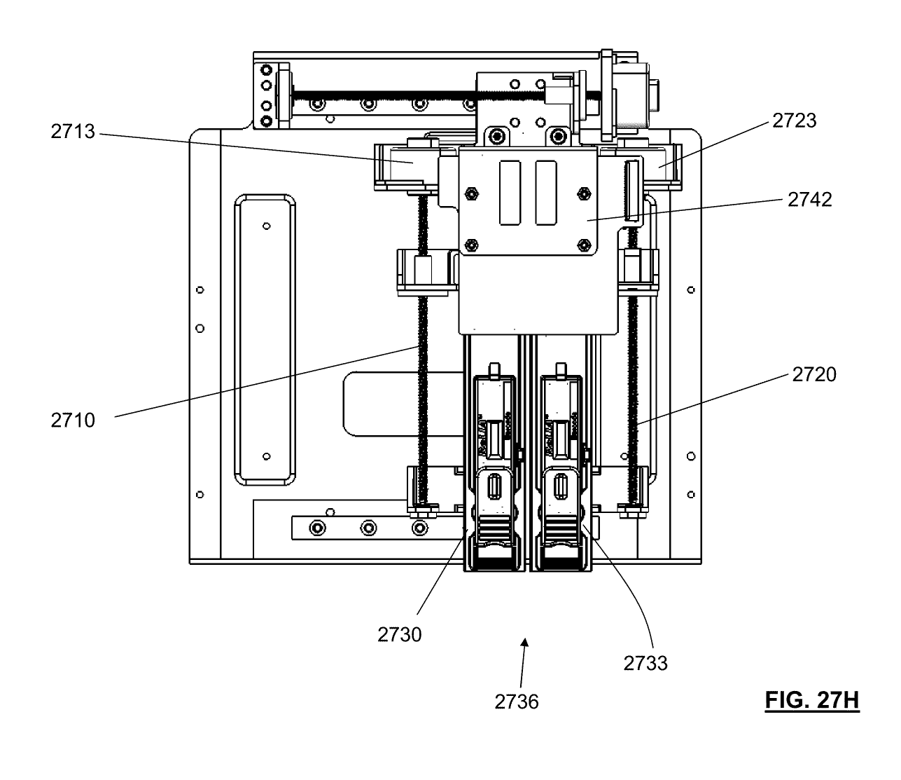

A POC diagnostic detection system may comprise a movable tray that is configured to accept one or more test strips to present to the optical module for testing. A movable tray may be controlled by a computing device or a practitioner to adjust the direction and speed at which the test strips are moved. A movable tray may be configured to position the tray for test strip loading, test strip incubation, and test strip scanning. One example of a movable tray (138) (from system (120) of FIG. 1A) is depicted in FIG. 27A. As shown there, movable tray (138) comprises a horizontal rail (2700), a first transverse rail (2710), a second transverse rail (2720) parallel to the first transverse rail, a first sample stage (139) mounted on a first tray plate (2730) movably coupled to first transverse rail (2710), a second sample stage (140) mounted on a second tray plate (2733) movably coupled to second transverse rail (2720), and a tray base (2734) coupled to horizontal rail (2700), where the first and second tray plates and first and second transverse rails are mounted on the tray base. The length of the horizontal rail and the two transverse rails define the boundaries of movement of sample stages (139) and (140). For example, first sample stage (139), which is mounted on first tray plate (2730), may move along first transverse rail (2710), and second sample stage (140), which is mounted on second tray plate (2733), may move along second transverse rail (2720) independently of the first sample stage and tray plate. The first and second sample stages and tray plates may move together in the horizontal direction according to the movement of tray base (2734) along horizontal rail (2700). In the configuration shown here, the first and second sample stages and tray plates move in concert in the horizontal direction, but in other variations, the first and second sample stages and tray plates may move independently in the horizontal direction. Mechanisms by which a sample stage and tray plates on movable tray (138) are moved horizontally and transversely are described below.

An enlarged view of one variation of a movement mechanism is depicted in FIGS. 27B and 27C. Horizontal rail (2700) has a threaded surface, and may be coupled to a horizontal motor (2702) such that when the motor rotates, the horizontal rail also rotates. A washer (2704) may be fixedly attached to tray base (2734) via an aperture (2732). In some variations, washer (2704) may be a thrust washer. Washer (2704) may be inserted through aperture (2732) and secured with any suitable method (adhesive, soldering, welding, etc.) such that washer (2704) does not rotate. An internal surface of washer (2704) may be threaded, where the threads are complementary to the horizontal rail threaded surface. When horizontal motor (2702) rotates horizontal rail (2700), the rotational motion of the rail and the internal threaded surface of washer (2704) cause washer (2704) to travel along the threads of horizontal rail (2700). Washer (2704) may exert a force upon tray base (2734) to urge it to travel along horizontal rail (2700). To help ensure a straight course of movement, in some variations, a rear portion (2731) of tray base (2734) may be fixedly mounted on a rear linear block (2707) (and similarly, a front portion of the tray base may be fixedly mounted on a front linear block), which may be slidably coupled with a horizontal linear guide (2706). The linear block may have a slot sized and shaped to retain the linear guide. In some variations, the linear block may have a set of circulating ball bearings on each side of the slot. The ball bearings may ride in a small slot on each side of the linear guide (not shown). Activating horizontal motor (2702) to rotate in a first direction may cause horizontal travel of tray base (2734) in a first horizontal direction, and activating the motor to rotate in a second direction may cause horizontal travel of the tray base in a second horizontal direction. The first and second sample stages and tray plates that are mounted on tray base (2734) move horizontally in accordance with the movement of the tray base.

Transverse movement of the first and second sample stages and tray plates (e.g., along first and second transverse rails (2710) and (2720)), may be actuated using a similar mechanism. One way in which first and second sample stages and tray plates may move both horizontally and transversely is depicted in FIG. 27D. The configuration depicted there allows the transverse movement of the first sample stage and tray plate to be independent from the transverse movement of the second sample stage and tray plate, however, in other variations, first and second sample stages and tray plates may be configured to move together. As shown in FIG. 27D, a first transverse motor (2713) and the first transverse rail (not shown) may be mounted along a first long edge of tray base (2734), and a second transverse motor (2723) and second transverse rail (2720) may be mounted on the opposite long edge of tray base (2734). The first and second transverse rails may be threaded similar to the horizontal rail. A first transverse linear guide (2714) may be mounted parallel to the first long edge of tray base (2734), just inside and parallel to the first transverse rail, and similarly, a second transverse linear guide (2724) may be mounted parallel to the opposite long edge, just inside and parallel to second transverse rail (2720). First and second tray plates may be movably coupled to the first and second transverse rails using threaded washers, and mounted over the first and second transverse linear guides using linear blocks as described above.

During use, first tray plate (2730) may move transversely along first linear guide (2714) by activating the rotational motion of first transverse motor (2713). Similarly, second tray plate (2733) may move transversely along the second linear guide (2724) by activating the rotational motion of second transverse motor (2723). Horizontal movement of tray base (2734) moves the first and second linear guides horizontally, which in turn moves the first and second tray plates horizontally. While one movement mechanism is described and depicted here, other mechanisms and configurations may be implemented to provide both horizontal and transverse movement of the tray plates to incubate and position the test strips for scanning and analysis.

FIGS. 27E-27I depict the various configurations that tray plates (2730) and (2733) may assume during use. In the variation of movable tray (138) shown here, tray plates (2730) and (2733) move in concert along the horizontal direction; however, in other variations, tray plates (2730) and (2733) may be configured to move independently of each other along the horizontal direction. In FIG. 27E, tray plates (2730) and (2733) are in a rightmost horizontal location, while in FIG. 27F, they are in a leftmost horizontal location. During use, test strips retained by sample stages (139) and (140) mounted on tray plates (2730) and (2733) may be in the leftmost horizontal location during the incubation of the fluid sample, for example. Once the desired incubation period has lapsed, tray plates (2730) and (2733) may be actuated to move to the rightmost horizontal location for detection of fluorescent emissions (i.e., test strip scanning). Tray plates (2730) and (2733) may be actuated to any location along horizontal rail (2700) which may be suitable for presenting a test strip for scanning by the optical module.

The movement of tray plates (2730) and (2733) may be computer controlled, pre-programmed, or user controlled, as appropriate. Commands may be issued to activate the horizontal as well as vertical motors via a control interface (2742). Control interface (2742) may be configured to accommodate substantially planar electrical connectors, which may reduce the interference of the connectors with the movement of the tray plates and tray base. There may be one or more control interfaces (e.g., 1, 2, 3, 5, etc.), as appropriate for providing electronic control to the various motors. The movement and location of tray plates (2730) and (2733) during a test strip scan may be coordinated with the activation of the excitation module of the optical module (e.g., to read fluorescent marker emission data along a scan line by stepwise or incremental movement of the test strips located on tray plates (2730) and (2733)). The position of tray base (2734) along horizontal rail (2700) may be determined by maintaining a count of the number of turns the motor has rotated, or by using a position sensor, which will be described below.

Tray plates (2730) and (2733) are each coupled to separate transverse rails. More specifically, the movement of first tray plate (2730) is coupled to the activation of first transverse motor (2713) and rotation of first transverse rail (2710), while the movement of second tray plate (2733) is coupled to the activation of second transverse motor (2723) and rotation of second transverse rail (2720). FIGS. 27G-27I depict exemplary transverse configurations of first tray plate (2730), while keeping second tray play (2733) in the same position. FIG. 27G depicts first tray plate (2730) in a protruded configuration (2735), which may be suitable for the loading and removal of a test strip cartridge. FIG. 27H depicts first tray plate (2730) in a middle configuration (2736), which may be suitable for translating the tray plate along the horizontal direction to transition it between an incubation configuration and a test strip scanning configuration. FIG. 27I depicts first tray plate (2730) in a retracted configuration (2737), which may be suitable as a test strip on first tray plate (2730) is scanned by the optical module. Second tray plate (2733) may also move transversely, independently from the movement of first tray plate (2730). In other variations, first tray plate (2730) and second tray plate (2733) may be configured such that their movement in the transverse direction is in concert. Various degrees of freedom for each of the tray plates may be implemented as desirable for the loading, incubating, and scanning of test strips. In some variations, the rate of tray plate and tray base movement may be programmable, computer or user controlled. For example, the tray plate and the tray base may move horizontally or transversely at a rate of about 20 mm/second to about 40 mm/second. In some variations, a tray plate or tray base may be moved at a rate such that a test strip is scanned in less than 1 second.

While movable tray (138) is depicted has having two tray plates (2730) and (2733), other variations of movable trays may have any number of tray plates to retain any number of test cartridges. For example, a movable tray may have 1, 3, 4, 5, 8, 10, etc. tray plates. The number of horizontal and/or transverse rails may be determined in part by the number of tray plates in the movable tray. Other variations of movable trays may position the tray plates in, for example, a carousel, a rotatable wheel, or another circular and/or non-planar structure. This may help to increase the number of tray plates retained by a movable tray.

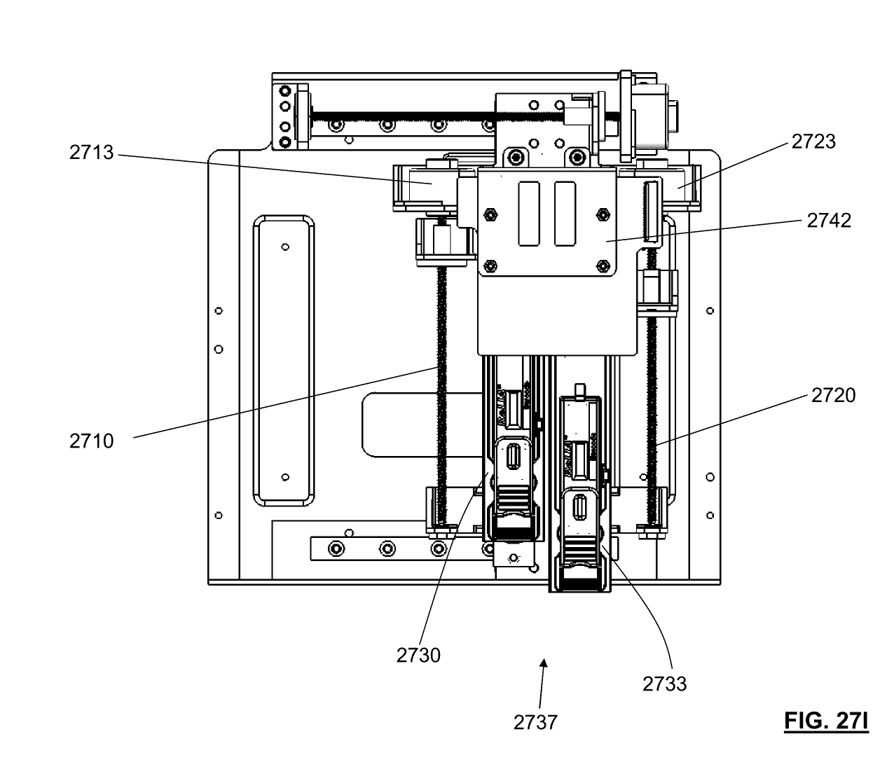

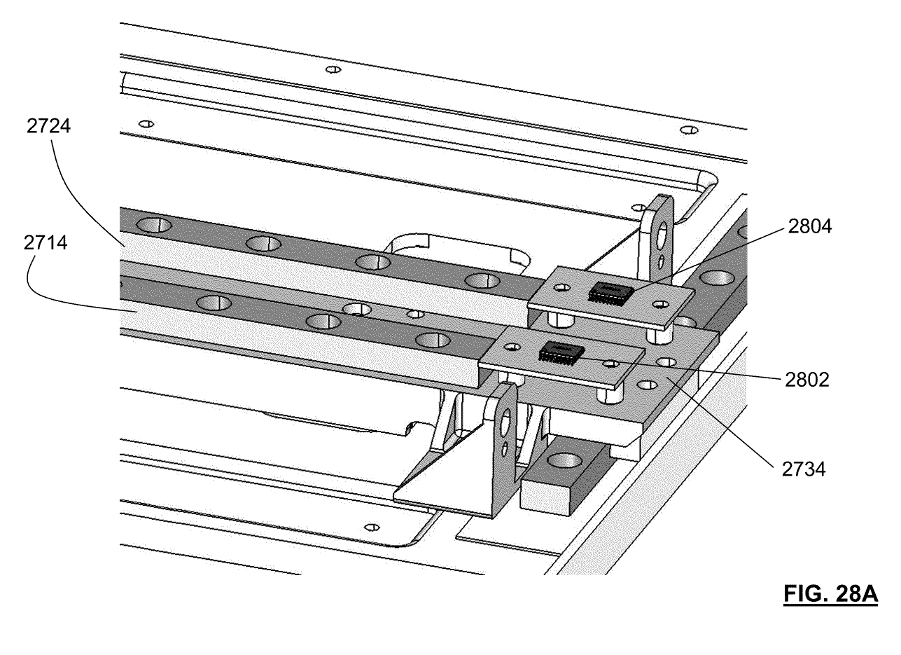

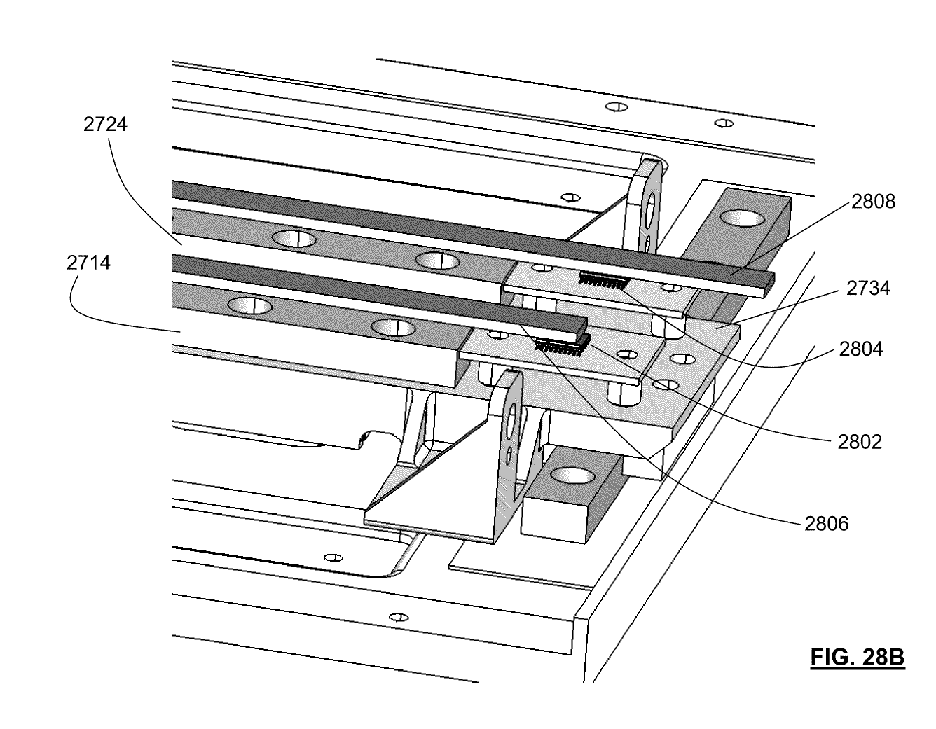

A movable tray of a POC diagnostic system may use various mechanisms to monitor the location of a tray base or tray plate. For example, optical encoders may be used to detect the location of a tray base or tray plate. One example of a magnetic mechanism that may be used to monitor the transverse movement of first and second tray plates is depicted in FIGS. 28A and 28B. As described previously, first and second tray plates are movably coupled to first and second linear guides (2714) and (2724), and slide over them according to the rotation of the first and second transverse motors. In some variations, a first magnetic motion encoder (2802) and a second magnetic motion encoder (2804) may be mounted on one end of tray base (2734), as shown in FIG. 28A. First and second magnetic motion encoders may be in the form of integrated circuits that sense the motion of a multi-pole magnetic strip or ring; for example, they may be high resolution magnetic linear encoders such as AS5311. In some variations, an integrated circuit may utilize integrated Hall elements, analog elements, and a digital signal processing element. For example, magnetic motion encoders may provide a serial bit stream output to an embedded computing device (e.g., via a control interface such as control interface (2742) to control the motion of the tray plates according to a pre-programmed or user-determined sequence.

A multi-pole magnetic strip may be embedded with first and second tray plates, such that movement of the tray plates may be tracked according to the movement of the embedded magnetic strip. FIG. 28B depicts a first multi-pole magnetic strip (2806) that may be embedded in a first tray plate, and a second multi-pole magnetic strip (2808) that may be embedded in a second tray plate. The multi-pole magnetic strip may have any suitable pole arrangement. One example of a magnetic strip that may be used here is multi-pole magnetic strip MS 10-10, with a pole length of 1.0 mm and 10 poles. While a magnetic movement sensor has been described here, other movement and/or position sensing mechanisms may be used, such as accelerometers, acoustical methods, optical methods, etc. In some variations, a movable tray may have end limit sensors that may help to increase positional precision.

### Sample Stage

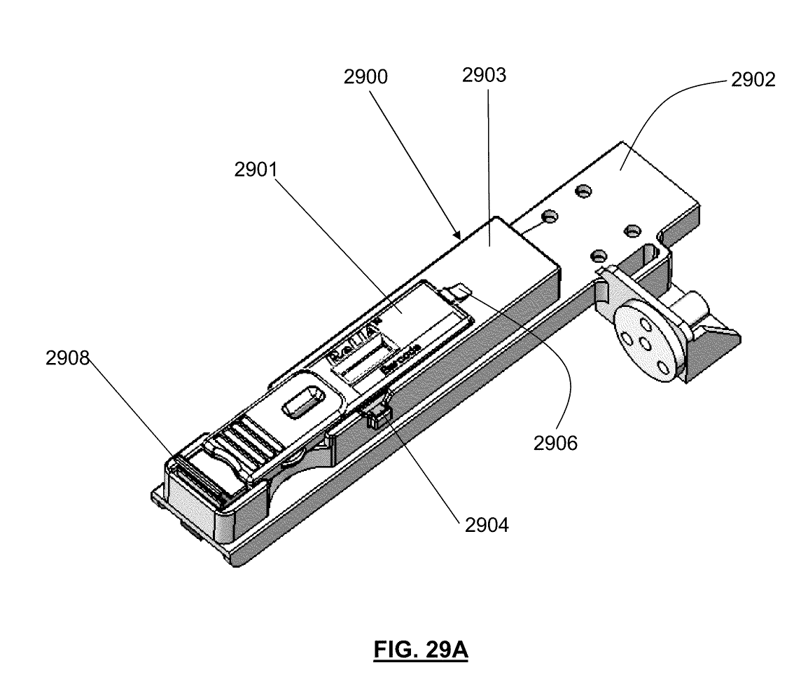

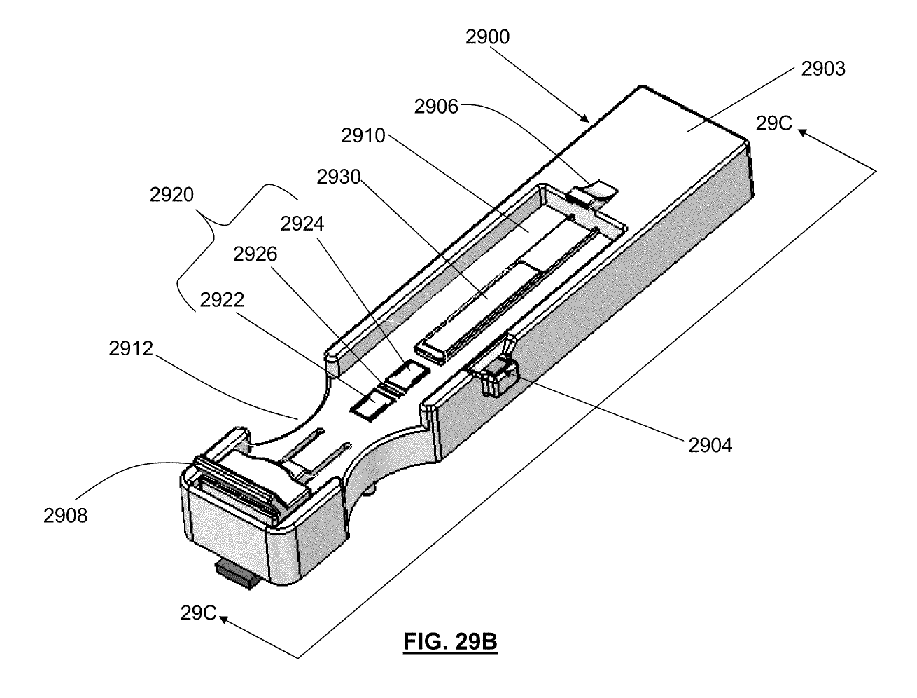

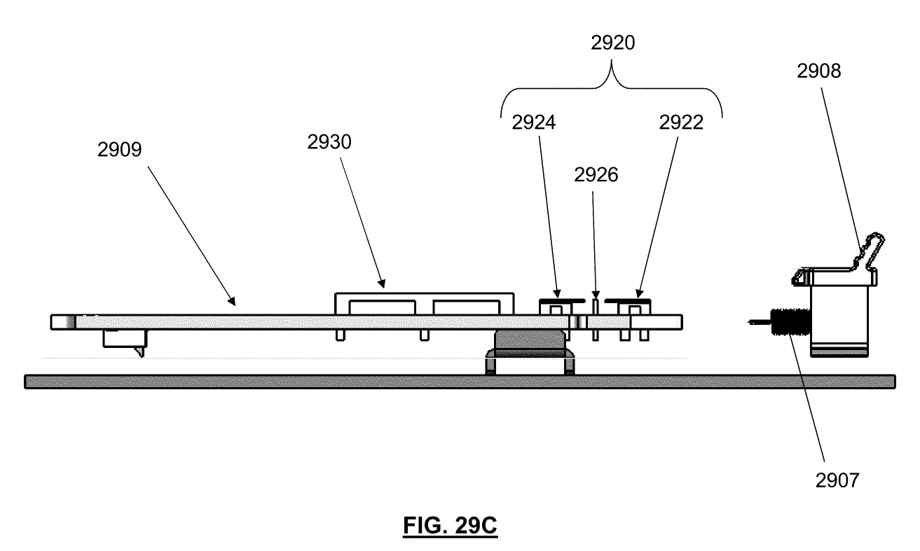

Depending on the fluid sample to be tested, and the targeted analyte(s), a test cartridge containing a fluid sample may require different incubation conditions, such as different amounts of time, temperature, etc. Some variations of diagnostic systems may comprise elements that regulate the temperature and/or humidity of the incubation environment. In the variation of a diagnostic system described here, the sample stage and/or tray plate may comprise temperature and fluid sensors, heating elements, and retaining elements that may help improve the speed and precision of a diagnostic test. One example of a sample stage (2900) that is configured to retain a test cartridge (2901) is shown in FIGS. 29A-29C. FIG. 29A depicts sample stage (2900) mounted on a tray plate (2902). Tray plate (2902) may be similar to the tray plates previously described. As shown in FIG. 29A, sample stage (2900) comprises a stage housing (2903) with a proximal flange (2906) and a distal flange (2908), where the distance between the proximal and distal flanges may be suitable for accommodating a test cartridge (2901). Stage housing (2903) may have any number, size, and shape of grooves, protrusions, recesses, notches, flanges, and the like to securely retain test cartridge (2901) during incubation and scanning, as well as to allow a practitioner to disengage test cartridge (2901) at the conclusion of the test analysis.

FIG. 29B illustrates sample stage (2900) without test cartridge (2901). As shown there, stage housing (2903) comprises a cartridge recess (2910) sized and shaped to releasably retain a cartridge. Proximal flange (2906) and distal flange (2908) may be deflectable so that a test cartridge may be snap-fit into cartridge recess (2910). Optionally, a spring (2907) (FIG. 29C) may be provided at the distal end of cartridge recess (2910), and may exert a compressive force on a cartridge placed within the cartridge recess. While one variation is shown here, any suitable retaining structure may be used to releasably engage a test cartridge for testing. The stage housing may also comprise one or more curved indentations (2912) that allow for ergonomic engagement and disengagement of a test cartridge. The geometry of cartridge recess (2910) may be such that the bottom portion of a test cartridge engaged in the sample stage is in substantial contact with the bottom surface of cartridge recess (2910). In the variation of a sample stage described here, sample stage (2900) also comprises a fluid sensor (2920) and a heating element (2930). Each of these components is described in detail below.

Fluid sensor (2920) is configured to detect the addition of a fluid sample, which may then signal the movable tray system to automatically draw the tray inwards, and start the incubation timer. This may help to ensure precise incubation timing between samples. As depicted in FIG. 29B, fluid sensor (2920) comprises a transmit element (2922), a receive element (2924), and a shield (2926) disposed between the transmit and receive elements, where the transmit element, the receive element and the shield are embedded in a PCB board (2909) (FIG. 29C). FIG. 29C is a partial cutaway side view of sample stage (2900), with a portion of stage housing (2903) removed.

Transmit element (2922) may be any device configured to transmit a modulated radiowave, such as an audio tone or any modulated electromagnetic signal. For example, transmit element (2922) may be an oscillator. Transmit element (2922) and receive element (2924) may be configured to measure changes in the dielectric property of a material that spans the distance between the transmit and receive elements. For example, the dielectric property of a dry sample pad changes when a fluid sample is applied to it, and this change may be detected by the transmit and receive elements. Fluid sensor (2920) may signal the presence or absence of a fluid sample in a test cartridge by generating a signal that may be transmitted to an embedded computing device, which may generate a visual, audio, or other indicator or alarm.

As shown, sample stage (2900) also comprises a heating element (2930), which may be used to adjust the temperature in the immediate proximity of a test cartridge. This may help analyte binding agents, analyte capture agents, and any fluorescent markers to react and/or bind with the targeted test analyte. It may also increase the rate of lateral flow of the fluid sample between the bands and pads of a test strip. Cooling elements may also be included as desired. Additionally, sample stage (2900) may comprise a temperature sensor near the heating element. Heating element (2930) may be heated by, for example, resistive heat generated by circuits on PCB board (2909). Other heating features may be included here, as well as other methods of expediting analyte binding. Moreover, in some variations, a sample stage may include a cooling bar or other cooling element that functions to reduce the temperature (i.e., to act as a cooler). This may, for example, expedite analyte binding and/or prevent evaporation of fluid from the test strip (or other test medium). For example, in a hot environment the cooling element may reduce the temperature. In general, a heating element, or a cooling element, may comprise any feature or features that adjust the temperature on the test strip to a temperature range suitable for effective analyte binding and/or for preventing fluid evaporation from the test strip or other test medium. It should also be noted that some variations of sample stages may not comprise any heating elements, cooling elements, and/or temperature sensors.

As illustrated in FIGS. 29A and 29B, sample stage (2900) may also comprise a laser calibration glass (2904) that may be used to calibrate the output power and/or intensity of the laser beams emitted from the excitation module. Laser calibration glass (2904) may be, for example, polished didymium glass or glass containing ions of rare earth elements, which may be suitable for calibrating excitation detection modules that are configured to emit and detect light in the red and infrared region of the spectrum. Laser calibration glass (2904) may be located on a surface of stage housing (2903) that may be moved to coincide with the laser beam path of the excitation module, as well as coincident with the optical axis of a detection module objective lens. The dimensions of laser calibration glass (2904) may vary as appropriate, and may be, for example, about 2 mm wide, about 3 mm long, and/or about 1 mm thick. The intensity and/or output power data that is collected by light sensor boards in the excitation and detection modules may be used to electronically regulate the current through the lasers, and may also be used as a feedback signal to a computing system to regulate the power of the lasers of the excitation module. In some variations, the intensity and/or output power data may also be used to dynamically adjust the gain of the photodiode, or the 24 bit analog-to-digital converter on the light sensor boards of the detection module. While the calibration element here may be made of didymium glass, it should be understood that any material with precise and reliable optical properties within the spectrum of the laser beam may be used to calibrate the laser power output.

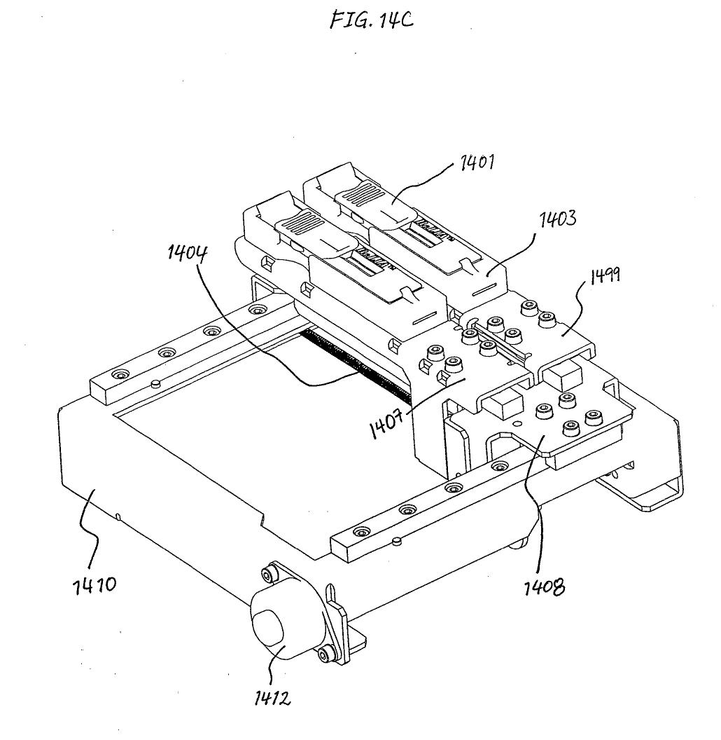

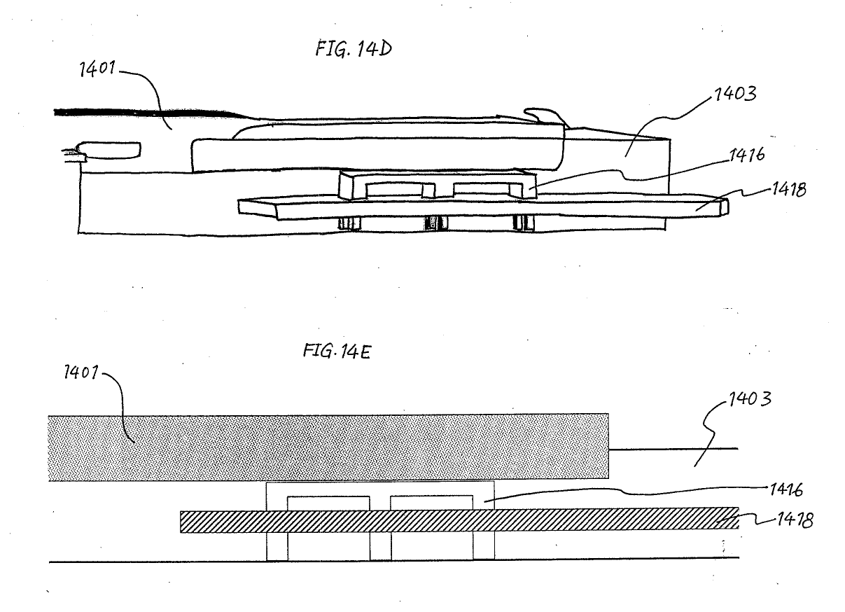

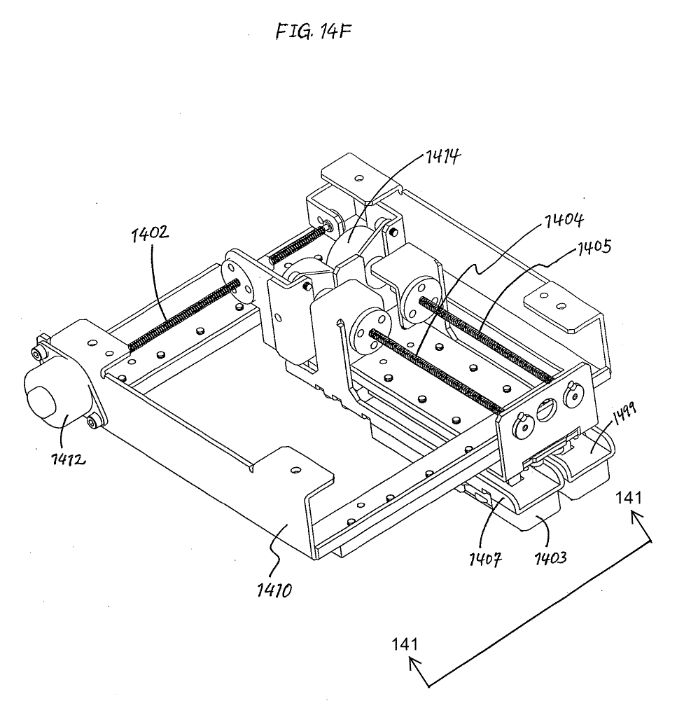

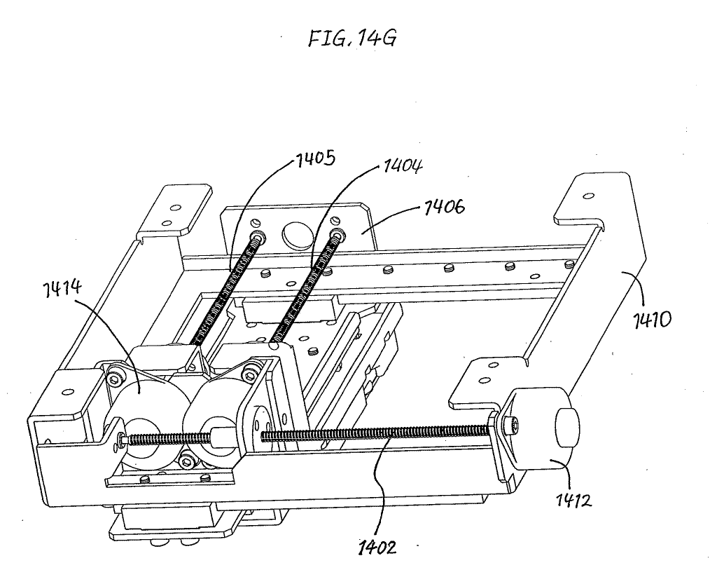

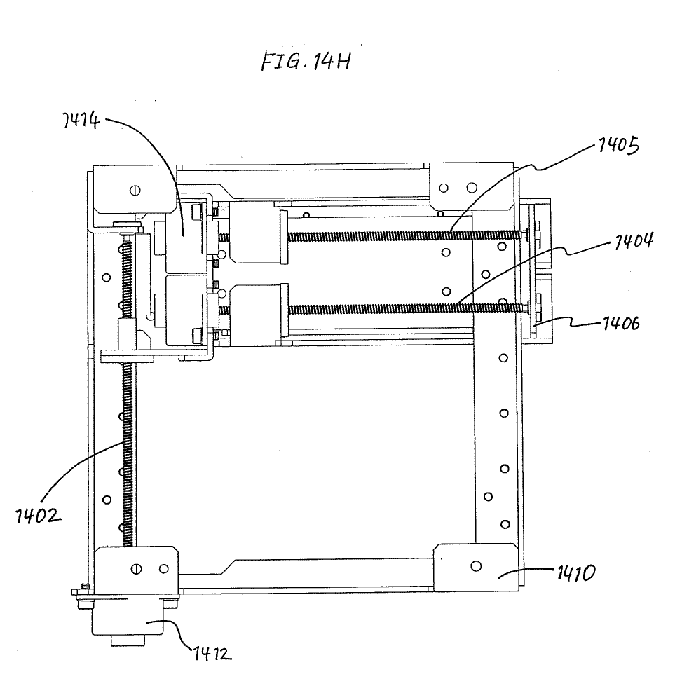

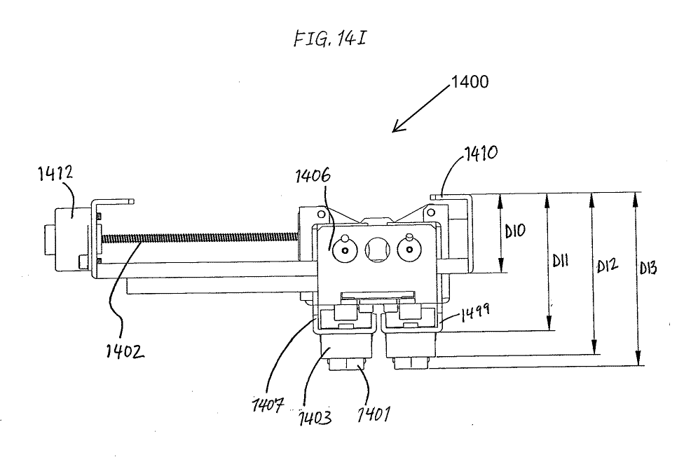

FIGS. 14A-14I depict another variation of a movable or motorized tray drive (1400) which may be used with one or more of the systems described here. More specifically, FIGS. 14A and 14C are perspective top views of tray drive (1400), FIGS. 14D and 14E are perspective and cross-sectional views of a heater bar in the sample holder of the tray drive, FIGS. 14F and 14G are perspective bottom views of tray drive (1400), FIG. 14B is a top view of tray drive (1400), FIG. 14H is a bottom view of tray drive (1400), and FIG. 14I is a side view of tray drive (1400). Tray drives may be actuated to position and align one or more cartridges and test strips for optical detection and analysis. For example, a tray drive may position sample holder (109) so that cartridge (111) is aligned with aperture (112), as shown in FIG. 1B.

Referring again to FIGS. 14A-14I, tray drive (1400) comprises a tray chassis (1410), a chassis rail (1402), tray rails (1404) and (1405), slidable mounts (1406) and (1408), a chassis motor (1412), and at least one tray (1407) comprising a tray motor (1414).

A cartridge (1401) and sample holder (1403) are also depicted. Cartridge (1401) may be secured in sample holder (1403) in any appropriate fashion, including via a snap-fit or friction-fit, and/or using adhesives, magnets, electrostatic force, or compressive forces. As shown in the figures, sample holder (1403) is coupled to tray (1407). Sample holder (1403) may, for example, be a separate component that is coupled to tray (1407) after formation. In other variations, sample holder (1403) may be integrally formed with tray (1407).

As shown in FIG. 14A, tray (1407) is coupled to slidable mounts (1406) and (1408) by a number of screws (1409). Tray (1407) is actuated by a tray motor (1414) via tray rail (1404), as depicted in FIG. 14F. This may allow for movement of tray (1407) along the axis defined by the tray rail (1404). Motor (1414) may be manually or electromechanically actuated. Movement along the axis defined by rails (1404) and (1405) may facilitate the scanning of the sample contained in the cartridge (e.g., by optical module (101) shown in FIG. 1B). As shown in FIG. 14A, tray drive (1400) includes two trays (1407) and (1499) mounted on slidable mounts (1408) and (1406). Tray (1499) may function as described above for tray (1407), for example. It should be understood that other variations of motorized tray drives may include any appropriate number of trays mounted on slidable mounts, such as three, four, five, or ten trays, etc.

Slidable mount (1408) is coupled to chassis motor (1412) via chassis rail (1402). This may allow slidable mounts (1406) and (1408), carrying trays (1407) and (1499), to be moved along the axis defined by chassis rail (1402). Chassis motor (1412) may be manually or electromechanically actuated. Thus, tray drive (1400) has two degrees of freedom: one along the axis defined by chassis rail (1402) and another along the axis defined by tray rails (1404) and (1405). Other variations of tray assemblies may have more or fewer degrees of freedom depending on the number of rails and motors. For example, some variations of trays may not have a tray rail and motor, such that motion of the trays is limited to the axis defined by the chassis rail. In other variations, the trays may have tray motors, but no chassis rail or motor, so that motion of the trays is limited to the axis defined by the tray rails. Chassis rail (1402) and slidable mounts (1406) and (1408) are coupled to the edges of chassis (1410), as shown in FIGS. 14A-14I.

As shown in FIG. 14B, chassis (1410) has dimensions (D5) and (D8), depicted in FIG. 14B. In some variations, dimension (D5) may be approximately 150 mm. Alternatively or additionally, dimension (D8) may be approximately 150 mm. Dimension (D9) is equal to the entire width of tray drive (1400), and in some variations may be approximately 170 mm. Dimension (D7) denotes the width of slidable mount (1408), and in certain variations may be approximately 70 mm. Finally dimension (D6) is equal to the width of tray (1407), and may be approximately 50 mm. The components of tray drive (1400) may be of any size that allows them to be integrated with and supported by chassis (1410).

In some variations of a motorized tray drive, the sample holder (1403) may comprise a heater bar (1416) embedded into a circuit board (1418), as shown in FIGS. 14D and 14E. The heater bar and circuit board may be arranged so that when a cartridge (1401) is placed into sample holder (1403), the heater bar (1416) is in substantial contact with the cartridge. The heater bar may be heated by, for instance, resistive heat generated by circuits on the circuit board (1418) and may act to expedite analyte binding. Other heating and/or cooling features (e.g., a cooling bar) may be included here, as previously described.

Chassis (1410) may comprise, for example, one or more relatively rigid materials that can withstand the weight of optical system (101) or any other optical system suitable for use therewith. In some variations, chassis (1410) may be bolted to a stable surface (e.g., to reduce vibrations that may perturb the system). FIG. 14I depicts a side-view of tray drive (1400). As shown there, chassis (1410) has a depth (D10), which may be, for example, approximately 32 mm. In FIG. 14I, dimension (D13) denotes the total depth of tray drive (1400), the sum of the depths of tray (1407), sample holder (1403), and cartridge (1401). In certain variations, dimension (D13) may be about 70 mm. The dimension (D11) denotes the total depth of chassis (1410) to tray (1499), and dimension (D12) denotes the total depth of chassis (1410) to the bottom of sample holder (1403). Dimensions (D5)-(D13) define the space occupied by this variation of a motorized tray drive, as well as the positioning of the various components with respect to each other, which may contribute to the portability of the overall POC diagnostic system.

In some variations of a diagnostic system, the optical module may be mounted on top of the motorized tray drive, similar to the depiction in FIGS. 1A-1C. Dimensions (D10)-(D13) may provide guidance as to a minimum clearance that may be provided between the optical module and the motorized tray drive so that the optical module does not impede the motion of the tray. The optical module housing (e.g., housing (102)) may comprise one or more features that can be used to couple the optical module to the motorized tray drive without impeding the motion of the trays. These features may include, but are not limited to, apertures, grooves, slots, notches, recesses, and channels. In some variations, there may be an electrical interface between the optical module and the motorized tray drive, so that their operation may be synchronized.

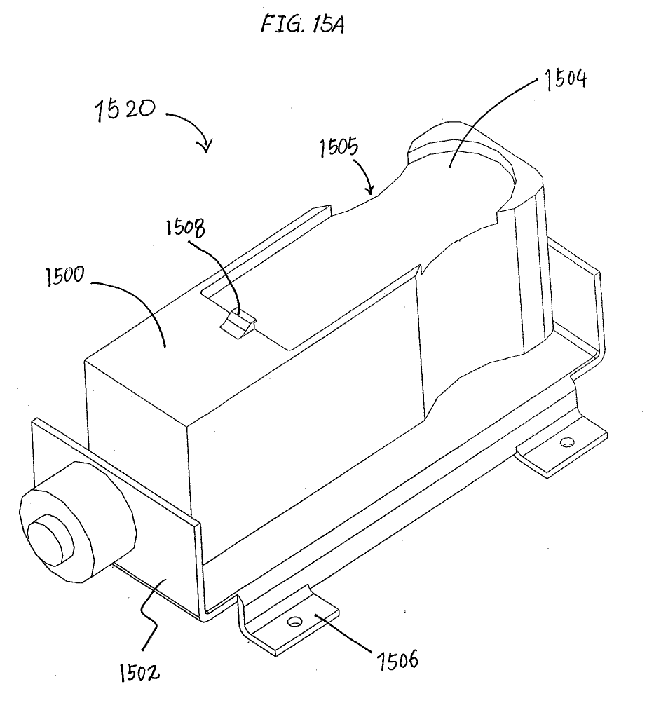

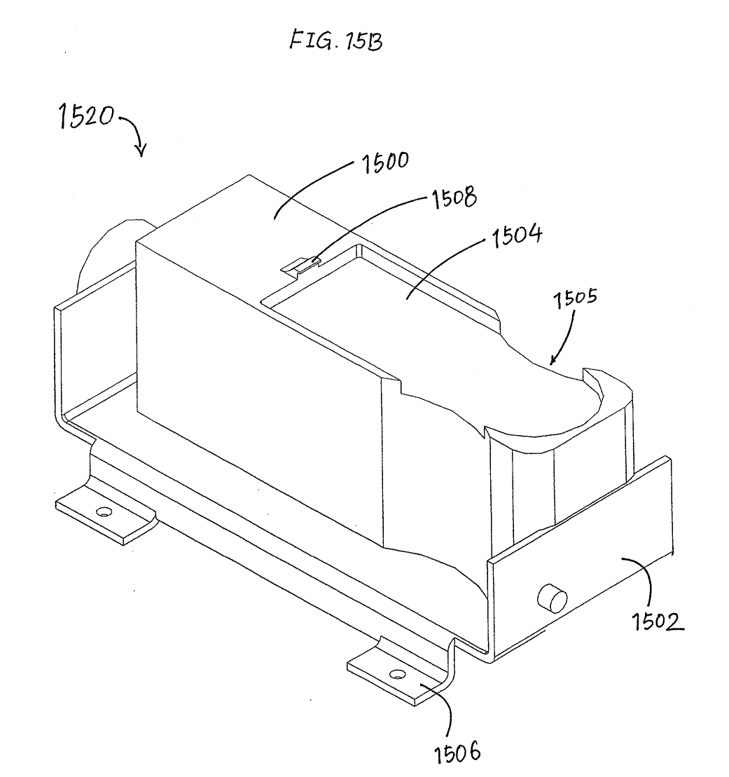

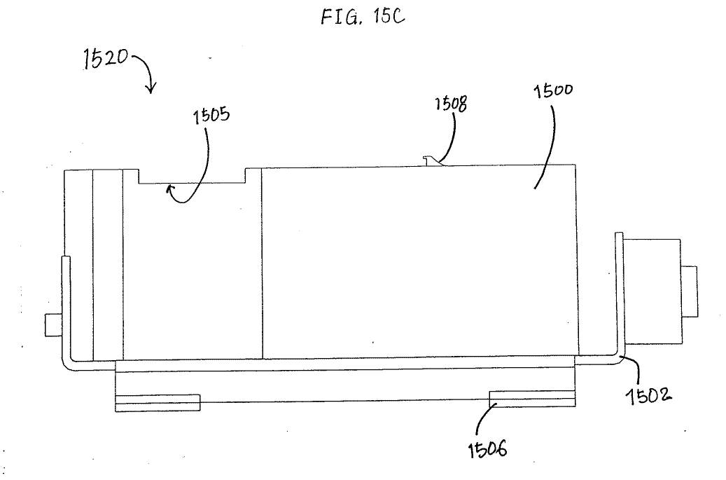

FIGS. 15A-15C show an exemplary sample holder tray assembly (1520), which may be used to contain and position a cartridge containing a test strip, such as a test strip described herein. As shown there, sample holder tray assembly (1520) comprises a sample holder (1500) and a tray (1502). Sample holder (1500), in turn, comprises a recess (1504), grooves (1505), and a cartridge retainer (1508). Recess (1504) and grooves (1505) may be sized and shaped according to the dimensions and geometry of a cartridge to be received by the sample holder, such as cartridge (111) (FIG. 2A). Grooves (1505) may, for example, enhance the ease of cartridge installation and/or removal. A variety of different methods may be used to secure a cartridge within recess (1504). For example, a cartridge may be secured by friction-fit, adhesion, and/or using a snap-fit or a retainer similar to retainer (1508). In some variations, a cartridge may be integrally formed with sample holder (1500). Sample holder (1500) may be of any appropriate size, and may comprise a plurality of grooves and/or other features configured to retain more than one cartridge.

As shown in FIGS. 15A-15C, sample holder (1500) is coupled to tray (1502). Sample holder (1500) may be permanently coupled (e.g., melded to) tray (1502), or it may be temporarily coupled to tray (1502). In certain variations, a sample holder and tray may be integral with each other. In some variations, a non-permanent coupling between a sample holder and a tray may allow for re-use of the tray, while the sample holder may be disposed of after use. Alternatively, both the tray and the sample holder may be disposed of after use. The tray may be sized and shaped to hold a variety of sample holders (e.g., a variety of sample holders (1500)) that may be configured to retain a variety of cartridges. Tray (1502) may also be configured to hold multiple sample holders (1500). As depicted in FIGS. 15A and 15B, tray (1502) may comprise attachment features (1506). Attachment features (1506) are apertures configured for the passage of a screw; however other features such as notches, clips, protrusions and the like may be used to attach tray (1502) with other components. For example, sample holder tray assembly (1520) may be attached to a motorized beam that positions the sample in sample holder (1500) for testing and analysis.

Certain variations of diagnostic systems may have one sample holder tray assembly, while other variations may have a plurality of sample holder tray assemblies. Additionally, while system (100) is shown with one optical module (101) which scans and reads out the result from a test strip, other variations of diagnostic systems may have multiple optical modules or test strip readers. In some variations, a master module may drive one or several slave modules. A master module may comprise an optical module, a motorized tray drive with multiple cartridges, an embedded PC, an electrical interface (e.g., with a slave module), and user interface (e.g., touch screen, display, and/or input device such as a mouse or keyboard). A slave module may comprise an optical module, a motorized tray drive with multiple cartridges, and an electrical interface (e.g., to a master module and/or other slave modules). A single master module may be daisy-chained to multiple slave modules, and may control the actuation of all tray drives and optical modules, which may enable the diagnostic system to analyze multiple cartridges simultaneously. Other system configurations may also be used, as described in detail below.

For example, a slave module may be used to incubate test strips prior to scanning by a master module. A master module may control the duration, temperature, light levels, and other conditions of the test strips retained in a slave module during the incubation period. At the conclusion of the incubation period, the embedded computing device of the master module may signal the ejection of the test strips from the slave module to be loaded for scanning in the master module. This may help to increase the throughput of a diagnostic system. Alternatively or additionally, test strips may be incubated in another environment, such as a tissue culture hood, clean room, etc., and subsequently manually loaded in a master module for scanning and reading. Where a slave module comprises an optical module, it may also receive scan commands from the master module after the incubation period. The scan data from the slave module may undergo preliminary processing, and then may be transmitted to the master module for storage and further analysis. Slave modules may comprise some circuitry to detect status and/or error conditions, and in some variations, may comprise acoustic speakers and/or tactile interfaces to provide feedback regarding the status of the test strips and/or the state of the optical module. In some variations, the master module may have internet or network connectivity (e.g., Ethernet connectivity), and a user may control and program the master and slave modules from a remote site.

Master modules may also have a user display, such as an LCD screen with a resolution of about 800×480 pixels and a diagonal length of about 7 inches, or a resolution of about 1024×600 pixels and a diagonal length of about 9 inches. The display or screen may be fluid-resistant. In some variations, the user display may be a touch screen, or a keyboard and/or mouse may be used to interact with the module.

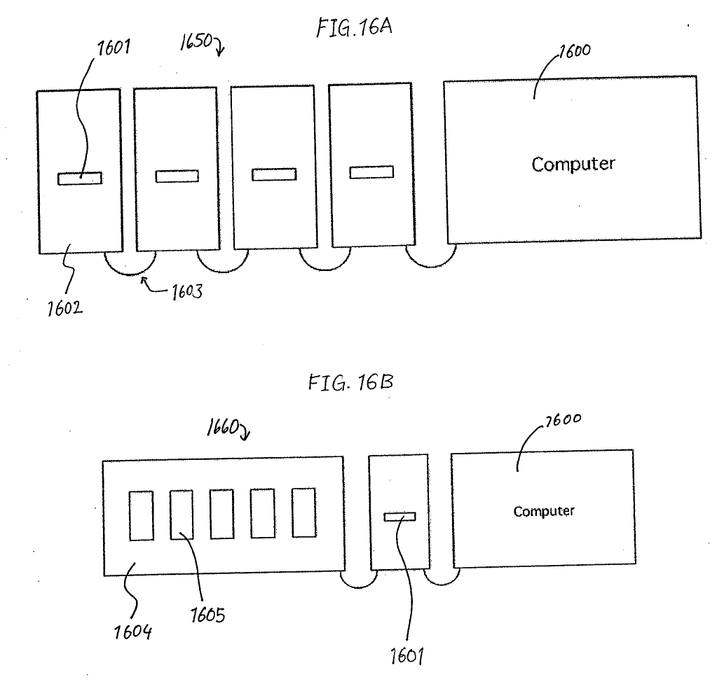

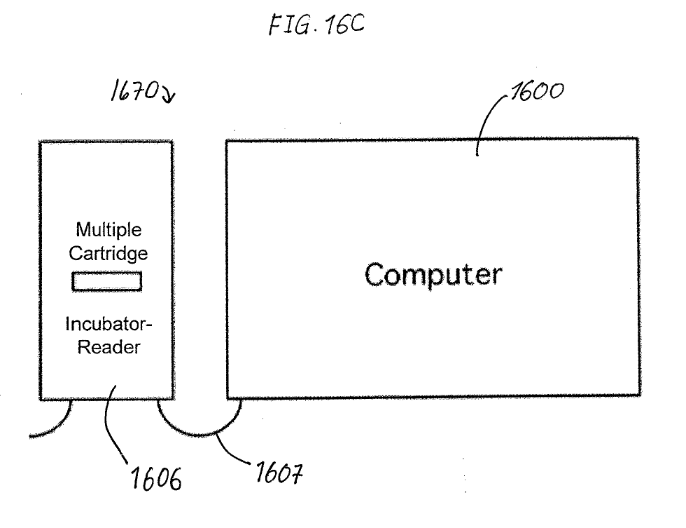

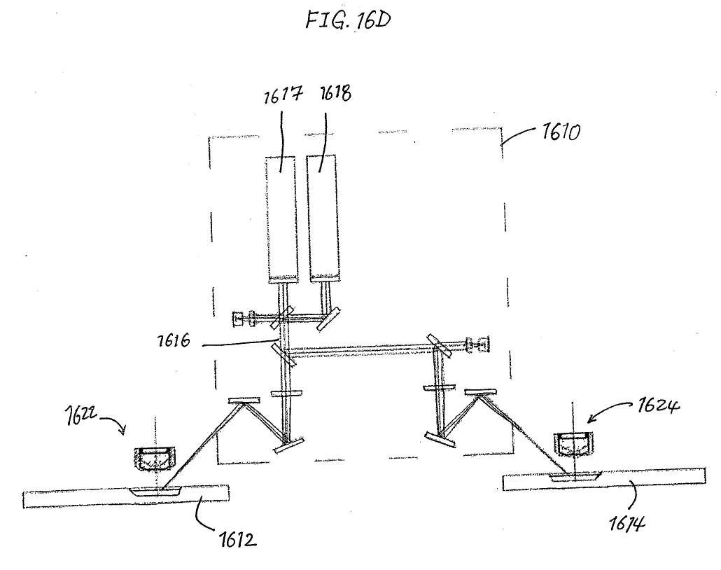

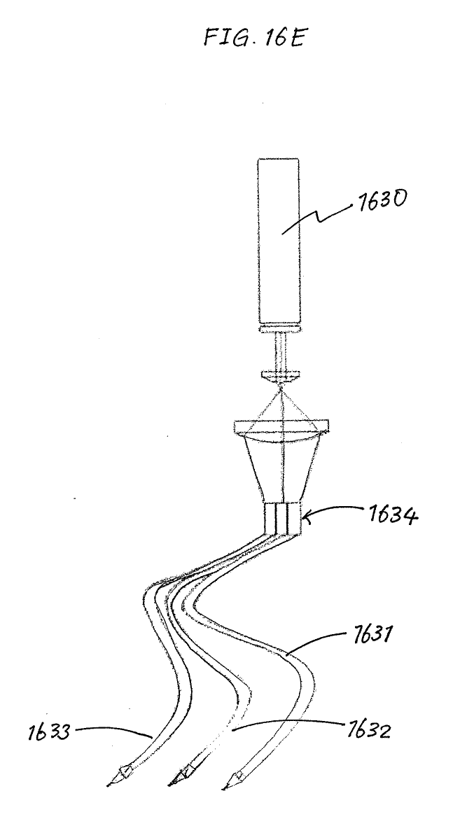

For example, FIG. 16A depicts one variation of a diagnostic system (1650) comprising a plurality of cartridges (1602) that retain test strips (not shown) and a plurality of readers (1601). In this variation, each reader is configured to read one cartridge, where the reader may read the result indicated by the test strip, and may also detect humidity levels and read barcodes that identify the test strip type. Additionally, in certain variations, readers (1601) may perform cartridge incubation. As shown in FIG. 16A, readers (1601) are connected to each other in a daisy-chain formation, via an electrical interface (1603), with the final reader connected to a controller computer (1600). This daisy-chain configuration of multiple readers (1601), with each reader configured to scan the test result of one cartridge (1602), may allow for simple scalability and high throughput, for example.

FIG. 16B shows another variation of a diagnostic system (1660). As shown there, diagnostic system (1660) comprises a single reader (1601) and an incubator (1604) with multiple cartridges (1605), where incubator (1604), reader (1601), and computer (1600) are connected in a daisy-chain configuration. Multiple cartridges (1605) loaded into incubator (1604) may be analyzed sequentially, and computer (1600) may maintain a database that determines the scan time of each cartridge. This variation may provide for relatively efficient utilization of reader (1601), and may allow for incubator scalability.

Another configuration of a diagnostic system (1670) is depicted in FIG. 16C. As shown there, multiple cartridges, an incubator, and a reader may be combined into one module (1606). The incubator may be used to expedite the binding of the analytes and analyte-binding agents, and/or to preserve the reactivity of the analytes and compounds of interest. Interface (1607) with computer (1600) may comprise multiple reader channels that allow for high throughput processing of cartridges.

In some variations in which a tray has a particular configuration, one or more other components of the system may be rearranged or varied to accommodate that configuration. As an example, FIG. 16D shows an excitation module (1610) configured to apply excitatory beams to two separate cartridges (1612) and (1614). Laser beam (1616) is a combination of beams from lasers (1617) and (1618), but is split into two beams that are ultimately directed toward separate cartridges (1612) and (1614). Each cartridge has its own detector module (1622) and (1624), which may or may not be identical to each other. Excitation module (1610) has a configuration that may allow for relatively high throughput testing and analysis of cartridges. The optics of excitation module (1610) may be arranged in any configuration suitable to match the configuration of cartridges (1612) and (1614) for effective application of excitatory beams.

In some cases, fiber-coupled lasers may be used to adequately access a tray and the cartridges positioned on the tray. For example, FIG. 16E shows a laser (1630) that applies an excitatory beam that is focused onto a fiber hub (1634). Fiber hub (1634), in turn, distributes the laser beam via optical fibers (1631), (1632), and (1633). While three optical fibers are shown, other variations may comprise a different number of optical fibers (e.g., to match the number of test cartridges). In some variations, laser (1630) may be a fiber-coupled laser diode, which may reduce the number of components in the excitation module. The use of fiber optics may accommodate a large variety of cartridge and tray configurations, and may reduce the complexity of motorized tray assemblies (e.g., may require less movement of cartridges and/or the excitation module).

As shown in FIGS. 16A-16C, some variations of diagnostic systems may be connected to an external computer (1600). However, in certain variations, a diagnostic system may comprise an embedded processor computer (PC). The embedded processor may be housed integrally within a housing of the system (e.g., housing (102) shown in FIG. 1A), or may be housed in a separate housing external to any housings of the system. Alternatively, an embedded PC may be placed in either an objective lens unit or a detector unit. The embedded PC may be custom designed, and/or proprietary, or it may be commercially available, for example, a standardized PC form factor such as PC/104, or any Windows Compatible PC that is appropriately sized for the system housing. To reduce the space occupied by the diagnostic system, the embedded PC may be relatively small (e.g., approximately 3.6 by 3.8 inches). The embedded PC may be chosen based on the demands of the software architecture that may be required to operate the diagnostic system. A variation of a software system is described in more detail below.

### Embedded Computing Device

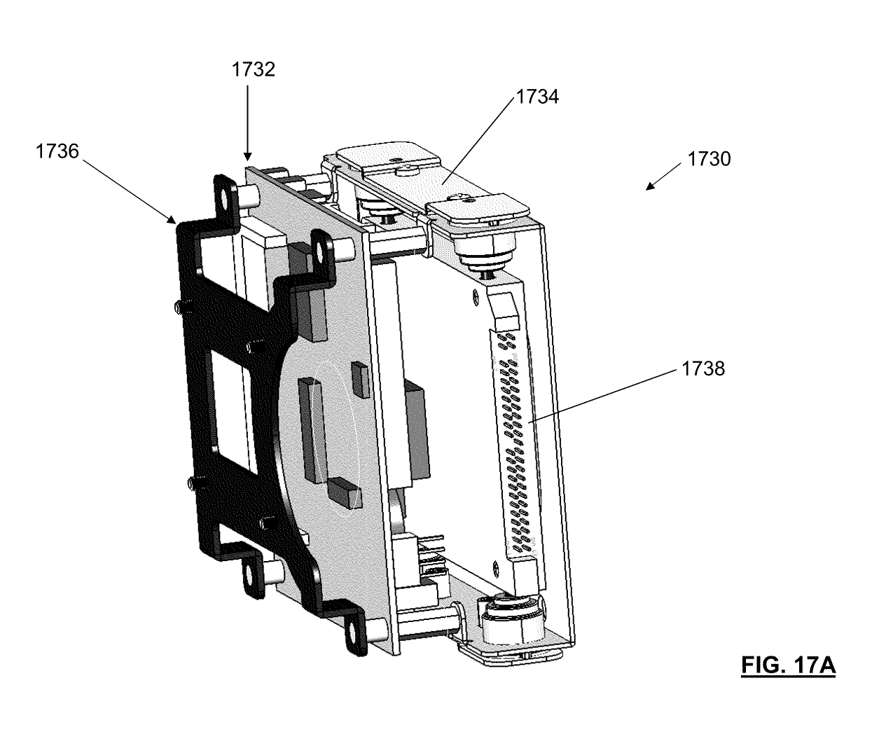

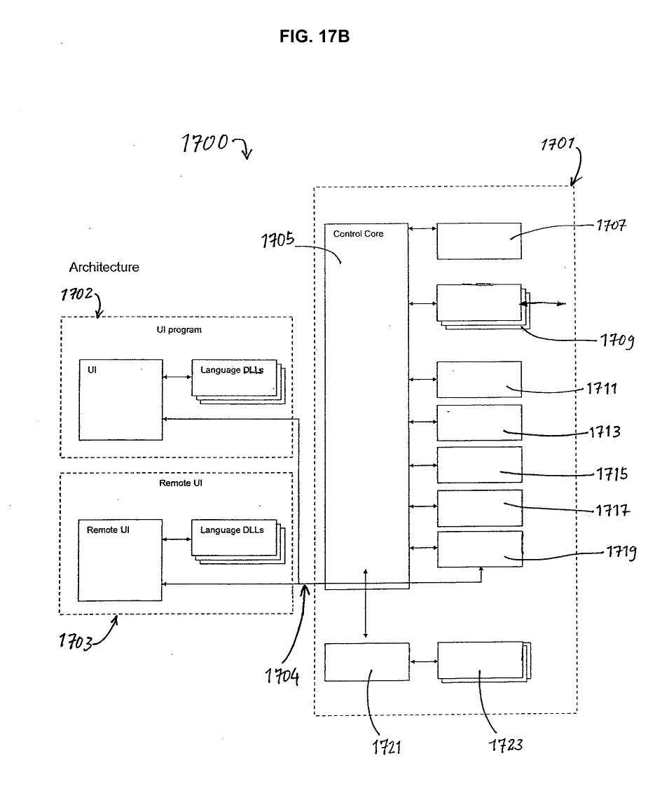

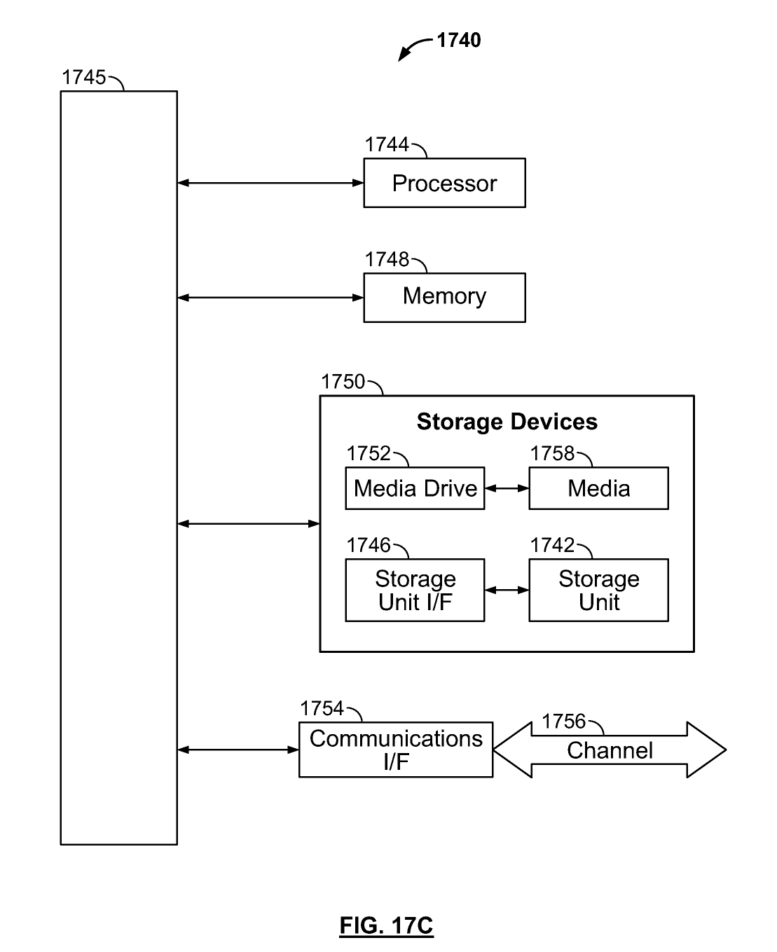

FIG. 17A depicts an example of an embedded computing device (1730) that may be used to control and calibrate a diagnostic system. As shown there, embedded computing device (1730) comprises a motherboard (1732), a hard drive (1734) that is electrically connected to the motherboard, and a mounting bracket (1736) that may be used to secure the embedded computing device to a housing of the diagnostic detection system. In some variations, hard drive (1734) may have at least about 30 gigabytes of memory. Examples of a motherboard (1732) that may be suitable for use in a diagnostic system include any system that has a PC/104 size or smaller. Embedded computing device (1730) also comprises a connector (1738) that is configured to connect with an electrical interface board of the diagnostic system. Connector (1738) may contain sufficient bandwidth for the receipt and transmission of scan and sensor data, and device commands, as well as internet or network connectivity. Connector (1738) may also be connected to a power supply to provide power to embedded computing device (1730). While FIG. 17A depicts one exemplary embedded computing device (1730), it should be understood that other embedded computing devices may also be used, as appropriate.

### External Computer

In some variations, a diagnostic system may transmit data to, and receive commands from, an external computer, such as the computer system depicted in FIG. 17C. FIG. 17C illustrates an exemplary computing system (1740) that may be employed to implement processing functionality for various aspects of the systems described here (e.g., as a user/client device, server device(s), media capture server, media data store, activity data logic/database, advertisement server, combinations thereof, and the like). Those skilled in the relevant art will also recognize how to implement the invention using other computer systems or architectures. Computing system (1740) may represent, for example, a user device such as a desktop, mobile phone, personal entertainment device, DVR, and so on, a mainframe, server, or any other type of special or general purpose computing device as may be desirable or appropriate for a given application or environment. Computing system (1740) may include one or more processors, such as a processor (1744). Processor (1744) may be implemented using a general or special purpose processing engine such as, for example, a microprocessor, microcontroller or other control logic. In this example, processor (1744) is connected to a bus (1745) or other communication medium.

Computing system (1740) may also include a main memory (1748), preferably random access memory (RAM) or other dynamic memory, for storing information and instructions to be executed by processor (1744). Main memory (1748) also may be used for storing temporary variables or other intermediate information during execution of instructions to be executed by processor (1744). Computing system (1740) may likewise include a read only memory (“ROM”) or other static storage device coupled to bus (1745) for storing static information and instructions for processor (1744).

Computing system (1740) may also include an information storage mechanism (1750), which may include, for example, a media drive (1752) and a removable storage interface (1746). Media drive (1752) may include a drive or other mechanism to support fixed or removable storage media, such as a hard disk drive, a floppy disk drive, a magnetic tape drive, an optical disk drive, a CD or DVD drive (R or RW), or other removable or fixed media drive. Storage media (1758) may include, for example, a hard disk, floppy disk, magnetic tape, optical disk, CD or DVD, or other fixed or removable medium that is read by and written to by media drive (1752). As these examples illustrate, storage media (1758) may include a computer-readable storage medium having stored therein particular computer software or data.

In alternative variations, information storage mechanism (1750) may include other similar instrumentalities for allowing computer programs or other instructions or data to be loaded into computing system (1740). Such instrumentalities may include, for example, a removable storage unit (1742) and interface (1746), such as a program cartridge and cartridge interface, a removable memory (for example, a flash memory or other removable memory module) and memory slot, and other removable storage units (1742) and interfaces (1746) that allow software and data to be transferred from removable storage unit (1742) to computing system (1740).

Computing system (1740) may also include a communications interface (1754). Communications interface (1754) may be used to allow software and data to be transferred between computing system (1740) and external devices. Examples of communications interface (1754) include a modem, a network interface (such as an Ethernet or other NIC card), a communications port (such as for example, a USB port), a PCMCIA slot and card, etc. Software and data transferred via communications interface (1754) are in the form of signals which may be electronic, electromagnetic, optical, or other signals capable of being received by communications interface (1754). These signals are provided to communications interface (1754) via a channel (1756). This channel (1756) may carry signals and may be implemented using a wireless medium, wire or cable, fiber optics, or other communications medium. Some examples of a channel include a phone line, a cellular phone link, an RF link, a network interface, a local or wide area network, and other communications channels.

### Software Architecture

FIG. 17B depicts an example of a software system (1700) that may be used to manage and control the automation and operation of a diagnostic system. Software system (1700) additionally performs data processing tasks and maintains programming interfaces so that the function of the diagnostic system may be tailored to a particular application. As shown in FIG. 17B, software system (1700) comprises a controller module (1701), a local user interface (UI) module (1702), and a remote user interface module (1703). Modules (1702) and (1703) may be implemented in hardware (e.g., a processor) that is separate from the hardware in which controller (1701) is implemented, and may all be connected by interface (1704). However, in some variations, modules (1701) and (1702) may be implemented in the same hardware assembly.

Software system (1700) may be an object-based plug-in architecture with one or more dynamic linked libraries (DLL), where each DLL may contain any number of object implementations and their associated object factories. Object factories may be loaded into an object registry upon system start-up by locating all factories in any present DLLs. Start-up configuration scripts may be provided to wire objects together into a system as desired. Examples of objects that may be included in a software system include a javascript engine (e.g., based on Mozilla SpiderMonkey/NSPR), generic property system, generic logging, IPV4 socket support, secure IPV4 socket support, web client, web server, AJAX support for web server, Relia2 interface, generic band finder, Relia2 image analyzer, generic code39 barcode decoder, Relia2-specific code 39 decoder, database engine, Relia2 database tables, Relia2 USB device interface, HTML rendering engine, generic report generator, generic UI engine, etc. Software system (1700) may also be implemented as a client-server pair where a single server runs on the instrument together with a single client. However, in other variations, additional external clients may also connect to the software system. An application program interface (API) may also be implemented, which may allow remote control through Javascript. Software system (1700) DLLs may be implemented such that the addition of one or more DLLs may not require any additional code modifications to the software system and/or to other existing DLLs.

Software system (1700) may be able to issue commands to devices in the diagnostic system according to pre-programmed or user-created routines. For example, software system (1700) may be pre-programmed to perform calibration routines, device and system diagnostics and debuggers, as well as routines to query all the sensors in the diagnostic system. Users may also use various scripting and programming languages to design customized routines suited for a desired purpose. For example, in some variations, software system (1700) may fully index patient test results, installed DLLs, connected clients and/or servers, assay tables, barcode data, etc., such that a search function may be implemented.

Data measurements from the excitation, detection, and other modules in the master and/or slave devices may be processed by software system (1700) and stored in the hard drive. Software system (1700) may process and analyze the data as described below, and may generate a report of the test results to the practitioner. The report may comprise information such as patient identification, date, test strip expiration date, lot number, test start and/or finish time, incubation time, incubation temperature, analyses performed, relevant calibration and/or standard curves, an image of the scanned strip showing the location of the fluorescent bars, relative intensity, notes from the patient and/or practitioner, interpretation of the results (e.g., positive, negative, indeterminate), etc.

Interface (1704) may be any standard electrical interface, such as a serial port interface or Ethernet, and may be a wireless interface, such as Bluetooth® or RF transmitter circuit technology. Local UI module (1702) comprises a user interface and may optionally include language capability other than English, as shown in FIG. 17B. The user interface may be graphical or command line driven. Remote UI module (1703) comprises a user interface and may optionally include language capability other than English. The information required for language capability may be stored in a database dedicated to the either of the user interface modules (1702) and (1703).

Controller module (1701) comprises a control core (1705) that manages the operation of auxiliary functional blocks, to ensure that there are no instructional hazards or invalid states. Exemplary auxiliary functional blocks may include a programming module (1707), device module (1709), curve fit module (1711), decode module (1713), database module (1715), output module (1717), web server module (1719), and assay control module (1721). Other auxiliary functional blocks may also be included (e.g., as required by the diagnostic system configurations).

Programming module (1707) manages the implementation of user-generated scripts. Programming languages that may be accommodated may include C/C++, JavaScript, MATLAB®, and the like. Depending on the programming language, programming module (1707) may also comprise a compiler. Instructions from a user-generated script may be executed by control core (1705), and may control the interaction between any auxiliary functional block. In some variations, control core (1705) may prohibit the user-generated script from accessing certain functional blocks to prevent data corruption and system malfunction.

Device module (1709) may interface with all of the individual devices of the diagnostic system to ensure that each device is properly installed, calibrated, and initialized for use. Device module (1709) may maintain a database of the identification of faulty devices or device configurations. Defective devices or erroneous device configurations may be conveyed to control core (1705), which may alert the user using output module (1717).

Curve fit module (1711) and assay control module (1721) may work in concert to analyze the data collected from a test sample. Curve fit module (1711) may implement any number of numerical models to generate a best-fit curve. Curve fit module (1711) may perform, for example, non-linear regressions, the Levenberg-Marquardt algorithm, and other smoothing functions on the collected data. The curve fit module may be a custom program, or may be a part of a statistics software package that is commercially available. In some variations, curve fit module (1711) may also perform statistical analyses to determine whether an experiment has sufficient power and precision to report a result with a minimum confidence. Statistical analyses may include analysis of variance, the student t-test, and/or confidence interval computations, as well as other parametric or non-parametric methods that are appropriate for the experiment.

Decode module (1713) may maintain a database of valid device barcodes that may be referenced by device module (1709). Invalid barcodes or a barcode of an expired or recalled component may be stored as well. Decode module (1713) may be dynamically updated from a web server through web server module (1719) for the latest barcode information. For example, the barcode may encode an internet or network address of a storage device that contains the assay table information specific to a certain assay.

Database module (1715) may be generally used by the controller to maintain system variables and data, and may be implemented using commercially available database modules, or may be implemented with proprietary code.

Output module (1717) interfaces with any output indicator, such as a display, screen, audio or visual indicator, to convey system status to the user. In some variations, output module (1717) may also manage a printer port that allows test reports and/or system reports to be printed. Output module (1717) may also present the contents of any of the system databases to the user.

Assay control module (1721) may control the actuation of all mechanical components of the diagnostic system, for example, the positioning of optical components, positioning of cartridges and trays, and any other system actuators. Assay control module (1721) may also control the output of the lasers in the excitation module, and may execute on a laser pulse sequence from programming module (1707).

Data pre-processing module (1723) may interface with the detectors (e.g., photodiodes) to collect data at a fast bus rate, store the data in data structures (such as a FIFO or LIFO buffer, multidimensional array, or other independently addressable memory), and compress the data for quick storage and transmission to control core (1705) via assay control module (1721). Data pre-processing may reduce the size of data sent to the control core by removing frequency artifacts, and/or down-sampling the data (but not below Nyquist frequency), and may increase the processing efficiency of control core (1705) and curve fit module (1711).

One or more of the modules of software system (1700), such as the data pre-processing module, may take the measured signal from a light sensor board, demodulate it if needed, and store the data in a one-dimensional array in the hard drive. In some variations, the data stored in the array is image data or an image mapping that represents the intensity of a particular light spectrum at different locations on the test strip. The data in the array may be processed to generate an estimated background. The estimated background may then be subtracted from the image mapping to determine the bands of interest and their locations on the test strip. Data encoded in the test strip barcode or RFID tag may contain information on the expected number of bands for a certain assay. The data pre-processing module may use a least-squares best match method to compare the differences between the expected number of bands against the number of bands detected in the image mapping. This may help reduce analytical errors that may arise from erroneous or noisy measurements.

The data collected by the light sensor boards may be qualitatively and/or quantitatively analyzed in several ways. One analysis may comprise computing the ratio of target analyte fluorescent intensity over the control analyte fluorescent intensity to obtain a relative intensity (RI) value. The RI value may be directly reported as a result. Another analysis may be performed by the curve fit module, and may comprise feeding the RI value into a 4-parameter or 5-parameter logistic function using curve-fit parameters provided by the assay table encoded in the test strip barcode or RFID. The resulting curve provides information such as the concentration of the target analyte (e.g., target analyte/volume in suitable units such as ng/mL). The RI value may also be compared to a cut-off constant provided by the assay table encoded in the barcode. An RI value less than or greater than the cut off constant may be reported to the practitioner as “Negative,” “Positive,” or “Indeterminate.” The RI value may also be binned according to a table of bins (which may be stored in the assay table), with an implied lower limit of zero, and with no upper limit. The result of the test may be reported by determining which part of values the input lies between, including the implied zero and infinity value. The output of the binning analysis may comprise any assay specified string associated with each limit value. For example, the bin table may be stored as an array of pairs: (limit, string), with a final value of (−, string). All value less than the largest limit are assigned the string that corresponds to the highest bin the RI value is less than. If the RI value is higher than the largest limit, the final string applies.

One analysis method that may be applied to test strips configured to detect multiple antigens using multiple bands comprises computing the RI value and the 4- or 5-parameter logistic curve as described above, and combining those results into a single result that may be used as an input to the binning analysis. For example, two bands arising from two antigens may have very different chemical “gains.” One band is effective at low doses, but saturates at intermediate doses; another is ineffective at low doses (i.e., the signal-to-noise ratio is too low) but becomes effective at higher doses where the sensitive band saturates. The results of these two bands may be combined in a variety of ways to obtain a single high dynamic range result exceeding the chemical dynamic range of any single antigen band. Each assay may encode in the barcode or RFID the data reduction method to be used in its analysis, and the results of individual analyses may be pooled to increase the dynamic range of an assay. The different analyses may be modularized, such that a new analysis method may be implemented in the computing device without modifying existing analysis methods.

Other software architecture may be included and implemented with the diagnostic systems described here. While proprietary software may be implemented, commercially available operating systems and programs may also be used.

Some variations of systems described here may be configured for connection to the Internet or to an intranet, or may have features (e.g., Bluetooth®) for cell phone connection. As an example, a system may be configured for connection to a network for health IT management. Internet or intranet connectivity may be used, for example, to transmit the original validated data to any desired location for further analysis, and/or for integration into larger data sets (e.g., for disease management and control). In certain variations, the raw data/measurements (e.g., that indicate target analyte detection) from the POC system may be analyzed locally (e.g., by the POC system itself) and/or transmitted to a remote location for interpretation and analysis. The results of the local and/or remote data analysis may be used for diagnosis and treatment decisions. The interface protocols between the local POC system and a remote analysis system may include features that ensure data security and the protection of analysis tool trade secrets. In some variations, the system may be connected to a personal health management system (e.g., iMetrikus®), which may accommodate real-time data capture from any electronic home monitoring and/or POC device. A personal health management system may store the data capture as a secure, interactive and shareable record for individuals, health professionals, payers and other healthcare companies. In certain variations, a system may be capable of being remotely monitored (e.g., via phone, via the Internet), and/or may be connected to a call center that can provide help in using the system and interpreting its results, or may be remotely controlled from a distance. As a result, the system may not require substantial on-site services. Connectivity may enhance the data management capabilities of the systems described here. Connectivity may be on a corporate, countrywide, or even worldwide basis, for example. In some variations, software and/or assay updates may be received via Internet or USB drive. Moreover, results may be stored, viewed, printed and/or downloaded via the Internet or a USB drive, for example.

For example, some variations of the systems described here may be used as part of a remote health management (RHM) and/or remote patient monitoring (RPM) system, where medical professionals may be able to control the use of the POC diagnostic system, monitor the test results, and provide medical diagnoses and advice from a remote location. In some variations, telecommunications technologies may be used to support long-distance clinical health management and assessment. For example, in an RPM system, patients may use the diagnostic device themselves to assay physiological fluid samples, and the results of the test may be reported locally to the patient, and remotely to the medical professional. The patients may, for example, assay blood samples for glucose levels, assay saliva samples for hormone levels, assay urinary samples for bacteria and/or drug by-products, etc. In some examples, non-medical personnel such as a patient's pharmacist, friend, relative, or any other non-medical professional may use the diagnostic device to assay the patient's physiological fluid samples. Patients, non-medical personnel and the like may use the systems with or without instruction by a medical professional, as appropriate. The tests may be relatively easy to use (e.g., requiring only a finger prick). In some cases, the tests may operate automatically after sample addition. Depending on the result of a diagnostic test, doctors may issue a prompt over the network to the patient to take a follow-on diagnostic test. Test results stored in the hard drive of the embedded computing device may be made available to both the patient and the medical professional as needed, and may be a part of the patient's electronic health record. An RHM and/or RPM system with a POC diagnostic device may help a medical professional determine whether a patient is complying with the recommended course of treatment and monitoring. In certain variations, tests may be automatically replenished as needed.

POC diagnostic devices with RHM and/or RPM connectivity as described above may be located in both private and public venues. Examples of private venues include a patient's residence, hospital room, bathroom, intensive care unit, automobile, clinic kiosks, athletic locker rooms, etc. Examples of public venues include airport gates and/or security checkpoints, shopping malls, pharmacies, amusement parks, retail stores, restaurants, freeway rest stops, movie theaters, gyms, athletic stadiums, hotels, etc. Other locations include the emergency room, surgery suites, and the like.

While test strips have been described above, one or more features of the test strips may be applied to other types of systems. For example, one or more of the principles described herein and characteristics or features of the devices, systems, and methods described herein may be applied to microfluidics applications. As an example, microfluidics devices may employ chambers in which a target analyte capture agent and control analyte capture agent (and/or one or more additional analyte capture agents) are co-localized (e.g., the same reaction chamber or tube). As another example, a target analyte in a fluid sample may be detected at certain locations along the channels of a microfluidics-based device. Microfluidics methods and devices are described, for example, in Martinez et al., “Three-Dimensional Microfluidic Devices Fabricated in Layered Paper and Tape,” PNAS, Vol. 105, No. 50 (Dec. 16, 2008) 19606-19611; P. K. Sorger, “Microfluidics Closes in on Point-of-Care Assays,” Nature Biotechnology, Vol. 26, No. 12 (December 2008) 1345-1378; and B. Grant, “The 3 Cent Microfluidics Chip,” The Scientist (Dec. 8, 2008), all of which are incorporated herein by reference in their entirety.

Some devices and systems may generally employ two lasers to measure two different rates in the same sample, and to thereby measure two different analytes in the same sample, regardless of whether the analytes are located on a test strip. For example, such devices, systems, and methods may be useful in some cases in which double measurements are desired (e.g., two complimentary enzyme activities).

While certain detection technologies have been described above, a diagnostic system may be configured to test and analyze samples using any of a variety of different detection technologies. For example, a diagnostic system may test and analyze samples using a flow-through technique, where a multilayer test strip comprises a reactive membrane panel that contains analyte capture constructs. A fluid sample may be applied to the multilayer test strip and may propagate to the reactive membrane panel, where the analyte of interest is captured. A subsequent step may apply an analyte detector that is tagged with a fluorophore to the test strip, which may reveal the presence and quantity of the target analyte. Another detection technique that may be used with a diagnostic system is a solid-phase technique, where a test strip (e.g., a dipstick) may comprise one or more wells that contain analyte capture constructs. A fluid sample may be applied to the well, where the analyte of interest is captured. After an incubation period, a buffer wash step may follow to reduce non-specific binding. Thereafter, an analyte detector that is tagged with a fluorophore may be applied to the well. After an incubation period, a wash step may follow, and the fluorescence measured in the well may reveal the presence and quantity of the target analyte. In either the flow-through or the solid-phase technique, the fluorescence of the analyte detector may be collected and measured by a detector module. In both techniques, a control analyte detector may be employed so that test analyte detection may be normalized with respect to control analyte detection (e.g., to remove manufacturing and environmental variability that may impact test analyte detection precision).

### Examples

The following examples are intended to be illustrative and not to be limiting.

### Example 1a

### Preparation of Test Strips and Assays

Test strips are constructed as follows.

Millipore HF 90 nitrocellulose is coated with (in order of distance from the sample application zone): control-1: 0.5 mg/ml rabbit anti-DNP mixed with cTnI test band-1: 1.2 mg/mL each of monoclonal anti-cTnI 19C7&16A11 or 0.6 mg/mL each of monoclonal anti-cTnI 19C7, TPC-6, TPC-102 & TPC-302. (Prior to coating, the antibodies are dissolved in PBS, 5% trehalose, 5% methanol for coating.) The nitrocellulose is coated using an IVEK flatbed striper at 1 μL/cm. After coating, the HF 90 nitrocellulose is incubated overnight at 37° C. and then heat-treated at 45° C. for four days.

Fluorescence conjugates of monoclonal anti-cTnI antibodies are prepared using HiLyte Fluor™ 647 fluorophore-labeled streptavidin mixed with biotin-labeled monoclonal anti-cTnI antibodies as follows.

NHS-PEO12-Biotin is used for anti-cTnI biotinylation as follows. First, 25 mM biotin stock solution is prepared by combining dimethyl sulfoxide (DMSO, Sigma) and EZ-LINK NHS-PEO12-Biotin (Pierce Biotechnology). The anti-cTnI antibodies (goat anti-cTnI antibodies (BioPacific, Cat #129C, 130C) or mouse monoclonal anti-cTnI antibodies clone 560, 625, 596 (HyTest)) are diluted with 1× PBS (ph 7.4) to a final concentration of 2.15 mg/mL, at a volume of 2.5 mL. The microliters of biotin stock solution are calculated (using 20-fold molar of biotin for antibody solution). Then, 2.5 μL biotin stock solution is added, and the result is incubated and rotated at room temperature (25° C.) for 30 minutes. A superfilter is used to remove extra free biotin using a spin column (VIVASPIN 20, 30K, Sartorius) for 5 times at 10,000 revolutions per minute for 12 minutes. The antibodies are re-suspended with 4-5 mL 1× PBS (pH 7.4), and the concentration and molar ratio of biotinylated Anti-cTnI antibody are calculated using a Pierce EZ Biotin Quantification Kit (Pierce, Cat#PI28005).

Streptavidin is conjugated with HiLyte Fluor™ 647 fluorophore as follows. First, 10 mg/mL streptavidin stock solution is prepared by combining streptavidin (AnaSpec, Cat:60659), 1× PBS buffer (pH 7.4), 10 mg/mL HiLyte Fluor™ 647 fluorophore (AnaSpec, Cat:89314), and DMSO (Sigma). The streptavidin is diluted with 1× PBS to a final concentration of 2 mg/mL, at a volume of 1.5 mL. The microliters of HiLyte Fluor™ 647 fluorophore solution are then calculated (using 15-fold molar of HiLyte Fluor™ 647 fluorophore for streptavidin solution). Next, 105 μL of HiLyte Fluor™ 647 fluorophore are added, and the result is incubated and rotated at room temperature for 2 hours. Then, superfiltration is used to remove extra free HiLyte Fluor™ 647 fluorophore using a spin column (Sartorius, VIVASPIN 20, 30K) at 4,000 revolutions per minute for 25 minutes, 15 mL each time, until the OD654 nm of the bottom solution is less than 0.08 for HiLyte Fluor™ 647 fluorophore. The conjugates are re-suspended with 3 mL 1× PBS (pH 7.4), and the concentration and molar ratio of the conjugates are calculated.

DNP-BSA is conjugated with HiLyte Fluor™ 647 fluorophore as follows. A 10 mg/mL HiLyte Fluor™ 647 fluorophore stock solution is prepared by combining DNP-BSA (made in-house), HiLyte Fluor™ 647 fluorophore (Cat: 89314, AnaSpec), and DMSO. The DNP-BSA is diluted with 1× PBS to a final concentration of 2 mg/mL, at a volume of 500 μL. The microliters of HiLyte Fluor™ 647 fluorophore solution are calculated (using 50-fold molar of HiLyte Fluor™ 647 fluorophore for DNP-BSA solution). Then, 115 μL of HiLyte Fluor™ 647 fluorophore are added, and the result is incubated and rotated at room temperature for 30 minutes. Superfiltration is used to remove extra free HiLyte Fluor™ 647 fluorophore using a spin column (NanoSep 10K, OMEGA, PALL) at 10,000 revolutions per minute for 12 minutes each time, until the OD654 nm of the bottom solution is less than 0.08. The conjugates are re-suspended with 600 μL 1× PBS (pH 7.4), and the concentration of the conjugates is calculated.

Fluorescence conjugates of DyLite-800 fluorophore labeled streptavidin and BSA-DNP are prepared by using the protocol provided in the DyLite antibody labeling kit (Pierce, Cat#PI53062).

Conjugate pads (contact bands) comprising Millipore glass fiber are prepared by mixing 0.4 mg/mL (final concentration) of biotin labeled anti-cTnI 129C &130C with 0.3 mg/mL (final concentration) of HiLyte Fluor™ 647 fluorophore labeled streptavidin conjugate. The mixture is incubated at room temperature (25° C.) for about 2-6 hours, and diluted to the proper concentration with 50% cTnI free serum. Then DyLiter-800-BSA-DNP is added to it to reach 0.1 mg/mL. Four lines are striped using a Biodot Quanti-3000 XYZ Dispensing Platform at 2.5 μL/cm. The resulting conjugate pads are dried overnight under vacuum.

Sample pads (optional separate sample application bands) are preblocked by dip coating Ahlstrom 141 pad material in: 0.6055% Tris, 0.12% EDTA.Na2, 1% BSA, 4% Tween 20 and 0.1% HBR-1. The material is dried at 37° C. for 2 hours and then vacuum dried overnight. Preblocked port 1 sample pads are cut into 10 mm wide strips using a G&L Drum Slitter.

Test cards each consisting of a 70 mm×300 mm vinyl backing, a coated 25 mm×300 mm nitrocellulose sheet, a 13 mm×300 mm conjugate pad and a 14 mm×300 mm sample pad are laminated together using a Kinematics Matrix Laminator and cut into 3.4 mm×70 mm strips. The strips are placed in cassettes described in Thayer et al., U.S. Pat. No. 6,528,323.

Assays using the strips described above are carried out in a ReLIA III Instrument (ReLIA Diagnostic Systems, Burlingame, Calif.). The cassette is placed in the cassette tray of the instrument and sample-specific information is entered. A 50 μL sample of undiluted serum or plasma or a 60 μL sample of undiluted whole blood is then added to sample port of the cassette. The addition of sample is detected by a sensor and the cassette is withdrawn into the instrument for a countdown of 20 minutes. The assay is carried out under predefined assay conditions (20 minutes at 33° C.). At the end of this time, the instrument determines the intensity of reflectance (IR) from each test and control band and the results can then be accessed using the computer interfaced with the instrument.

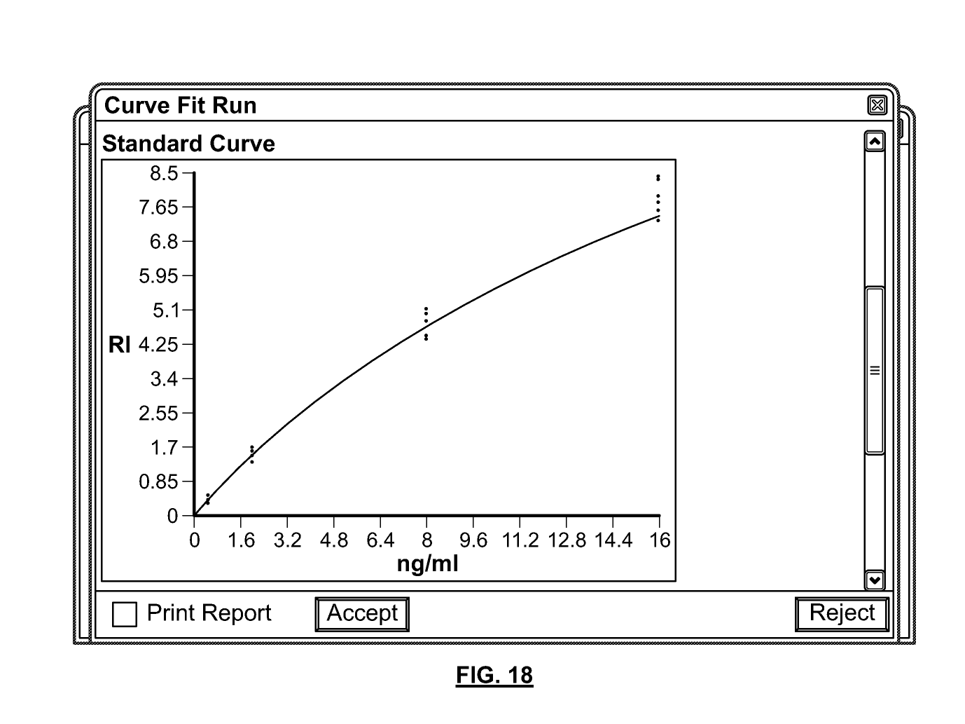

Standard samples of cTnI are prepared by diluting a concentrated solution of human cTnI into a human cTnI free serum. Results in this example are plotted as standard curves of RI (relative intensity, defined as the fluorescence intensity of the test band divided by the fluorescence intensity of control bands). Results in FIG. 18 show that the dynamic range of the RI versus cTnI concentration is between approximately 0.003 and 16 ng/mL (r>0.9977). Dynamic range is further discussed in U.S. Provisional Application Ser. No. 61/169,660, filed on Apr. 15, 2009, and in U.S. patent application Ser. No. 12/760,320, filed on Apr. 15, 2010, which are both incorporated herein by reference in their entirety.

### Example 1b

### Preparation of Alternative Test Strip Variation

While certain variations of test strips are described above, some variations of test strips may be formed by coating Millipore HF 90 nitrocellulose with a single band, separate from the sample application zone. The coating for the single band may comprise: 0.5 mg/mL rabbit anti-DNP, and either 1.2 mg/mL of each monoclonal anti-cTNI 19C7&16A11, or 0.6 mg/mL of each monoclonal anti-cTnI 19C7, TPC-6, TPC-102, and TPC-302. This coating may be immobilized on the nitrocellulose after it is deposited.

### Example 2

### cTnI Assay

cTnI labeling antibodies and a control substance were tagged with different fluorophores (HiLyte Fluor™ 647 fluorophore and DyLite-800 fluorophore), respectively, through the binding of biotin and streptavidin.

The fluorescence intensity was measured using a ReLIA III Instrument (ReLIA Diagnostic Systems, Burlingame, Calif.).

The sensitivity of cTnI was determined using a NIST cTnI reference material. Each standard cTnI was tested six times, and calculated based on the Relative Intensity (RI) of cTnI to internal control signals by using in-house developed software.

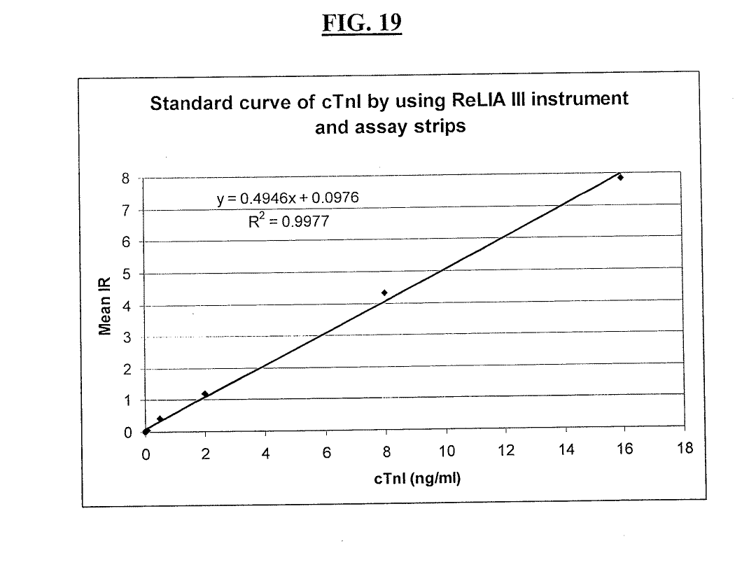

The analytical sensitivity of the cTnI assay was 0.003 ng/ml (where analytical sensitivity=mean of 0 ng/mL±3SD). The assay provided a linear response from 0.01 to 16 ng/mL, >3 logs (r>0.9977), as shown in FIG. 19 and in Table 1 below.

TABLE 1

| | RI (Test/Control) | | | | | | | | |
|---|---|---|---|---|---|---|---|---|---|
| cTnI | | | | | | | Mean | Mean | |
| (ng/mL) | strip 1 | strip 2 | strip 3 | strip 4 | strip 5 | strip 6 | (T/C) | (ng/mL) | SD |
| 0 | 0.0079780 | 0.0070114 | 0.0130242 | 0.0103327 | 0.0077754 | 0.0063628 | 0.008747 | 0.000833 | 0.001602 |
| 0.01 | 0.0198976 | 0.0213461 | 0.0260617 | 0.0171942 | 0.0214641 | 0.0177928 | 0.020626 | 0.011500 | 0.003146 |
| 0.025 | 0.0427122 | 0.0441511 | 0.0393170 | 0.0325170 | 0.0263461 | 0.0331392 | 0.036364 | 0.027667 | 0.007339 |
| 0.05 | 0.0736694 | 0.0528332 | 0.0612557 | 0.0551047 | 0.0631914 | 0.0597941 | 0.060975 | 0.054000 | 0.007975 |
| 0.5 | 0.3415454 | 0.5050479 | 0.3483841 | 0.4106406 | 0.3975278 | 0.4814537 | 0.414100 | 0.471333 | 0.083601 |
| 2 | 1.2164133 | 1.1680101 | 1.1446500 | 1.2448628 | 1.2278071 | 1.2372093 | 1.206492 | 2.084000 | 0.213768 |
| 8 | 4.0047190 | 4.1193746 | 4.3811501 | 4.1113373 | 4.8454865 | 4.4573780 | 4.319908 | 8.183500 | 0.693825 |
| 16 | 7.8978102 | 8.3182306 | 8.3761211 | 7.5512497 | 7.2809242 | 7.7415232 | 7.860976 | 17.752167 | 1.708511 |

### Example 3

### Assay Precision

Six cTnI assay strips were used to test cTnI clinical samples A and B, respectively. The concentration of cTnI from each reading was calculated based on the standard curve shown in FIG. 18. The precision for each measurement was calculating according to the equation: precision for each measurement=[(each read out−mean)/mean]*%. The precision for each measurement is shown in Tables 2 and 3 below.

TABLE 2

| Sample A | cTnI (ng/mL) | Precision |
|---|---|---|
| 1 | 0.037 | −2.6% |
| 2 | 0.035 | −7.9% |
| 3 | 0.039 | 2.6% |
| 4 | 0.041 | 7.9% |
| 5 | 0.039 | 2.6% |
| 6 | 0.039 | 2.6% |
| Mean | 0.038 | |
| SD | 0.002 | |
| CV | 5.4% | |

TABLE 3

| Sample B | cTnI (ng/mL) | Precision |
|---|---|---|
| 1 | 0.063 | 6.8% |
| 2 | 0.056 | −5.1% |
| 3 | 0.055 | −6.8% |
| 4 | 0.062 | 5.1% |
| 5 | 0.057 | −3.4% |
| 6 | 0.060 | 1.7% |
| Mean | 0.059 | |
| SD | 0.003 | |
| CV | 5.6% | |

### Example 4

### Multiplex Assays Using Fluorescence Conjugated Streptavidin

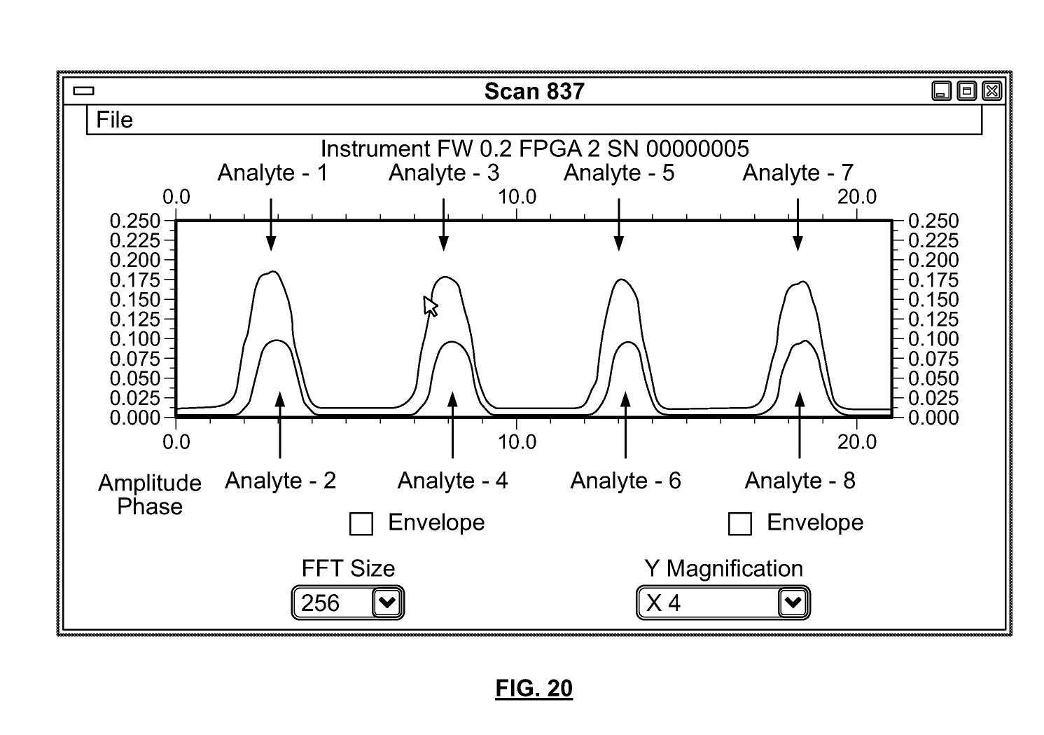

Two different fluorescence probes (HiLyte Fluor™ 647 fluorophore (0.1 mg/mL) and DyLite-800 fluorophore (0.3 mg/mL) conjugated with streptavidin were thoroughly mixed and coated on Millipore HF 90 nitrocellulose in the same location. Four different locations (each with two different colors) were coated. The strip was constructed as described above in Example 1a and was scanned with a ReLIA III Instrument (ReLIA Diagnostic Systems, Burlingame, Calif.). The fluorescence peaks of each conjugate were very well distinguished from each other. FIG. 20 shows the results of this multiplex assay.

### Example 5

### Multiplex Assays Using Fluorescence Conjugated Antibodies

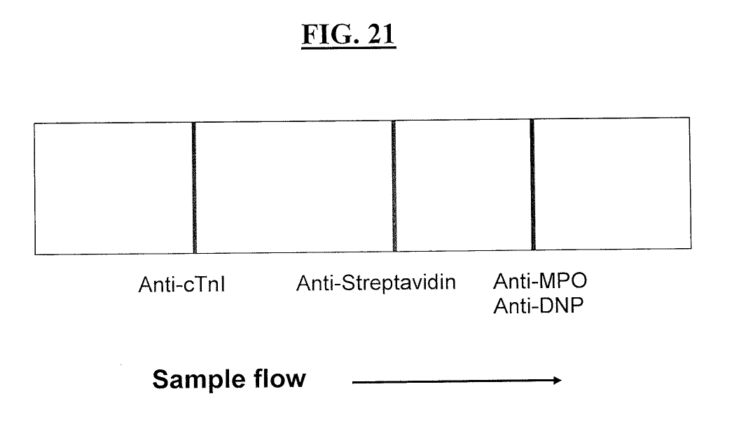

Capture antibodies of cTnI were coated on Millipore HF 90 nitrocellulose, as described in Example la above. Then, 0.0025 mg/mL of anti-streptavidin antibodies (control analyte) were coated on the nitrocellulose. A mixture of mouse anti-MPO clone 16E3 (0.25 mg/mL) and rabbit anti-DNP antibody (0.5 mg/mL, as another control analyte) was coated on the nitrocellulose at the location shown in FIG. 21.

Next, 0.4 mg/mL of HiLyte Fluor™ 647 fluorophore directly labeled anti-MPO clone 16E3 and HiLyte Fluor™ 647 fluorophore streptavidin-Biotin-cTnI antibodies (0.4 mg/mL) and 0.1 mg/mL of DyLite-800-BSA-DNP were mixed and coated on a conjugate pad (contact band).

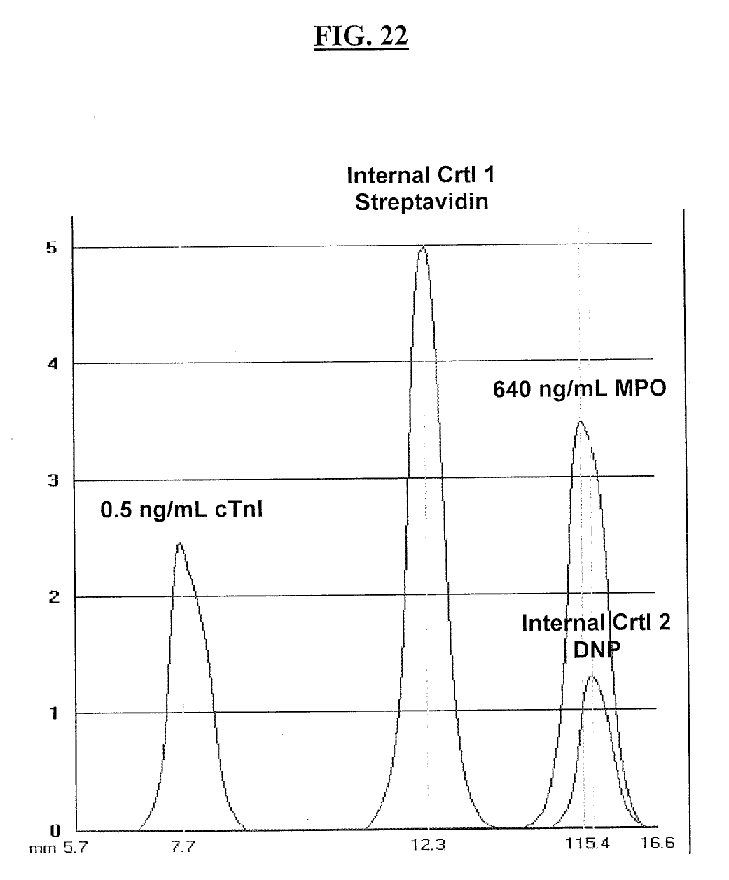

The test strip was constructed as described in Example 1a above and positioned within a cartridge. 80 uL of sample were added to a sample port in the cartridge, and the cartridge was incubated at 33° C. for 20 minutes. The test strip was then scanned with a ReLIA III Instrument (ReLIA Diagnostic Systems, Burlingame, Calif.). The results are shown in FIG. 22.

### Example 6

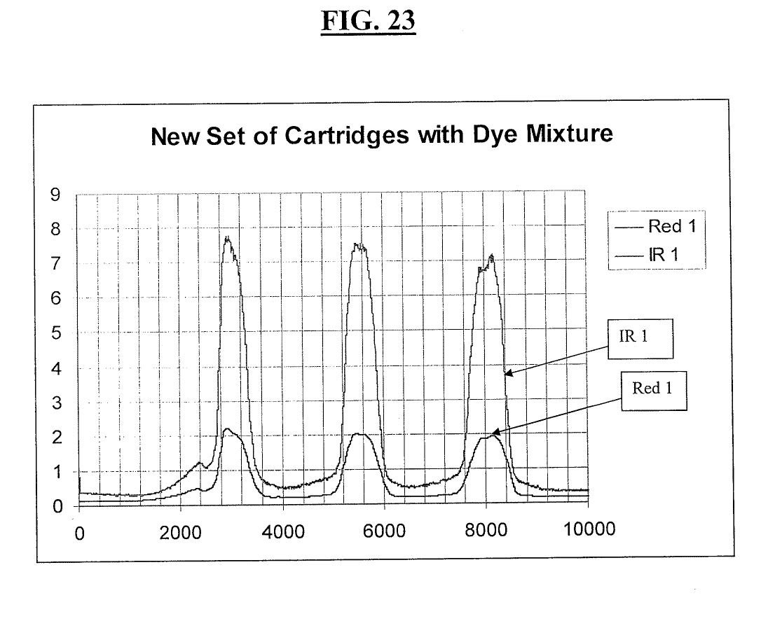

Two different fluorescence probes (HiLyte Fluor™ 647 fluorophore (0.1 mg/mL) and DyLite-800 fluorophore (0.3 mg/mL)) conjugated with streptavidin were thoroughly mixed and coated on Millipore HF 90 nitrocellulose in the same location using a Biodot Quanti-3000 XYZ Dispensing Platform at 1.0 μL/cm. Three different locations (5 mm apart) (each with two different colors) were coated. The strip was constructed as described in Example 1a above and was scanned with a ReLIA III Instrument (ReLIA Diagnostic Systems, Burlingame, Calif.). Ten strips were prepared and scanned and analyzed using a red laser, an infrared laser, and a combination of red and infrared lasers. As shown in Table 4 below, the combination of red and infrared lasers resulted in significant improvement in terms of reduction of variability (as shown by the lower coefficient of variation or CV). FIG. 23 is a graphical depiction of the results of the use of the combined red and infrared lasers.

TABLE 4

| BCG subracted | Red 1 | Red 2 | Red 3 | Red 4 | Red 5 | Red 6 | Red 7 | Red 8 | Red 9 | Red 10 | Avrage | ST Dev | CV |
|---|---|---|---|---|---|---|---|---|---|---|---|---|---|
| Peak 1 | 2.11 | 3.20 | 2.19 | 2.06 | 2.23 | 2.14 | 1.91 | 3.41 | 3.16 | 2.17 | 2.46 | 0.56 | 23% |
| Peak 2 | 1.92 | 2.89 | 1.99 | 1.83 | 2.03 | 1.82 | 1.65 | 3.10 | 2.87 | 1.87 | 2.20 | 0.54 | 24% |
| Peak 3 | 1.83 | 2.79 | 1.98 | 1.80 | 1.95 | 1.76 | 1.58 | 3.07 | 2.86 | 1.81 | 2.14 | 0.54 | 25% |
| BCG subracted | IR 1 | IR 2 | IR 3 | IR 4 | IR 5 | IR 6 | IR 7 | IR 8 | IR 9 | IR 10 | Avrage | ST Dev | CV |
| Peak 1 | 7.57 | 13.50 | 8.76 | 7.79 | 9.11 | 7.46 | 7.16 | 13.33 | 11.69 | 7.46 | 9.38 | 2.51 | 27% |
| Peak 2 | 7.32 | 12.76 | 8.41 | 7.33 | 8.63 | 6.67 | 6.54 | 12.35 | 10.74 | 6.72 | 8.75 | 2.37 | 27% |
| Peak 3 | 6.97 | 12.15 | 8.04 | 7.00 | 8.27 | 6.45 | 6.17 | 11.96 | 10.63 | 6.36 | 8.40 | 2.33 | 28% |
| BCG subracted | Ratio 1 | Ratio 2 | Ratio 3 | Ratio 4 | Ratio 5 | Ratio 6 | Ratio 7 | Ratio 8 | Ratio 9 | Ratio 10 | Avrage | ST Dev | CV |
| Peak 1 | 0.28 | 0.24 | 0.25 | 0.26 | 0.24 | 0.29 | 0.27 | 0.26 | 0.27 | 0.29 | 0.26 | 0.02 | 7% |
| Peak 2 | 0.26 | 0.23 | 0.24 | 0.25 | 0.24 | 0.27 | 0.25 | 0.25 | 0.27 | 0.28 | 0.25 | 0.02 | 7% |
| Peak 3 | 0.26 | 0.23 | 0.25 | 0.26 | 0.24 | 0.27 | 0.26 | 0.26 | 0.27 | 0.28 | 0.26 | 0.02 | 6% |

### Example 7

### Standard Curve of HA1C Assay

1.5 mg/mL mouse anti-A1C (Fitzgerald: Cat#H-12C) mixed with 0.5 mg/mL of rabbit anti-DNP (the first control) (Bethyl Laboratories) was coated on nitrocellulose (NC) (GE Healthcare) using a BioDot Quanti-3000 XYZ Dispensing platform at 1.2 uL/cm.

Donkey anti-mouse IgG (Jackson ImmunoResearch) was coated on the NC as the second control band at 0.3 mg/mL using a BioDot Quanti-3000 XYZ Dispensing platform at 1.0 uL/cm.

All antibody-coated NC was incubated at 45° C. for 4 days prior to use.

HyLite-800-labeled streptavidin was mixed with biotin-labeled Goat anti-Hemoglobin at a ratio of 1:1, and incubated at room temperature (approximately 25° C.) for 10 minutes prior to adding HyLite-647-labeled BSA-DNP. The mixture was diluted with newborn bovine serum to a concentration of 0.2 mg/mL of Hylite-800-Goat anti-Hemoglobin antibody and 0.05 mg/mL of HyLite-647-BSA-DNP. The diluted mixture was then coated on a preblocked Conjugate Pad (CP) using a BioDot Quanti-3000 XYZ Dispensing platform at 2.5 uL/cm (4 line format), and vacuum-dried overnight.

The NC, CP, absorbent pad, and sample pad were all assembled on one backing card according to the design format depicted in FIG. 3D, and cut into strips that were 3 mm in width. The strips were assembled into cassettes.

5 uL of standard HA1C whole blood tested by using an HPLC method or A1C NOW kit were added to 0.5 mL of lysing buffer. Then, 60 uL of lysed blood were added to the sample port of a strip, and incubated at room temperature (approximately 22° C.) for 5 minutes. Each strip was scanned using a ReLIA III instrument with proper laser power (e.g., about 15% laser power).

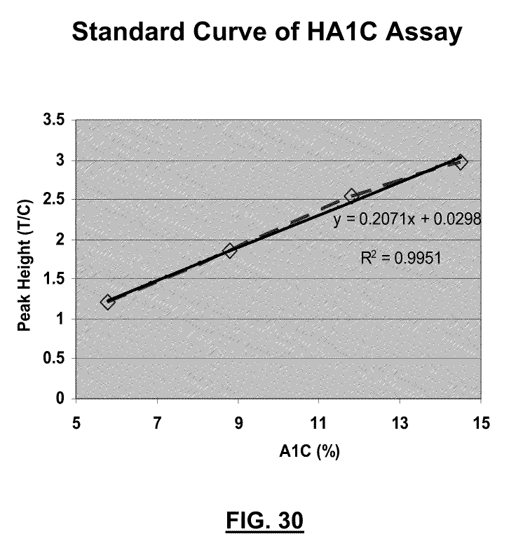

The peak heights of the test and control bands were recorded, and the ratio of the average peak height of the test band to the average peak height of the control band was calculated. This ratio was then plotted vs. % of A1C of the standard. FIG. 30 shows the resulting standard curve.

### Example 8

### Standard Curve of D-Dimer PKH (T/C)

0.5 mg/mL of mouse anti-D-Dimer clone DD3 (Hytest, Cat#8D70) mixed with 0.5 mg/mL of rabbit anti-DNP (the first control) (Bethyl Laboratories) was coated on nitrocellulose (NC) (GE Healthcare) at 1.2 uL/cm using a BioDot Quanti-3000 XYZ Dispensing platform.

Goat anti-mouse IgG (Jackson ImmunoResearch) was coated on the NC as the second control band at 0.1 mg/mL using a BioDot Quanti-3000 XYZ Dispensing platform at 1.0 uL/cm.

All antibody-coated NC was incubated at 45° C. for 4 days prior to use.

Mouse anti-D-Dimer clone DD44 was labeled with HyLite-647 (AnaSpec, Cat#89314-5) at a ratio of 1:4, and BSA-DNP was labeled with HyLite-800 (AnaSpec) at ratio of 1:1.7. The HyLite-647-labeled DD44 and HyLite-800-labeled BSA-DNP were diluted with newborn bovine serum to a concentration of 0.1 mg/mL DD44 and 0.05 mg/mL of HyLite-800-labeled BSA-DNP. They were then coated on a preblocked Conjugate Pad (CP) using a BioDot Quanti-3000 XYZ Dispensing platform at 2.5 uL/cm (4 line format), and vacuum-dried overnight.

The NC, CP, absorbent pad, and sample pad were all assembled on one backing card according to the design format depicted in FIG. 3D, and cut into strips that were 3 mm in width. The strips were assembled into cassettes.

The D-Dimer standard (Hytest Cat# 8D70) was calibrated using a Varia system and was serially diluted with newborn bovine serum from 9600 ng/mL to 150 ng/mL. Then, 60 uL of D-Dimer standard were added to the sample port of a strip, and incubated at room temperature (approximately 22° C.) for 5 minutes. Every standard concentration was tested in triplicate.

Each strip was scanned using a ReLIA III instrument with proper laser power (e.g., about 15% laser power).

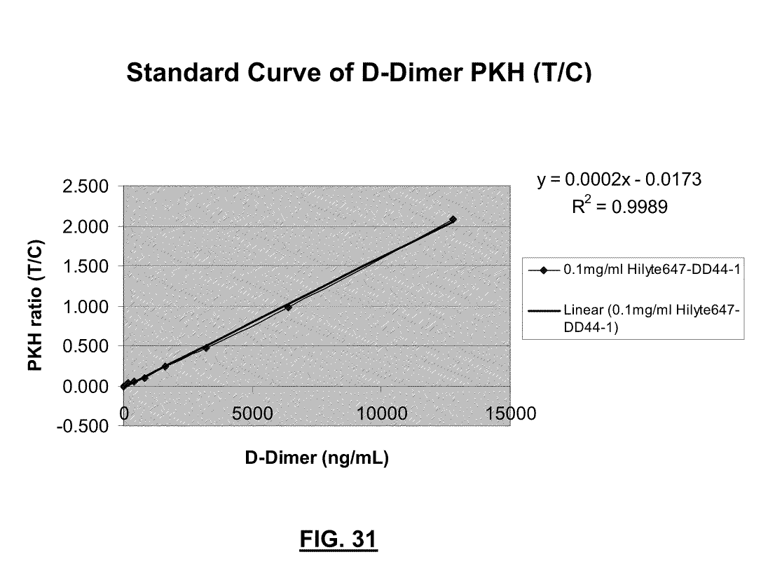

The peak heights of the test and control bands were recorded, and the ratio of the average peak height of the test band to the average peak height of the control band was calculated. This ratio was then plotted vs. ng/mL of the D-dimer standard. FIG. 31 shows the resulting standard curve.

### Example 9

### cTnl Standard Curve (PKH)

Mouse anti-cTnI (Hytest Cat#4T21, clone 19C7: 1.2 mg/mL; clone 16A11:0.8 mg/mL) mixed with rabbit anti-DNP at 0.5 mg/mL (Bethyl Laboratories) was coated as the first control (Bethyl Laboratories) on nitrocellulose (NC) (GE Healthcare) at 1.2 uL/cm using a BioDot Quanti-3000 XYZ Dispensing platform.

Rabbit anti-streptavidin (Vector) mixed with 0.5 mg/mL of BSA was coated on the NC as the second control band at 0.0025 mg/mL, using a BioDot Quanti-3000 XYZ Dispensing platform at 1.0 uL/cm.

All antibody-coated NC was incubated at 45° C. for 4 days prior to use.

HyLite-800-labeled streptavidin was mixed with biotin-labeled mouse anti-cTnI clone 625 (Hytest) and mouse anti-cTnI clone (BiosPacific, Cat#A34600) at a ratio of 1:4, and the resulting mixture was incubated at room temperature (approximately 25° C.) for 10 minutes prior to adding HyLite-647-labeled BSA-DNP. The resulting conjugate mixture was then diluted with newborn bovine serum to a concentration of 0.22 mg/mL mouse anti-cTnI antibodies and 0.05 mg/mL of HyLite-647-labeled BSA-DNP. The diluted mixture was then coated on a preblocked Conjugate Pad (CP) using a BioDot Quanti-3000 XYZ Dispensing platform at 2.5 uL/cm (4 line format), and vacuum-dried overnight.

The NC, CP, absorbent pad, and sample pad were all assembled on one backing card according to the design format depicted in FIG. 3D, and cut into strips that were 3 mm in width. The strips were assembled into cassettes.

cTnI standard (Hytest Cat#8T62) calibrated using a Beckman DXI system was serially diluted with newborn bovine serum from 100 ng/mL to 0.001 ng/mL. 80 uL of the cTnI standard were then added to the sample port of a strip, and incubated at room temperature (approximately 22° C.) for 15 minutes. Every standard concentration was tested in triplicate.

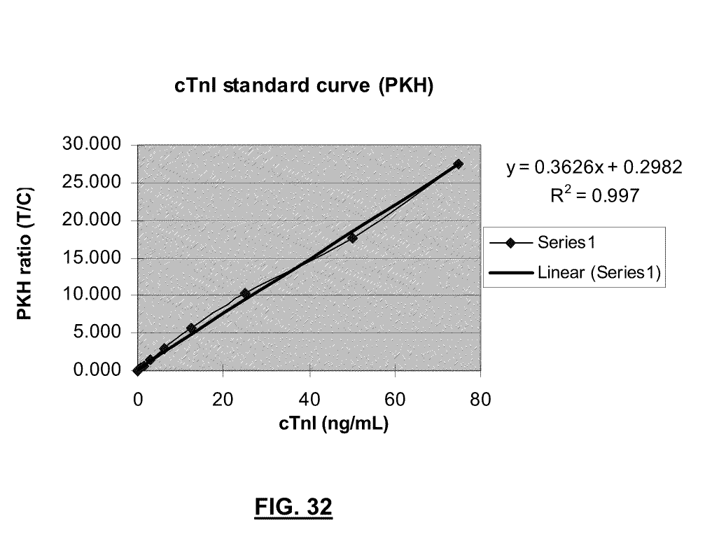

Each strip was scanned using a ReLIA III instrument with proper laser power (e.g., about 15% laser power). The peak heights of the test and control bands were recorded, and the ratio of the average peak height of the test band to the average peak height of the control band was calculated. This ratio was then plotted vs. ng/mL of the cTnI standard. FIG. 32 shows the resulting standard curve.

### Example 10

### NT-proBNP Standard Curve PKH (T/C)

Mouse anti-NT-proBNP (Hytest Cat#4NT1, clone 15F11: 1.2 mg/mL) mixed with rabbit anti-DNP at 0.5 mg/mL (Bethyl Laboratories) was coated as the first control band on nitrocellulose (NC) (GE Healthcare) at 1.2 uL/cm using a BioDot Quanti-3000 XYZ Dispensing platform.

Rabbit anti-streptavidin (Vector) mixed with 0.5 mg/mL of BSA was coated on the NC as the second control band at 0.0025 mg/mL, using a BioDot Quanti-3000 XYZ Dispensing platform at 1.0 uL/cm.

All antibody-coated NC as incubated at 45° C. for 4 days prior to use.

HyLite-800 labeled-streptavidin was mixed with biotin-labeled mouse anti-NT-proBNP (Hytest Cat#4NT1, clone 5B6:clone 11D1=2:1) at a ratio of 1:1.8, and incubated at room temperature (approximately 25° C.) for 10 minutes prior to adding HyLite-647-labeled BSA-DNP. The conjugate mixture was diluted with newborn bovine serum to a concentration of 0.22 mg/mL mouse anti-NT-proBNP antibodies and 0.05 mg/mL of HyLite-647-labeled BSA-DNP. The diluted mixture was then coated on a preblocked Conjugate Pad (CP) using a BioDot Quanti-3000 XYZ Dispensing platform at 2.5 uL/cm (4 line format), and vacuum-dried overnight.

The NC, CP, absorbent pad, and sample pad were all assembled on one backing card according to the design format depicted in FIG. 3D, and cut into strips that were 3 mm in width. The strips were assembled into cassettes.

NT-proBNP standard (Hytest Cat#8T62) calibrated using a Beckman DXI system was serially diluted with newborn bovine serum from 45,000 pg/mL to 0.499 pg/mL. Then, 60 uL of the NT-proBNP standard were added to the sample port of a strip, and incubated at room temperature (approximately 25° C.) for 5 minutes. Every standard concentration was tested in triplicate.

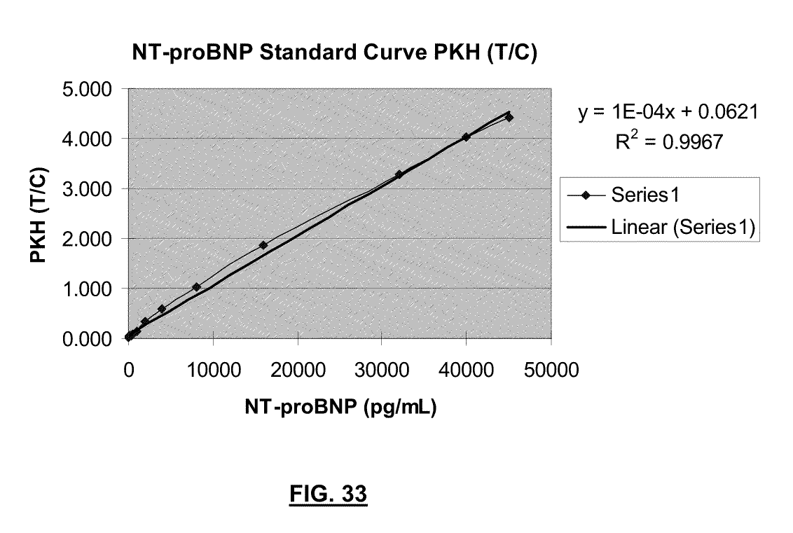

Each strip was scanned using a ReLIA III instrument with proper laser power (e.g., 15% for 0 to 500 pg/mL, 7.86% for other concentrations). The peak heights of the test and control bands are recorded, and the ratio of the average peak height of the test band to the average peak height of the control band was calculated. This ratio was then plotted vs. pg/mL of the NT-proBNP standard. FIG. 33 shows the resulting standard curve.

### Example 11

### FABP Standard Curve PKH (T/C)

Mouse anti-H-FABP (Hytest Cat#4F29), clone 9E3: 1.0 mg/mL) mixed with rabbit anti-DNP at 0.5 mg/mL (Bethyl Laboratories) was coated as the first control band on nitrocellulose (NC) (GE Healthcare) at 1.2 uL/cm.

Rabbit anti-streptavidin (Vector) at 0.0025 mg/mL mixed with 0.5 mg/mL of BSA was coated on the NC as the second control band, using a BioDot Quanti-3000 XYZ Dispensing platform at 1.0 uL/cm.

All antibody-coated NC was incubated at 45° C. for 4 days prior to use.

HyLite-800 labeled-streptavidin is mixed with biotin-labeled mouse anti-H-FABP (Hytest Cat#4F29, clone 10E1) at a ratio of 1:1.8, and incubated at room temperature (approximately 25° C.) for 10 minutes prior to adding HyLite-647-labeled BSA-DNP. The conjugate mixture was diluted with newborn bovine serum to a concentration of 0.22 mg/mL mouse anti-H-FABP antibodies and 0.05 mg/mL HyLite-647-labeled BSA-DNP. The diluted mixture was then coated on a preblocked Conjugate Pad (CP) using a BioDot Quanti-3000 XYZ Dispensing platform at 2.5 uL/cm (4 line format), and vacuum-dried overnight.

The NC, CP, absorbent pad, and sample pad were all assembled on one backing card according to the design format depicted in FIG. 3D, and cut into strips that were 3 mm in width. The strips were assembled into cassettes.

H-FABP standard (Hytest Cat#8F65) was serially diluted with newborn bovine serum from 200 ng/mL to 0.31 ng/mL. Then, 60 uL of the H-FABP standard were added to the sample port of a strip, and the strip was incubated at room temperature (approximately 25° C.) for 5 minutes. Every standard concentration was tested in triplicate.

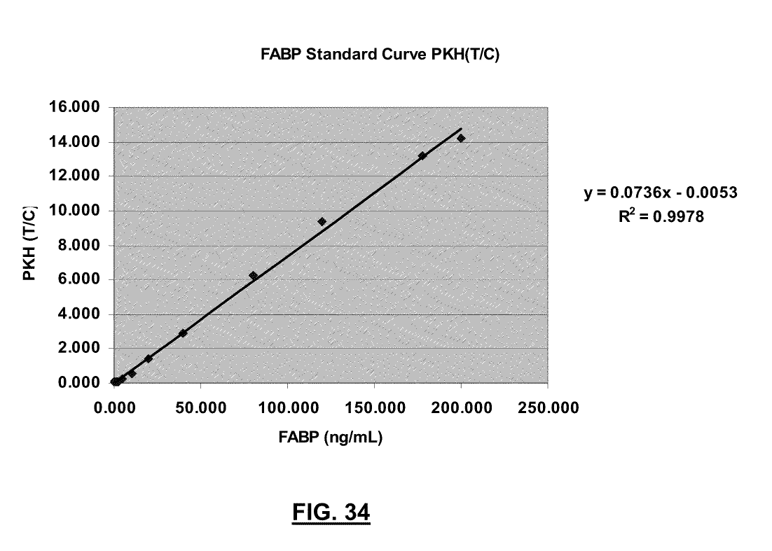

Each strip was scanned using a ReLIA III instrument with proper laser power (e.g., 15% for 0 to 40 pg/mL, 3.25% for other concentrations). The peak heights of the test and control bands were recorded, and the ratio of average peak height of the test band to the average peak height of the control band was calculated. This ratio was then plotted vs. ng/mL of the H-FABP standard. FIG. 34 shows the resulting standard curve.

### Example 12

### MPO Standard Curve PKH(T/C)

Mouse anti-MPO (Hytest Cat#4M43), clone 16E3: 0.5 mg/mL) mixed with rabbit anti-DNP at 0.5 mg/mL (Bethyl Laboratories) was coated on nitrocellulose (NC) (GE Healthcare) as the first control band at 1.2 uL/cm using a BioDot Quanti-3000 XYZ Dispensing platform.

Rabbit anti-streptavidin (Vector) at 0.0025 mg/mL mixed with 0.5 mg/mL of BSA was coated as the second control band using a BioDot Quanti-3000 XYZ Dispensing platform at 1.0 uL/cm.

All antibody-coated NC was incubated at 45° C. for 4 days prior to use.

HyLite-800-labeled streptavidin was mixed with biotin-labeled mouse anti-MPO (Hytest Cat#4M43, clone 16E3) at a ratio of 1:1.8, and incubated at room temperature (approximately 25° C.) for 10 minutes prior to adding HyLite-647-labeled BSA-DNP. The conjugate mixture was diluted with newborn bovine serum to a concentration of 0.22 mg/mL mouse anti-MPO antibodies and 0.05 mg/mL HyLite-647-labeled BSA-DNP. The diluted mixture was then coated on a preblocked Conjugate Pad (CP) using a BioDot Quanti-3000 XYZ Dispensing platform at 2.5 uL/cm (4 line format), and vacuum-dried overnight.

The NC, CP, absorbent pad, and sample pad were all assembled on one backing card according to the design format depicted in FIG. 3D, and cut into strips that were 3 mm in width. The strips were assembled into cassettes.

MPO standard (Hytest Cat#8M80) was serially diluted with newborn bovine serum from 2000 ng/mL to 10 ng/mL. Then, 60 uL of MPO standard were added to the sample port of a strip, and the strip was incubated at room temperature (approximately 25° C.) for 5 minutes. Every standard concentration is tested was triplicate.

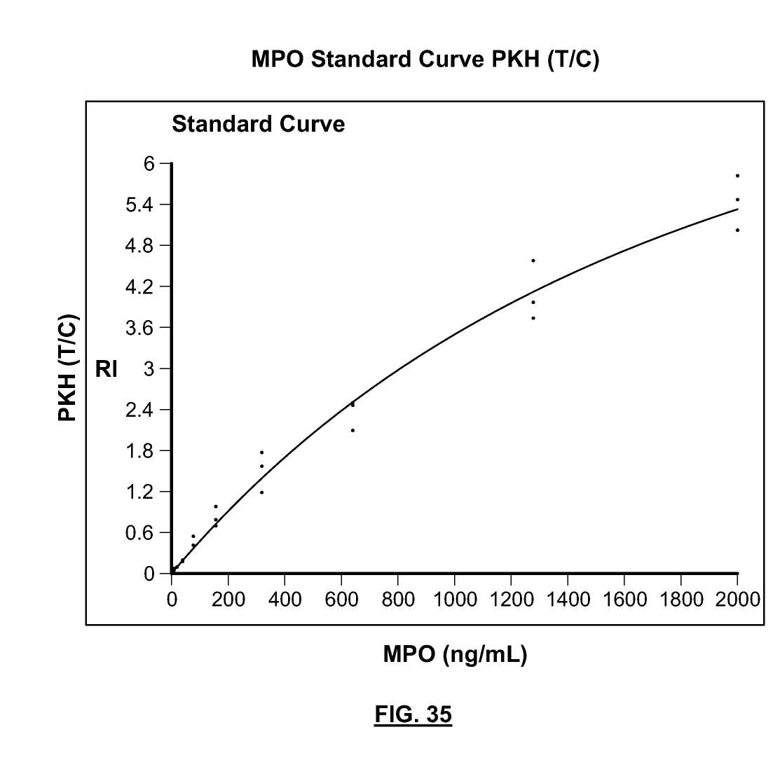

Each strip was scanned using a ReLIA III instrument with proper laser power (e.g., from about 0.78% to 100%, depending on the intensity of fluorescent signal that is measured). The peak heights of the test and control bands were recorded, and the ratio of the average peak height of the test band to the average peak height of the control band was calculated. This ratio was then plotted vs. ng/mL of the MPO standard. FIG. 35 shows the resulting standard curve.

### Example 13

### Relia III Assay Performance

Experiments were performed as described in Examples 7-12 above. The results are summarized in Table 5.

As used herein, the analytical sensitivity of an assay is indicative of that assay's ability to detect a low concentration of a given substance in a biological sample. Analytical sensitivity may be determined in one of two ways: 1) Empirically, by testing serial dilutions of specimens with a known concentration of the target substance; or 2) Statistically, by testing multiple negative specimens (0 ng/mL) and using 2 or 3 standard deviations (SD) above the mean as the lower limit of detection (Analytical Sensitivity). The Statistical Method is used to determine the analytical sensitivity (2SD) for each assay. The results are shown in Table 5 below. As shown in Table 5, the tested assays exhibited very good analytical sensitivity. Additionally, the clinical cutoff is shown in Table 5, and is a metric that may be used to indicate whether the sample may appropriately be used to characterize the test strips.

TABLE 5

| | | | Analytical | | Correlation | Dynamic |
|---|---|---|---|---|---|---|
| | Clinical Cutoff | | Sensitivity | CV (%) | (r) | Range |
| cTnI* | 0.15 | ng/ml | 0.003 ng/ml | 5.4% @ 0.04 ng/ml | 0.9988 | ~3 logs |
| | | | | | | (0.01~16) |
| H-FABP** | 10 | ng/ml | 0.04 ng/ml | 6.51% @ 6.8 ng/ml | 0.9915 | ~3 logs |
| | | | | | | (0.25~166.91) |
| MPO** | 160 | ng/ml | 0.31 ng/ml | 5.76% @ 106.6 ng/ml | 0.9916 | ~3 logs |
| | | | | | | (0.81~2,927) |
| NT-proBNP** | I: | 125~450 | 0.00019 ng/ml | 6.70% @ 0.105 ng/ml | 0.9938 | ~5 logs |
| | II: | 450~1,700 | | | | (0.55~44,620) |
| | III: | 1,700~4,200 | | | | |
| | IV: | ≧4,200 ng/ml | | | | |
| HbA1C** | | | 0.92% | | 0.9958 | 2.91~18.47 |

While the devices, systems, and methods have been described in some detail here by way of illustration and example, such illustration and example is for purposes of clarity of understanding only. It will be readily apparent to those of ordinary skill in the art in light of the teachings herein that certain changes and modifications may be made thereto without departing from the spirit and scope of the appended claims. Additionally, assays and related devices, systems and methods are also described, for example, in U.S. Pat. Nos. 6,767,710; 7,229,839; 7,297,529; 7,309,611; and 7,521,196, each of which is incorporated herein by reference in its entirety.

What is claimed is:

1. A test strip configured to receive a sample for detection of an analyte therein, the test strip comprising:
a substrate; and
a coating on a portion of the substrate, the coating comprising a combination of a first analyte capture agent configured to bind to a first analyte and a second analyte capture agent configured to bind to a second analyte that is different from the first analyte.

2. The test strip of claim 1, wherein the coating comprises a mixture of the first and second analyte capture agents.

3. The test strip of claim 1, wherein the second analyte is a control analyte.

4. The test strip of claim 1, further comprising an analyte binding agent and a control analyte that are each labeled with detectable markers.

5. The test strip of claim 4, wherein the analyte binding agent is labeled with a first fluorophore.

6. The test strip of claim 5, wherein the control analyte is labeled with a second fluorophore that is different from the first fluorophore.

7. The test strip of claim 1, wherein the substrate comprises nitrocellulose.

8. The test strip of claim 1, wherein the coating forms a first band on the substrate.

9. The test strip of claim 8, further comprising a second band configured for addition of the sample thereto.

10. The test strip of claim 9, wherein the first band is from about 3 millimeters to about 5 millimeters from the second band.

11. The test strip of claim 1, wherein the first analyte capture agent is selected from the group consisting of antibodies, engineered proteins, peptides, haptens, lysates containing heterogeneous mixtures of antigens having analyte binding sites, ligands, and receptors.

12. The test strip of claim 11, wherein the second analyte capture agent is selected from the group consisting of antibodies, engineered proteins, peptides, haptens, lysates containing heterogeneous mixtures of antigens having analyte binding sites, ligands, and receptors.

13. A method for detecting at least one analyte in a sample comprising:
applying the sample to a portion of a test strip comprising a coating comprising a first analyte capture agent configured to bind to a first analyte and a second analyte capture agent configured to bind to a second analyte that is different from the first analyte; and
applying light to the test strip,
wherein the application of light to the test strip provides an indication of whether the first analyte is present in the sample.

14. The method of claim 13, wherein the second analyte is a control analyte.

15. The method of claim 13, further comprising measuring the concentration of the first analyte in the sample.

16. The method of claim 15, wherein applying light to the test strip comprises applying light from first and second light sources to the test strip.

17. The method of claim 16, wherein at least one of the first and second light sources comprises a laser.

18. The method of claim 17, wherein the first light source comprises a first laser and the second light source comprises a second laser that is different from the first laser.

19. The method of claim 16, wherein the test strip further comprises an analyte binding agent labeled with a first fluorophore that fluoresces upon exposure to light from the first light source.

20. The method of claim 19, wherein the test strip further comprises a control analyte labeled with a second fluorophore that fluoresces upon exposure to light from the second light source.

21. The method of claim 20, wherein measuring the concentration of the first analyte in the sample comprises comparing the intensity of the fluorescence of the first fluorophore to the intensity of the fluorescence of the second fluorophore.

22. The method of claim 15, wherein the second analyte is a control analyte, and measuring the concentration of the first analyte in the sample comprises using a processor, memory resources, and software to evaluate the amount of the first analyte capture agent that is bound to the first analyte relative to the amount of the second analyte capture agent that is bound to the second analyte.

23. The method of claim 22, wherein the processor, memory resources, and software analyze the test strip at least about one second after the sample has been applied to the portion of the test strip.

24. The method of claim 13, wherein the sample comprises blood, and wherein the method further comprises passing the sample through a filter before applying the sample to the portion of the test strip.

25. The method of claim 13, wherein the first analyte capture agent is selected from the group consisting of antibodies, engineered proteins, peptides, haptens, lysates containing heterogeneous mixtures of antigens having analyte binding sites, ligands, and receptors.

26. The method of claim 25, wherein the second analyte capture agent is selected from the group consisting of antibodies, engineered proteins, peptides, haptens, lysates containing heterogeneous mixtures of antigens having analyte binding sites, ligands, and receptors.

27. A method of making a test strip configured to receive a sample for detection of an analyte therein, the method comprising:
combining a first analyte capture agent with a second analyte capture agent to form a coating material, wherein the first analyte capture agent is configured to bind to a first analyte and the second analyte capture agent is configured to bind to a second analyte that is different from the first analyte; and
applying the coating material to a portion of a substrate to form a coating on the substrate.

28. The method of claim 27, wherein the second analyte is a control analyte.

29. A point-of-care system for detecting an analyte in a sample, the point-of-care system comprising:
an apparatus comprising a first laser, a second laser that is different from the first laser, and a housing comprising a receptacle; and
a test strip configured to fit within the receptacle,
wherein the first laser is configured to apply a first beam to a location on the test strip when the test strip is positioned in the receptacle, and the second laser is configured to apply a second beam to the same location on the test strip when the test strip is positioned in the receptacle.

30. The system of claim 29, wherein the apparatus further comprises at least one mirror configured to direct application of at least one of the first and second beams to the test strip.

31. The system of claim 29, wherein the apparatus further comprises an objective lens configured to receive light emitted from the test strip.

32. The system of claim 31, wherein the apparatus further comprises a first detector configured to detect light emitted from the test strip and received through the objective lens.

33. The system of claim 29, wherein the test strip comprises a substrate and a coating on a portion of the substrate, the coating comprising a first analyte capture agent configured to bind to a first analyte and a second analyte capture agent configured to bind to a second analyte that is different from the first analyte.

34. The system of claim 33, wherein the test strip further comprises an analyte binding agent and a control analyte, and wherein the analyte binding agent and the control analyte are labeled with detectable markers.

35. The system of claim 34, wherein the analyte binding agent is labeled with a first fluorophore and the control analyte is labeled with a second fluorophore.

36. The system of claim 35, wherein the first laser emits light at a wavelength within the excitation spectrum of the first fluorophore.

37. The system of claim 36, wherein the second laser emits light at a wavelength within the excitation spectrum of the second fluorophore.

38. The system of claim 35, wherein the apparatus further comprises an objective lens configured to receive light emitted from the location of the receptacle.

39. The system of claim 38, wherein the apparatus further comprises a first detector configured to detect light emitted from the location of the receptacle and received through the objective lens.

40. The system of claim 39, wherein the first detector is configured to detect fluorescence from the first fluorophore.

41. The system of claim 40, wherein the apparatus further comprises a second detector configured to detect fluorescence from the second fluorophore.

42. The system of claim 41, wherein the apparatus further comprises a filter configured to separate fluorescence from the first fluorophore from fluorescence from the second fluorophore.

43. The system of claim 42, wherein the filter comprises a dichroic filter.

44. The system of claim 29, wherein the first laser emits light at a wavelength of about 300 nm to about 800 nm.

45. The system of claim 44, wherein the second laser emits light at a wavelength of about 300 nm to about 800 nm.

46. The system of claim 45, wherein the first laser emits light at a different wavelength from the second laser.

47. The system of claim 29, wherein the first laser comprises a laser emitting in the red region of spectrum.

48. The system of claim 47, wherein the second laser comprises an infrared laser.

49. The system of claim 29, wherein the second laser comprises an infrared laser.

50. The system of claim 29, wherein at least one of the first and second lasers is a fiber-coupled laser.

51. The system of claim 29, wherein the apparatus further comprises a photodiode.

52. The system of claim 29, wherein the apparatus is configured to measure the concentration of the first analyte to an analytical sensitivity of about 3 pg/mL.

53. The system of claim 29, wherein the apparatus is configured to measure the concentration of the first analyte to an analytical sensitivity of at least 3 pg/mL with a coefficient of variation of less than 5%.

54. The system of claim 29, wherein the system is configured to detect a plurality of analytes in a sample.

55. The system of claim 54, wherein the system is configured to detect from ten to twenty analytes on the test strip.

56. A method for detecting at least one analyte in a sample comprising:
applying the sample to a test strip;
applying a first beam from a first laser of a point-of-care diagnostic system to a location on the test strip; and
applying a second beam from a second laser of the point-of-care diagnostic system to the same location on the test strip,
wherein the application of the first and second beams to the location on the test strip provides an indication of whether the at least one analyte is present in the sample.

57. The method of claim 56, wherein the first and second beams are applied to the test strip simultaneously.

58. A method comprising:
adding a sample obtained from a subject to a point-of-care diagnostic system configured to obtain data from the sample regarding the presence or absence of one or more analytes therein, and to transmit the data in real time to a remote location where the data may be evaluated and/or incorporated into a medical record of the subject.

59. The method of claim 58, wherein the remote location is at least about 20 feet from the point-of-care diagnostic system

60. The method of claim 58, wherein the subject adds the sample to the point-of-care diagnostic system.

61. The method of claim 60, wherein the sample is added to the point-of-care diagnostic system in a non-clinical setting.

62. The method of claim 58, wherein the point-of-care diagnostic system is configured for operation by an operator without medical training.

63. The method claim 58, wherein the point-of-care diagnostic system is configured to transmit the data to the remote location telephonically.

64. The method of claim 58, wherein the point-of-care diagnostic system is configured to transmit the data to the remote location via the Internet.

65. The method of claim 58, wherein the point-of-care diagnostic system is configured to transmit the data to the remote location via an intranet.

66. The method of claim 58, wherein the point-of-care diagnostic system comprises a test strip, and wherein adding the sample to the point-of-care diagnostic system comprises applying the sample to the test strip.

67. The method of claim 66, wherein the test strip comprises a substrate and a coating on a portion of the substrate, the coating comprising a combination of a first analyte capture agent configured to bind to a first analyte and a second analyte capture agent configured to bind to a second analyte that is different from the first analyte.

68. The method of claim 67, wherein the data includes the concentration of at least one of the first and second analytes.

69. The method of claim 58, wherein the point-of-care diagnostic system comprises an apparatus comprising a first laser, a second laser, and a housing comprising a receptacle, and a test strip configured to fit within the receptacle.

70. The method of claim 69, wherein adding the sample to the point-of-care diagnostic system comprises applying the sample to the test strip when the test strip is positioned in the receptacle.

71. The method of claim 70, further comprising applying a first beam from the first laser to the test strip, and applying a second beam from the second laser the test strip.

72. A method comprising:
adding a sample obtained from a subject to a point-of-care diagnostic system,
wherein the point-of-care diagnostic system is configured for operation by an operator in a remote location.

73. The method of claim 72, wherein the remote location is at least about 20 feet from the point-of-care diagnostic system

74. The method of claim 72, wherein the point-of-care diagnostic system is configured to transmit data obtained from the sample to the remote location in real time.

75. The method of claim 72, wherein the subject adds the sample to the point-of-care diagnostic system.

76. The method of claim 75, wherein the sample is added to the point-of-care diagnostic system in a non-clinical setting.

77. The method of claim 72, wherein the point-of-care diagnostic system is configured for telephonic operation.

78. The method of claim 72, wherein the point-of-care diagnostic system is configured for operation via the Internet.

79. The method of claim 72, wherein the point-of-care diagnostic system is configured for operation via an intranet.

80. The method of claim 72, wherein the operator is a medical professional.

81. The method of claim 72, wherein the point-of-care diagnostic system is configured to be automatically refilled or replenished.

82. The method of claim 72, wherein the point-of-care diagnostic system comprises a test strip, and wherein adding the sample to the point-of-care diagnostic system comprises applying the sample to the test strip.

83. The method of claim 82, wherein the test strip comprises a substrate and a coating on a portion of the substrate, the coating comprising a combination of a first analyte capture agent configured to bind to a first analyte and a second analyte capture agent configured to bind to a second analyte that is different from the first analyte.

84. The method of claim 83, wherein the data includes the concentration of at least one of the first and second analytes.

85. The method of claim 72, wherein the point-of-care diagnostic system comprises an apparatus comprising a first laser, a second laser, and a housing comprising a receptacle, and a test strip configured to fit within the receptacle.

86. The method of claim 85, wherein adding the sample to the point-of-care diagnostic system comprises applying the sample to the test strip when the test strip is positioned in the receptacle.

87. The method of claim 86, further comprising applying a first beam from the first laser to the test strip, and applying a second beam from the second laser the test strip.
